(19) United States
(12) Patent Application Publication
(10) Pub. No.: US 2026/0325426 A1
(43) Pub. Date: Oct. 22, 2026

(54) METHODS AND COMPOSITIONS FOR PROVIDING CUSTOMIZED SKIN REGIMENS

(71) Applicant: Vibrant Holdings, LLC, Santa Clara, CA (US)

(72) Inventors: Karenah Rajasekaran, Hillsborough, CA (US); Hari Krishnan Krishnamurthy, San Jose, CA (US); Vasanth Jayaraman, Sunnyvale, CA (US); Kang Bei, San Mateo, CA (US); Tianhao Wang, Fremont, CA (US); John J. Rajasekaran, Hillsborough, CA (US); Deborah Jeyasekar, Sunnyvale, CA (US); Hannah Rajasekaran, Hillsborough, CA (US); Karthik Krishna, San Ramon, CA (US)

(21) Appl. No.: 19/662,571

(22) Filed: Apr. 29, 2026

Related U.S. Application Data
(63) Continuation of application No. PCT/US2024/053891, filed on Oct. 31, 2024.
(60) Provisional application No. 63/546,671, filed on Oct. 31, 2023.

(51) Int. Cl. A61K 8/64; A61K 8/34; A61K 8/36; A61K 8/365; A61K 8/67; A61K 8/73; A61Q 19/08; G06Q 30/0601

(52) U.S. Cl. CPC A61K 8/64; A61K 8/345; A61K 8/361; A61K 8/365; A61K 8/67; A61K 8/671; A61K 8/675; A61K 8/676; A61K 8/678; A61K 8/735; A61Q 19/08; G06Q 30/06211

(57) ABSTRACT

Provided herein are methods for providing customized skin regimen for a subject based on the levels of skin biomarkers and genetic statuses. The skin regimen includes suggestions for one or more of topicals, supplements, and/or life style changes for the subject.

### CROSS REFERENCE TO RELATED APPLICATIONS

This application is a continuation of PCT/US2024/053891, Filed Oct. 31, 2024, which claims the benefit of and priority to the U.S. Provisional Patent Application No. 63/546,671, filed on Oct. 31, 2023, the entire content of which is incorporated herein by reference in its entirety.

### BACKGROUND

Flawless skin is universally desired by people, as they are constantly on the quest to find the “best” skin products to achieve even-toned, clear, and radiant skin. This has led to an exponential rise of unverified advice or products to achieve good skin in the market. A person's skin health is dependent on factors such as susceptibility to skin irritants, allergies, immune function, genetic makeup, oxidative stress, collagen degradation, elastin degradation, and a variety of other factors. Commercial products have been created and developed to address common skin concerns such as pigmentation, acne, wrinkling, skin sagging, skin aging, etc., that correlate to the numerous skin health factors. However, although commercially available skin care products have attempted to cater to different skin types, these products generally lack the ability to improve the skin on personal level.

### SUMMARY

Disclosed herein are customized skin regimens that can help improve an individual's skin health based on a particular individual's skin profile. Such customized skin regimen can effectively provide personalized skin products, dietary, supplement, and lifestyle suggestions along with routine monitoring strategies in accordance an individual's skin profile. Further disclosed herein are methods for determining quantities of each active element to be included in the customized skin regimens.

In one aspect, provided herein is a method for determining a customized skin regimen for a subject, the method comprising:

A method for determining a customized skin regimen for a subject, the method comprising:
for one or more skin activity categories, each skin activity category comprising a plurality of elements being considered for inclusion in the customized skin regimen:
   i. obtaining or having obtained one or more of:
      genetic statuses of one or more genomic locations related to the skin activity category from one or more samples obtained from the subject;
      methylation statuses of one or more genes involved in biological processes of the skin activity category; and
      an image captured of the subject's skin;
   ii. determining a methylation score, a phenotype score, and a genetic score for the skin activity category for the subject using the one or more of genetic statuses, methylation statuses, and the image;
   iii. for each of one or more of the methylation score, phenotype score, and genetic score, determining a fixed value for each of the plurality of elements of the skin activity category using the corresponding score;
   iv. for each element of the plurality of elements, combining fixed values across the one or more of the methylation score, phenotype score, and genetic score to determine a percentage of the element to be included in the customized skin regimen.

In various embodiments, the one or more skin activity categories comprise an anti-aging and wrinkle reduction category, a lymphatic drainage category, a skin firming and elasticity category, a brightening and pigmentation category, a UV/oxidative damage/DNA/cell damage category, an anti-inflammatory and soothing category, and a hydration and skin barrier repair category. In various embodiments, determining the methylation score for the skin activity category for the subject comprises combining methylation levels of one or more genes in the skin activity category. In various embodiments, the skin activity category is the anti-aging and wrinkle reduction category, and wherein the one or more genes include one or more of CDKN2A, COL1A1, MMP1, and SIRT. In various embodiments, the skin activity category is the lymphatic drainage category and wherein the one or more genes include one or more of NFAT5, VEGFC, and PROX1. In various embodiments, the skin activity category is the skin firming and elasticity category and wherein the one or more genes include one or more of ELN, KLF4, and WRN. In various embodiments, the skin activity category is the brightening and pigmentation category and wherein the one or more genes include one or more of IGF2 and TYR. In various embodiments, the skin activity category is the UV/oxidative damage/DNA/cell damage category and wherein the one or more genes include one or more of TP53, BCL2, and SYN1. In various embodiments, the skin activity category is the anti-inflammatory and soothing category and wherein the one or more genes include one or more of TNF-a, CSCL8, and IL-6. In various embodiments, the skin activity category is the hydration and skin barrier repair category and wherein the one or more genes include one or more of FILAGGRIN (FLG), TGM1, CSCL8, CLDN1, and AQP3.

In various embodiments, combining methylation levels of one or more genes in the skin activity category is determined according to a formula: methylation age in each category=Σ(methylation level of gene×weight). In various embodiments, each weight for the one or more genes in the skin category is between 0.1 and 0.3. In various embodiments, determining the phenotype score for the skin activity category comprises determining one or more values by analyzing the image of the subject. In various embodiments, determining one or more values comprises manually analyzing the image for characteristics relevant to the skin activity category. In various embodiments, determining one or more values comprises applying an automated process to analyze the image for characteristics relevant to the skin activity category. In various embodiments, determining the phenotype score further comprises determining a second value based on answers included on a skin questionnaire including a set of questions related to the skin activity category. In various embodiments, the phenotype score for the skin activity category comprises combining the one or more values determined by analyzing the image and the second value determined based on answers included on the skin questionnaire. In various embodiments, determining the phenotype score further comprises mapping the combined one or more values and the second value to the phenotype score. In various embodiments, determining the genetic score comprises determining a quantity of genetic issues in the subject. In various embodiments, the quantity of genetic issues comprises one or more genetic statuses indicative of a mutation. In various embodiments, determining a fixed value for each of the plurality of elements comprises accessing a look up table. In various embodiments, at least two of the plurality of elements have different determined fixed values. In various embodiments, combining fixed values across the one or more of the methylation score, phenotype score, and genetic score comprises determining an average of fixed values of the methylation score and the phenotype score. In various embodiments, combining fixed values across the one or more of the methylation score, phenotype score, and genetic score further comprises summating the determined average of fixed values of the methylation score and the phenotype score and a fixed value of the genetic score. In various embodiments, combining fixed values across the one or more of the methylation score, phenotype score, and genetic score further comprises scaling the summated value to determine the percentage of the element to be included in the customized skin regimen.

In some embodiments, the plurality of elements for each skin activity category comprises one or more of peptides.

In some embodiments, the one or more of peptides for the anti-aging and wrinkle reduction category comprise one or more of palmitoyl pentapeptide-3 (Matrixyl), acetyl dipeptide-1 cetyl ester (Idealift), acetyl hexapeptide-8 (Argireline), acetyl hexapeptide-30 (Inyline®-Peptide), acetyl hexapeptide-51 amide (Juveleven), Argirelox (Pentapeptide 18+Argireline), SNAP 8-acetyl octapeptide-3, Tripeptide 5, GHK Cu, Reproage™, Synake, or palmitoyl pentapeptide 38.

In some embodiments, the one or more of peptides for the lymphatic drainage category comprise eyeseryl-acetyl tetrapeptide-5.

In some embodiments, the one or more of peptides for the skin firming and elasticity category comprise one or more of palmitoyl hexapeptide-12 (Biopeptide EL), palmitoyl oligopeptide (Bio-bustyl), myristoyl pentapeptide-8 (SymPeptide 225), palmitoyl oligopeptide (Biopeptide CL), acetyl heptapeptide 4 (Fensebiome), triptide-10 citrulline (Decorinyl), hexapeptide-10 (Serilesine), acetyl tetrapeptide-11 (Syniorage), acetyl hexapeptide-38 (Adifyline), hexapeptide-11 (Peptide Vinci 2), acetyl dipeptide-13 diphenylglycine (Relistase), palmitoyl dipeptide-5 diaminobutyloyl hydroxythreonine (and) palmitoyl dipeptide-6 diaminohydroxybutyrate (Syntacks), or acetyl tetrapeptide-2 (Uplevity).

In some embodiments, the one or more of peptides for the hydration and skin barrier repair category comprise one or more of Decapeptide-4, acetyl hexapeptide-37 (Diffuporine), Oligopeptide-24 (EGF-like peptide), FSS Ceramide 3, sodium pca, or APT-GPF.

In some embodiments, the one or more of peptides for the anti-inflammatory and soothing category comprise one or more of acetyl hexapeptide-49 (Delisens), palmitoyl tetrapeptide-7, acetyl tetrapeptide-15 (Skinasensyl), polyquaternium-51, allantoin, hesperdine methyl chalcone, acetyl tetrapeptide-40 (TELANGYN), acetyl dipeptide-3 aminohexanoate (Bodyfensine), Oligopeptide-10, or Acetyl Tetrapeptide-2 (Syn-UP).

In some embodiments, the one or more of peptides for the brightening and pigmentation category comprise one or more of Hexapeptide-2, Nonapeptide-1 (Melanostatine), Decapeptide 12, Oligopeptide-34, or Oligopeptide-51.

In some embodiments, the one or more of peptides for the UV/Oxidative Damage/DNA/Cell damage category comprise one or more of DAP tripeptide 33, pepha tight, ferulic acid, acetyl tetrapeptide-22 (Thermostressine), acetyl heptapeptide-1, or tripeptide-9 citrulline (dGlyage).

In some embodiments, the customized skin regimen further comprises one or more prebiotic and probiotic.

In some embodiments, the one or more prebiotic and probiotic comprise one or more of Pepha active, beta glucan, Bacillus extract, sea kelp extract, or alpaflor sebum.

In some embodiments, the customized skin regimen further comprises one or more vitamins.

In some embodiments, the one or more vitamins comprises one or more of D-Panthenol, magnesium ascorbyl phosphate, tocopherols, or niacinamide.

In some embodiments, the customized skin regimen further comprises one or more base elements.

In some embodiments, the method further comprises:
i. obtaining or having obtained levels of a plurality of skin biomarkers from one or more samples obtained from the subject;
ii. obtaining or having genetic statuses of one or more genomic locations of a plurality of genes from the one or more samples obtained from the subject; and
iii. determining the customized skin regimen for the subject using both the levels of the plurality of skin biomarkers and the genetic statuses of one or more genomic locations of a plurality of genes.

In some embodiments, the skin biomarkers comprise one or more of collagens and elastins obtained from serum of the subject.

In some embodiments, the collagen biomarkers comprise one or more of amino-terminal propeptide of Collagen 1 (PINP), carboxy-terminal propeptide of type 1 collagen (PICP), crosslinked telopeptide of type 1 collagen (ICTP), amino-terminal propeptide of collagen 3 (PIIINP), matrix metalloproteinases-1 (MMP-1), tumor necrosis factor-alpha (TNF-α), insulin-like growth factor-1 (IGF-1), or basic fibroblast growth factor (FGF-2).

In some embodiments, the elastin biomarkers comprise one or more of matrix metalloproteinases-12 (MMP-12) or elastin-derived peptides (EDPs).

In some embodiments, the serum is collected using one or more of ethylenediaminetetraacetic acid (EDTA), serum separator tube (SST), dried blood spot, or ficoll tube.

In some embodiments, the levels of the plurality of skin biomarkers are obtained by using one or more of microarray or mass spectrometry.

In some embodiments, the skin biomarkers comprise one or more lipids obtained from sebum of the subject.

In some embodiments, the sebum is obtained using an absorbent medium or material.

In some embodiments, the one or more lipids comprise one or more of glycerol lipids, free fatty acids, or cholesterol.

In some embodiments, the skin biomarkers from the one or more samples obtained from the subject comprise skin biomarkers from urine of the subject.

In some embodiments, the skin biomarkers from urine of the subject comprise one or more UV damage metabolites and oxidative stress loads from the urine of the subject.

In some embodiments, the skin biomarkers comprise one or more advanced glycation end products from urine or blood of the subject.

In some embodiments, the advanced glycation end products comprise one or more of carboxymethyl lysine (CEL), carboxymethyl lysine (CML), glucosepane, or pentosidine.

In some embodiments, the skin biomarkers comprise one or more microorganisms from skin niches of the subject.

In some embodiments, the microorganisms comprise one or more of Propionibacterium acnes, Propionibacterium avidum, or Propionibacterium granulosum, optionally from forehead of the subject.

In some embodiments, the microorganisms comprise Propionibacterium acnes, optionally from cheek of the subject.

In some embodiments, the microorganisms comprise one or more of Staphylococcus epidermidis, Corynebacterium spp, or Propionibacterium acnes, optionally from nasolabial fold of the subject.

In some embodiments, the microorganisms comprise Propionibacterium acnes, optionally from chin of the subject.

In some embodiments, the microorganisms comprise Firmicutes, optionally from face of the subject.

In some embodiments, the microorganisms comprise one or more of Corynebacterium kroppenstedtiin or Staphylococcus epidermidis, optionally from cheek of the subject.

In some embodiments, the microorganisms comprise Corynebacterium kroppenstedtiin, optionally from nasolabial fold of the subject.

In some embodiments, the microorganisms comprise Staphylococcus epidermidis, optionally from eyelid of the subject.

In some embodiments, the microorganisms comprise Staphylococcus epidermidis, optionally from cheek of the subject.

In some embodiments, the microorganisms comprise Corynebacterium species, optionally from chin of the subject.

In some embodiments, the microorganisms comprise Malassezia spp, optionally from cheek of the subject.

In some embodiments, the microorganisms comprise one or more Eikenella corrodens, Cutibacterium, Micrococcus luteus, or Kocuria, optionally from cheek of the subject.

In some embodiments, the microorganisms comprise Acinetobacter, optionally from cheek of the subject.

In some embodiments, the microorganisms comprise Corynebacterium, optionally from cheek of the subject.

In some embodiments, the microorganisms comprise one or more of Corynebacterium amycolatum, Proteobacteria, Firmicutes, Bacteroidetes, or Propionibacterium, optionally from forehead of the subject.

In some embodiments, the microorganisms comprise Propionibacterium, optionally from cheek of the subject.

In some embodiments, the microorganisms comprise one or more of Corynebacterium, Lactobacillus, or Cutibacterium, optionally from face of the subject.

In some embodiments, the skin biomarkers comprise one or more hormones from urine or blood of the subject.

In some embodiments, when the subject is a male, the hormones comprise one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG).

In some embodiments, when the subject is a male, obtaining the levels of a plurality of skin biomarkers further comprises determining one or more combinations of levels of the one or more hormones.

In some embodiments, determining one or more combinations of levels of the one or more hormones comprises determining one or more of Estriol (E3)/(E1+E2), 2-OH (E1+E2)/16a-OH E1, 2-MeO E1/2-OH E1, 4-MeO E1/4-OH E1, 4-MeO E2/4-OH E2, testosterone (T)/Epi-T, b-pregnanediol/E2, cortisol/cortisone, or metabolized cortisol (THF+THE), or Prostaglandin (Pg)/E2.

In some embodiments, when the subject is a female, the hormones comprise one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5α-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG).

In some embodiments, when the subject is a female, obtaining the levels of a plurality of skin biomarkers further comprises determining one or more combinations of levels of the one or more hormones.

In some embodiments, determining one or more combinations of levels of the one or more hormones comprises determining one or more of E3/(E1+E2), 2-OH (E1+E2)/16a-OH E1, 2-MeO E1/2-OH E1, 4-MeO E1/4-OH E1, 4-MeO E2/4-OH E2, T/Epi-T, b-pregnanediol/E2, cortisol/cortisone, or metabolized cortisol (THF+THE), or Prostaglandin (Pg)/E2.

In some embodiments, the skin biomarkers comprise one or more metabolism markers from serum or urine of the subject.

In some embodiments, the metabolism markers comprise one or more serum metabolism biomarkers.

In some embodiments, the serum metabolism biomarkers comprise one or more of lipid metabolism biomarkers, carbohydrate metabolism biomarkers, or cellular biomarkers.

In some embodiments, the lipid metabolism biomarkers comprise one or more of vaccenic acid, adiponectin, acylhexosylceramide (AHC), diacylglycerol (DAG), dimethyl-phosphatidylethanolamine (DMPE), or plasminogen activator inhibitor-1 (PAI-1).

In some embodiments, the carbohydrate metabolism biomarkers comprise one or more of adiponectin, gastric-inhibitor-peptide (GIP), or leptin.

In some embodiments, the cellular biomarkers comprise nicotinamide adenine dinucleotide (NAD).

In some embodiments, the metabolism markers comprise one or more urine metabolism biomarkers.

In some embodiments, the urine metabolism biomarkers comprise one or more of lipid metabolism biomarkers or carbohydrate metabolism biomarkers.

In some embodiments, the lipid metabolism biomarkers comprise one or more of adipate, suberate, or ethyl malonate.

In some embodiments, the carbohydrate metabolism biomarkers comprise one or more of pyruvate, L-lactate, or β-hydroxybutyrate.

In some embodiments, the skin biomarkers comprise one or more nutrients from the subject.

In some embodiments, the nutrients comprise one or more vitamins or minerals.

In some embodiments, the vitamins or minerals comprise one or more of Folate (L-5-methyltetrahydrofolate), magnesium, iron, vitamin A (retinol), vitamin D3 (cholecalciferol), vitamin E, choline, free Carnitine, vitamin B1 (thiamine diphosphate), vitamin B2 (riboflavin 5-Phosphate), vitamin B5 (pantothenic acid), vitamin B6 (pyridoxal 5-Phosphate), vitamin C (L-ascorbic acid), copper, zinc, selenium, chromium, or manganese.

In some embodiments, the nutrients comprise one or more vitamins and minerals, amino acids, or fatty acids.

In some embodiments, the vitamins and minerals comprise vitamin A (retinol), vitamin A (beta-carotene), vitamin B1 (thiamine diphosphate), vitamin B2 (riboflavin 5-phosphate), vitamin B3 (nicotinic acid), vitamin B5 (pantothenic acid), vitamin B6, pyridoxal 5-phosphate, vitamin B7 (biotin), vitamin B12 (cyanocobalamin), vitamin C (L-ascorbic acid), vitamin D (25-OH), vitamin D3 (cholecalciferol), vitamin D (1-25 dihydroxy), vitamin E (alpha-tocopherol), vitamin K1 (phylloquinone), vitamin K2 (menaquinone-MK-7), folate (L-5-methyltetrahydrofolate), coenzyme Q10 (ubiquinone+ubiquinol), selenium, sodium, potassium, calcium, zinc, manganese, iron, magnesium, copper, chromium, myoinositol, iodine, molybdenum, phosphorus, tetrahydrobiopterin, or fluoride, copper/zinc.

In some embodiments, the method further comprises further comprising determining one or more combinations of levels of one or more of the vitamins and minerals.

In some embodiments, determining one or more combinations of levels of one or more of the vitamins and minerals further comprises obtaining a ratio of copper/zinc.

In some embodiments, the amino acids comprise one or more of glutathione (oxidized), methylmalonic acid (MMA), choline, L-cysteine, L-asparagine, L-glutamine, L-serine, L-arginine, L-citrulline, L-isoleucine, L-valine, L-leucine, free carnitine, or phenylalanine.

In some embodiments, the fatty acids comprise one or more of docosahexaenoic acid (DHA), Eicosapentaenoic acid (EPA), docosapentaenoic acid (DPA), arachidonic acid (AA), or linoleic acid (LA).

In some embodiments, the method further comprises obtaining the levels of a plurality of skin biomarkers by determining one or more combinations of levels of one or more of the fatty acids.

In some embodiments, determining one or more combinations of levels of one or more of the fatty acids further comprises obtaining omega-3 total, omega-6 total, omega-3 index, or a ratio of AA/EPA.

In some embodiments, the skin biomarkers comprise one or more commensal microorganisms from the subject.

In some embodiments, the commensal microorganisms comprise one or more of commensal bacteria, archaea, fungi, or viruses.

In some embodiments, the skin biomarkers further comprise gut metabolites from the subject.

In some embodiments, the method further comprises obtaining or having obtained one or more demographics of the subject; and determining the customized skin regimen for the subject based on the demographics of the subject.

In some embodiments, the demographics comprise one or more of an age or gender of the subject.

In some embodiments, the method further comprises:
i. obtaining or having obtained an image of a face from the subject;
ii. grading severities of one or more phenotypic parameters for the face of the subject based on the image; and
iii. determining the customized skin regimen for the subject based on the severity of the one or more phenotypic parameters.

In some embodiments, the phenotypic parameters comprise one or more of wrinkles, periorbital-around eyes (puffy eyes), nasolabial folds (laugh lines), worry lines, uneven skin tone/pigmentation, or age spots (liver spots).

In some embodiments, the method further comprises obtaining or having obtained answers to a skin questionnaire from the subject; and determining the customized skin regimen for the subject based on the answers from the subject.

In some embodiments, the questionnaire comprises one or more questions related to skin goals, type of skin, sun exposure, or skin proneness to external factors.

In some embodiments, the method further comprises administering the customized skin regimen to the subject.

In another aspect, provided herein are customized skin regimens, wherein the customized skin regimen is determined according to a method disclosed herein.

In some embodiments,
i. a skin biomarker is the Amino terminal propeptide of Collagen 1 (PINP), and the composition comprises one or more of vitamin C, peptides, retinoids, copper peptides, copper, collagen or collagen peptides, and/or omega-3 fatty acids;
ii. a skin biomarker is the Carboxy terminal propeptide of type 1 collagen (PICP) and the composition comprises one or more of hyaluronic acids, retinoids, vitamin C, niacinamide, collagen or collagen peptides, zinc, and/or omega-3 fatty acids;
iii. a skin biomarker is the Crosslinked telopeptide of type 1 collagen (ICTP), and the composition comprises one or more of retinoids, vitamin C, peptides, proline, glycine, vitamin E, vitamin C, and/or copper;
iv. a serological skin biomarker is the Amino terminal propeptide of Collagen 3 (PIIINP), and the composition comprises one or more of alpha hydroxy acids, retinoids, vitamin C, peptides, glycine, proline, and/or omega-3 fatty acids;
v. a skin biomarker is the Matrix metalloproteinase-1 (MMP-1) and composition comprises one or more of ginseng, green tea extract, retinoids, vitamin C, peptides, collagen or collagen peptides, vitamin E, vitamin C, and/or omega-3 fatty acids;
vi. a skin biomarker is Insulin like growth factor-1 (IGF-1) and the composition comprises one or more of capsaicin, vitamin C, hyaluronic acid, zinc, vitamin C, probiotics, collagen or collagen peptides, and/or omega-3 fatty acids;
vii. a skin biomarker is Basic fibroblast growth factor (FGF-2) and the composition comprises one or more of ginseng, vitamin C, hyaluronic acid, collagen or collagen peptides, resveratrol, glycine, and/or proline;
viii. a skin biomarker is Matrix Metalloproteinase-12 (MMP-12) and the composition comprises one or more of vitamin C, green tea extract, silicon, ginseng, and/or vitamin E;
ix. a skin biomarker is Elastin derived peptides (EDPs), and the composition comprises one or more of niacinamide, hyaluronic acid, silicon, vitamin A, and/or aloe vera.

In some embodiments, the peptide is one or more of Hexapeptide-11, GHK-Cu, Palmitoyl Pentapeptide-4 (Pal-KTTKS), Palmitoyl Tetrapeptide-7 (Pal-GQPR), Dipeptide-2, Palmitoyl Pentapeptide-3, decapeptide-12, Dipeptide Diaminobutyroyl Benzylamide Diacetate, Palmitoyl Dipeptide-5 Diaminobutyroyl Hydroxythreonine, Palmitoyl Dipeptide-5 Diaminohydroxybutyrate, Tetradecyl Aminobutyroylvalylaminobutyric Urea Trifluoroacetate, Acetyl Tetrapeptide-5, Heptapeptide-4, Acetyl Hexapeptide-51 Amide, Acetyl Hexapeptide-30, Acetyl Hexapeptide-8 (Argireline), acetyl hexapeptide-3 (Argireline), Acetylarginyltryptophyl Diphenylglycine, Acetyl Hexapeptide-37, Hexapeptide-10, Acetyl Octapeptide-3, Tetrapeptide-1, Tetrapeptide-2, Acetyl Tetrapeptide-2, Acetyl Hexapeptide-38, Acetyl Dipeptide-3 Aminohexanoate, Tripeptide-10 Citrulline (Decorinyl), Acetyl Hexapeptide-49, Pentapeptide 18, Tripeptide 29, Nonapeptide-1, Tripeptide 5, Palmitoyl Tripeptide-5, Diaminopropionoyl (DAP) Tripeptide-33, beta glucan, and/or bacillus extract.

In some embodiments, the retinoid is one or more of retinol, retinal, tretinoin (retinoic acid), isotretinoin, alitretinoin, adapalene, bexarotene, tazarotene, and/or Trifarotene.

In some embodiments, the omega-3 fatty acid is one or more of Alpha-linolenic acid (ALA), Eicosapentaenoic acid (EPA), and/or Docosahexaenoic acid (DHA).

In some embodiments, the Alpha hydroxy acid is one or more of glycolic acid, lactic acid, mandelic acid, and/or citric acid.

In some embodiments, wherein
i. a polymorphism is a SNP associated with melanin synthesis and the composition comprises one or more of Hydroquinone, Retinoids, Azelaic acid, Kojic acid, Cysteamine, Lignin peroxidase, Niacinamide, Ellagic acid, L-Cystine, L-Glutathione, Polypodium leucotomos, Melatonin, Arbutin, and/or Vitamin C;
ii. a polymorphism is a SNP associated with Moisturization and the composition comprises one or more of Vitamin E, Coenzyme Q10, Vitamin F, Kumazasa extract, Magnesium ascorbyl phosphate, Hyaluronic acid, Magnesium oil, collagen, ceramides, hyaluronan, procyanidin, probiotics, Galactooligosaccharides, Alpha-linolenic acid, and/or Gamma-linolenic acid;
iii. a polymorphism is a SNP associated with Skin Inflammation and the composition comprises one or more of Coenzyme Q10, Methylsulfonylmethane, Retinoid, Vitamin E, Vitamin C, Curcumin, Green tea or green tea extract, and/or Omega 3 fatty acids;
iv. a polymorphism is a SNP associated with Melanocyte damage and the composition comprises one or more of Coenzyme Q10, Methylsulfonylmethane, Retinoid, Vitamin E, Vitamin D, Zinc, Vitamin C, Curcumin, Green tea or green tea extract, and/or Omega 3 fatty acids;
v. a polymorphism is a SNP associated with Collagen degradation and the composition comprises one or more of Retinoids, Vitamin C, Vitamin E, Tretinoin, Collagen peptides, Red ginseng, Aloe vera extract, Hyaluronic acid, Ginkgo biloba, and/or Soy;
vi. a polymorphism is a SNP associated with Elastin synthesis and the composition comprises one or more of Retinoid, Vitamin C, Collagen or collagen peptides, Hyaluronic acid, Dexpanthenol, Vitamin E, Vitamin A, and/or Zinc;
vii. a polymorphism is a SNP associated with Antioxidants and the composition comprises one or more of Vitamin C, Coenzyme Q10, Polyphenols, Vitamin E, Curcumin, Vitamin C, Vitamin E, Beta carotene, Flavanols, zinc, Lutein, Lycopene, Silymarin, and/or Green tea or green tea extract;
viii. a polymorphism is a SNP associated with Vitamin A deficiency and the composition comprises one or more of Retinol, Vitamin E, Resveratrol, Baicalin, B-vitamins, Vitamin C, Epigallocatechin gallate, Collagen, Nicotinamide riboside, Nicotinamide mononucleotide, Coenzyme Q10, and/or Curcumin;
ix. a polymorphism is a SNP associated with Vitamin B6 deficiency and the composition comprises one or more of B-vitamins, Vitamin B6, Coenzyme Q10, Methylsulfonylmethane; Retinoids, and Vitamin E, Curcumin, Green tea or green tea extract, Omega 3 fatty acids, and/or Vitamin C;
x. a polymorphism is a SNP associated with Vitamin B12 deficiency and the composition comprises one or more of Retinol, Vitamin E, Resveratrol, Baicalin, B-vitamins, Vitamin B12, Vitamin C, Curcumin, Epigallocatechin gallate, Collagen, Nicotinamide riboside, Nicotinamide mononucleotide, and/or Coenzyme Q10;
xi. a polymorphism is a SNP associated with Vitamin C deficiency and the composition comprises one or more of Vitamin C, Coenzyme Q10, Polyphenols, Vitamin E, Curcumin, Beta carotene, Flavanols, zinc, Lutein, Lycopene, Silymarin, and/or Green tea or green tea extract;
xii. a polymorphism is a SNP associated with Vitamin D deficiency and the composition comprises one or more of Vitamin D, Retinol, Vitamin E, Resveratrol, Baicalin, B-vitamins, Vitamin C, Curcumin, Epigallocatechin gallate, Collagen, Nicotinamide riboside, Nicotinamide mononucleotide, and/or Coenzyme Q10;
xiii. a polymorphism is a SNP associated with Folate deficiency and the composition comprises one or more of Folate, Retinol, Vitamin E, Resveratrol, Baicalin, B-vitamins, Vitamin C, Curcumin, Epigallocatechin gallate, Collagen, Nicotinamide riboside, Nicotinamide mononucleotide, and Coenzyme Q10; and/or
xiv. a polymorphism is a SNP associated with Omega fatty acid deficiency and the composition comprises one or more of DHA (Docosahexaenoic acid), EPA, Coenzyme AL, Methylsulfonylmethane, Retinoid, Curcumin, Green tea or green tea extract and/or Vitamin E.

In some embodiments, a phenotypic parameter is wrinkles, Periorbital-around eyes (puffy eyes), Nasolabial folds (Laugh lines), worry lines, Uneven Skin tone/Pigmentation, and/or Age spots (Liver spots) and the composition comprises one or more of Tretinoin, Zeaxanthin, Retinol, Retinoids, Vitamin C, Hyaluronic acid, Caffeine, Phytonadione, Vitamin E, Astaxanthin, Hyaluronic acid, Peptides, Niacinamide, Azelaic acid, Soybean extract, Kojic acid, Aloesin, and/or Turmeric.

In some embodiments, the customized skin regimen further comprises one or more of Zeaxanthin, Collagen, Vitamin C, Resveratrol, Vitamin E, Vitamin C, Hyaluronic acid, Glutathione, Niacinamide, and/or Coenzyme Q10.

In some embodiments, a skin questionnaire question is:
i. UV and the composition comprises one or more of Zinc oxide, titanium oxide, Polypodium leucotomos, Vitamin E, Vitamin C, and/or Niacinamide;
ii. Anti-wrinkling and the composition comprises one or more of Tretinoin, Zeaxanthin, Retinol, Vitamin C, Hyaluronic acid, Collagen, Resveratrol, and/or Vitamin E;
iii. Anti-pigmentation and the composition comprises one or more of Niacinamide, Azelaic acid, Vitamin C, Soybean extract, Retinoids, Kojic acid, Vitamin E, Aloesin, Turmeric, and/or Glutathione
iv. Moisturization and the composition comprises one or more of Hyaluronic acid, Glycerin, Shea butter, Jojoba oil, Hyaluronan, Ceramides, and Collagen;
v. Radiant complexion and the composition comprises one or more of Vitamin C and/or Niacinamide, vi. Dry skin and the composition comprises Glycerin;
vii. Combinational skin and the composition comprises Hyaluronic acid;
viii. Acne-prone skin and the composition comprises Benzoyl peroxide;
ix. Sun exposure and the composition comprises Zinc oxide and/or Titanium dioxide;
x. Sensitive skin—stinging or burning and the composition comprises Niacinamide
xi. Sun and Humidity and the composition comprises one or more of Vitamin E, Zinc oxide, Astaxanthin, and/or Omega-3 fatty acids
xii. Wind and cold and the composition comprises one or more of Petroleum jelly, Omega-3 fatty acids, and/or Probiotics.

In some embodiments, a hormone is:
i. one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG); or
ii. one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5α-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG); and
iii. the composition comprises one or more of Calcium D-glucarate, Green Tea, Maca DIM (3,3′-diindolylmethane), Black Cohosh, Ginger, Cranberry, Milk Thistle, Hops, Red clover, Damiana, Chaste Tree Berry Extract, Vitamin B2, Fenugreek seed, Ashwagandha, Vitamin D3, Zinc, Tribulus, Shatavari, Zinc, Shatavari, DHA, EPA, pine nuts, Licorice Root Extract, Oregano, Curcumin, Sodium, Rhodiola rosea, Vitamin B6, Vitamin B12, Magnesium, Folate, Iodine, Selenium, Potassium, Magnesium, Melatonin, Huang-qin, St. John's-wort, SAMe (S-adenosyl methionine), and/or Trimethylglycine (betaine).

In some embodiments, a metabolic biomarker is one or more of Vaccenic acid, Adiponectin, Acylhexosylceramide (AHC), Dimethyl-phosphatidylethanolamine (DMPE), PAI-1, GIP, Leptin, NAD, Adipate, Suberate, Ethyl malonate, Pyruvate, L-lactate, and/or β-hydroxybutyrate and the supplemental or topical is one or more of Conjugated Linoleic Acid, DHA, EPA, Vitamin D, Curcumin, Berberine Extract, Resveratrol, Vitamin B3, Vitamin B6, Vitamin E, Choline, Vitamin C, Tryptophan, Nicotinamide riboside, Resveratrol, Quercetin, Vitamin B2, L-carnitine, Vitamin B1, Vitamin B12, CoQ10 (Coenzyme Q10), Alpha lipoic acid, and/or Chromium.

In some embodiments, the customized skin regimen further comprises an excipient or carrier.

In some embodiments, the excipient or carrier is one or more of water, Caprylyl Glycol, butylene glycol, ultra low molecular weight (ulmw) hyaluronic acid, middle molecular weight (MMW) hyaluronic acid, and/or dimethyl isosorbide

In another aspect, provided herein are customized skin regimens, wherein the customized skin regimen comprises one or more supplement or topical medication, or a supplement and a topical medication.

In some embodiments, the one or more topical medication and/or supplement comprise one or more of B-vitamins, Vitamin B1, Vitamin B2, Vitamin B3, Vitamin B6, Vitamin B12, vitamin C, vitamin D, vitamin E, vitamin F, peptides, retinoids, copper peptides, copper, zinc, collagen or collagen peptides, omega-3 fatty acids, hyaluronic acids, niacinamide, zinc, proline, glycine, vitamin E, alpha hydroxy acids, ginseng, green tea or green tea extract, capsaicin, zinc, probiotics, resveratrol, glycine, proline, green tea or green tea extract, silicon, niacinamide, silicon, vitamin A, aloe vera, Hydroquinone, Azelaic acid, Kojic acid, Cysteamine, Lignin peroxidase, Ellagic acid, L-Cystine, L-Glutathione, Polypodium leucotomos, Melatonin, Arbutin, Coenzyme Q10, Vitamin F, Kumazasa extract, Magnesium ascorbyl phosphate, Magnesium oil, ceramides, hyaluronan, procyanidin, probiotics, Galactooligosaccharides, Alpha-linolenic acid, Gamma-linolenic acid, Methylsulfonylmethane, Curcumin, Red ginseng, Aloe vera extract, Dexpanthenol, Polyphenols, Beta carotene, Flavanols, zinc, Lutein, Lycopene, Silymarin, Baicalin, Epigallocatechin gallate, Nicotinamide riboside, Nicotinamide mononucleotide, Zeaxanthin, Caffeine, Phytonadione, Astaxanthin, Soybean extract, Aloesin, Turmeric, Glycerin, Shea butter, Jojoba oil, Benzoyl peroxide, Conjugated Linoleic Acid, Berberine Extract, Quercetin, Calcium D-glucarate, Zinc oxide and/or Titanium dioxide.

In some embodiments, the peptide is one or more of Hexapeptide-11, GHK-Cu, Palmitoyl Pentapeptide-4 (Pal-KTTKS), Palmitoyl Tetrapeptide-7 (Pal-GQPR), Dipeptide-2, Palmitoyl Pentapeptide-3, decapeptide-12, Dipeptide Diaminobutyroyl Benzylamide Diacetate, Palmitoyl Dipeptide-5 Diaminobutyroyl Hydroxythreonine, Palmitoyl Dipeptide-5 Diaminohydroxybutyrate, Tetradecyl Aminobutyroylvalylaminobutyric Urea Trifluoroacetate, Acetyl Tetrapeptide-5, Heptapeptide-4, Acetyl Hexapeptide-51 Amide, Acetyl Hexapeptide-30, Acetyl Hexapeptide-8 (Argireline), acetyl hexapeptide-3 (Argireline), Acetylarginyltryptophyl Diphenylglycine, Acetyl Hexapeptide-37, Hexapeptide-10, Acetyl Octapeptide-3, Tetrapeptide-1, Tetrapeptide-2, Acetyl Tetrapeptide-2, Acetyl Hexapeptide-38, Acetyl Dipeptide-3 Aminohexanoate, Tripeptide-10 Citrulline (Decorinyl), Acetyl Hexapeptide-49, Pentapeptide 18, Tripeptide 29, Nonapeptide-1, Tripeptide 5, Palmitoyl Tripeptide-5, Diaminopropionoyl (DAP) Tripeptide-33, beta glucan, and/or bacillus extract.

In some embodiments, the retinoid is one or more of retinol, retinal, tretinoin (retinoic acid), isotretinoin, alitretinoin, adapalene, bexarotene, tazarotene, and/or Trifarotene.

In some embodiments, the omega-3 fatty acid is one or more of Alpha-linolenic acid (ALA), Eicosapentaenoic acid (EPA), and/or Docosahexaenoic acid (DHA).

In some embodiments, the Alpha hydroxy acid is one or more of glycolic acid, lactic acid, mandelic acid, and/or citric acid.

In some embodiments, the customized skin regimen further comprises an excipient or carrier.

In some embodiments, the excipient or carrier is one or more of water, Caprylyl Glycol, butylene glycol, ultra low molecular weight (ulmw) hyaluronic acid, middle molecular weight (MMW) hyaluronic acid, and/or dimethyl isosorbide.

### BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 depicts a system overview for determining a customized skin regimen for an individual, in accordance with an embodiment.

FIG. 2A provides a diagram showing the information used to determine a customized skin regimen, in accordance with a first embodiment.

FIG. 2B provides a diagram showing the information used to determine a customized skin regimen, in accordance with a second embodiment.

FIG. 3 provides a flowchart of various exemplary methods for determining a customized skin regimen for an individual, in accordance with an embodiment.

FIG. 4 illustrates an example computing device for implementing system and methods described in FIGS. 1-3.

### DETAILED DESCRIPTION

### Definitions

Terms used in the claims and specification are defined as set forth below unless otherwise specified.

Abbreviations used in this application include the following:

The term “skin biomarkers” refers to any biomarkers that indicate the skin health of a subject.

The term “topical” is a medication pertaining to the skin, such as a topical anti-infective agent applied to a certain area of skin and affecting only the area to which it is applied. The topical or topical medication includes a large range of classes including creams, foams, gels, lotions, and ointments to treat ailments of the skin.

The term “supplement” is a product taken to supplement a person's diet.

The term “skin product” is any product intended to cleanse or beautify the skin. Depending on its purpose and effect, it can be cosmetics or medications.

The term “ameliorating” refers to any therapeutically beneficial result in the treatment of a disease state, e.g., a skin aliment, lessening in the severity or progression, remission, or cure thereof.

The term “sufficient amount” means an amount sufficient to produce a desired effect, e.g., an amount sufficient to change skin health condition of a subject.

The term “therapeutically effective amount” is an amount that is effective to ameliorate a symptom of a disease. A therapeutically effective amount can be a “prophylactically effective amount” as prophylaxis can be considered therapy.

The phrase “obtaining or having obtained” encompasses the active step of obtaining certain information and/or a step of instructing e.g., a third party to obtain the information.

It must be noted that, as used in the specification and the appended claims, the singular forms “a,” “an” and “the” include plural referents unless the context explicitly dictates otherwise.

### Overview

Due to people's awareness of good skin health, skin products have been massively increased but still cannot fill the gap in improving skin health on an individual level. To address the unique needs of an individual's skin, various elements that affect the skin's properties, such as genetic factors, immune function, oxidative stress, collagen degradation, elastin degradation and so on can be investigated for purposes of determining a customized skin regimen.

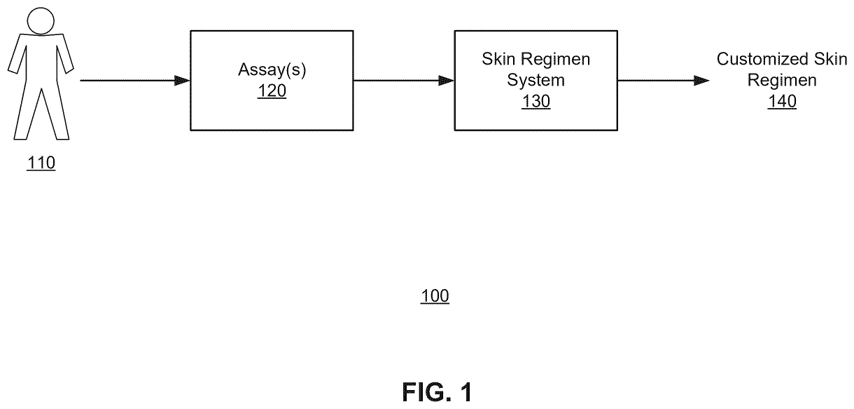

Reference is made to FIG. 1 which depicts a system overview for determining a customized skin regimen for an example individual 110, in accordance with an embodiment. FIG. 1 introduces an individual 110 who may undergo one or more assay(s) 120 to determine factors that are informative for determining a customized regimen. As described herein, example factors include one or more of serological skin biomarkers, sebum biomarkers, urology biomarkers, genetics, skin glycation, skin microbiome, individual's demographics, phenotypic skin appearance, and concerns of the individual (e.g., as determined through a skin questionnaire), and possible other factors (e.g., hormones, metabolism, nutrients, and gut microbiome, etc.).

Genetics is highly implicated in skin aging. Skin aging is a gradual phenomenon characterized by wrinkling, loss of elasticity, laxity, rough-textured appearance, etc. Skin aging is based on both intrinsic and extrinsic factors. Intrinsic skin aging (e.g., chronological aging) may be caused by genetics, cellular metabolism, hormones, and metabolic processes. This process is mainly characterized by functional alterations rather than by overall morphological changes. For example, dry and pale skin with fine wrinkles may indicate a certain degree of laxity and a variety of benign neoplasms. Exogenous or extrinsic factors affecting skin aging may include chronic light exposure, pollution, ionizing radiation, chemicals, toxins, and lifestyle factors (e.g., smoking, sleeping patterns, diet, and daily skincare habits). Extrinsic skin aging is also referred to as photoaging with visible manifestations including coarse wrinkles, solar elastosis, pigment irregularities, etc. Contrary to intrinsic skin aging, extrinsic skin aging is characterized by prominent morphologic and physiologic changes and may result in premature aging of the skin. Extrinsic aging accounts for about 80% of visible skin aging with the signs superimposing the signs of intrinsic skin aging at chronically exposed areas of the body.

The sign of wrinkles in extrinsic skin aging or photoaging relates to the weakening bond between the dermis and epidermis, which may be caused by marked loss of fibrillin-positive structures and reduced content of collagen type VII (Col-7). The distribution of collagen is altered in older skin with an increased ratio of Col-3 to Col-1. Glycosaminoglycans (GAGs), the primary moisture-retaining dermal skin matrix constituents, may be associated with abnormal elastotic material and thus be unable to function effectively. Assessing these fundamental components may help with understanding the status of an individual's skin. Genetic polymorphisms influence genes that are involved in melanocyte pigmentation, skin tanning response, hydration, collagen and elastin synthesis, and skin inflammation, all of which are important factors affecting the skin. Therefore, assessing genetic polymorphisms is an important determinant of phenotypic variation.

Lifestyle factors such as nutrient intake also play a major role in skin health. Nutritional antioxidants including vitamin C, vitamin E, carotenoids, copper, and selenium may promote skin health by scavenging free radicals. For example, free radicals generated from endogenous metabolic pathways or exogenous factors may lead to oxidative stress, where the exogenous factors include at least exposure to UV radiation, ionizing radiation, pollution, alcohol intake, or smoking. Oxidative stress results in the loss of functional characteristics and regenerative potential, exacerbating skin aging. Sleep routine is another important factor affecting skin health since good sleep routines contribute to lower trans-epidermal water loss, better skin recovery, and lower intrinsic skin aging scores.

Given that various endogenous and exogenous factors are affecting skin conditions, the present disclosure proposes methods and protocols that comprehensively assess skin health using skin-specific serum/urine biomarkers, genetic predispositions, and other various intrinsic and extrinsic aging factors to create personalized skin products and establish a personalized skincare regimen (including dietary and lifestyle interventions), thereby effectively improving an individual's skin health.

In some embodiments, the methods described herein measure and evaluate genetic, serum, and urine biomarkers that reflect the status of an individual's skin health using a ‘Skin Plus’ panel. These markers are tested using various applicable microarray or mass-spectrometry techniques. The test panel also assesses genetic polymorphisms that govern melanocyte pigmentation, skin tanning response, hydration, collagen and elastin synthesis, skin inflammation, etc. In some embodiments, genetic testing may be conducted using reverse transcription polymerase chain reaction. Overall, the ‘Skin Panel’ can assess the status of the individual's skin along with the genetic susceptibility to various skin aging factors.

In some embodiments, a ‘Skin Health’ questionnaire is also designed to gauge the individual's skin concerns, lifestyle factors, health comorbidities, and skincare goals. The methods provided herein take the results from the biomarker and genetics tests along with the inferences from the questionnaire to establish a personalized skin regimen. The methods may include creating personalized skin products, dietary and lifestyle suggestions, skincare suggestions, and recommendations for regular skin monitoring strategies.

In some embodiments, the methods disclosed herein include further tests to comprehensively assess the individual's intrinsic aging factors. In some embodiments, the methods disclosed herein apply ‘MetabolicPro’ and ‘HormonePro’ to test cellular metabolism and hormone health, respectively. In some embodiments, the present disclosure may also apply ‘NutriPro’ or ‘NutriLite’ tests to determine the influence of nutrient intake and nutrient deficiencies on the individual's skin health. In some embodiments, the present disclosure may further assess the gut microbiome to determine how the gut affects the skin via the ‘gut-skin axis’. The results from these further tests also can be considered in designing the personalized skin regimen.

FIG. 1 further introduces a skin regimen system 130. Generally, the skin regimen system 130 analyzes the various factors and determines a customized skin regimen 140 that is personalized for the individual 110. Example compositions of customized skin regimens are disclosed herein. In various embodiments, the customized skin regimen is provided to the individual 110. For example, the individual 110 can self-apply the customized skin regimen e.g., topically.

### Example Factors for Determining Customized Skin Regimen

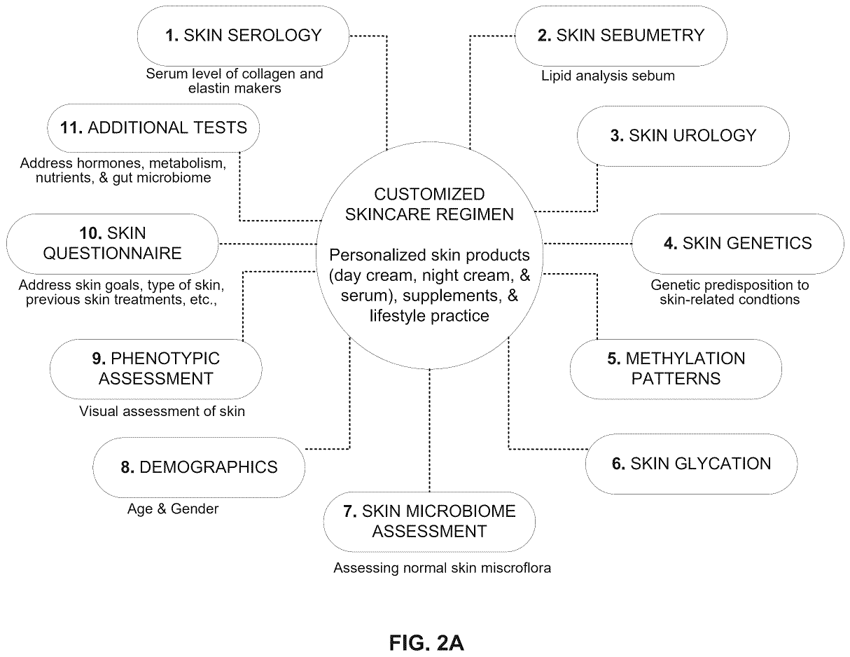

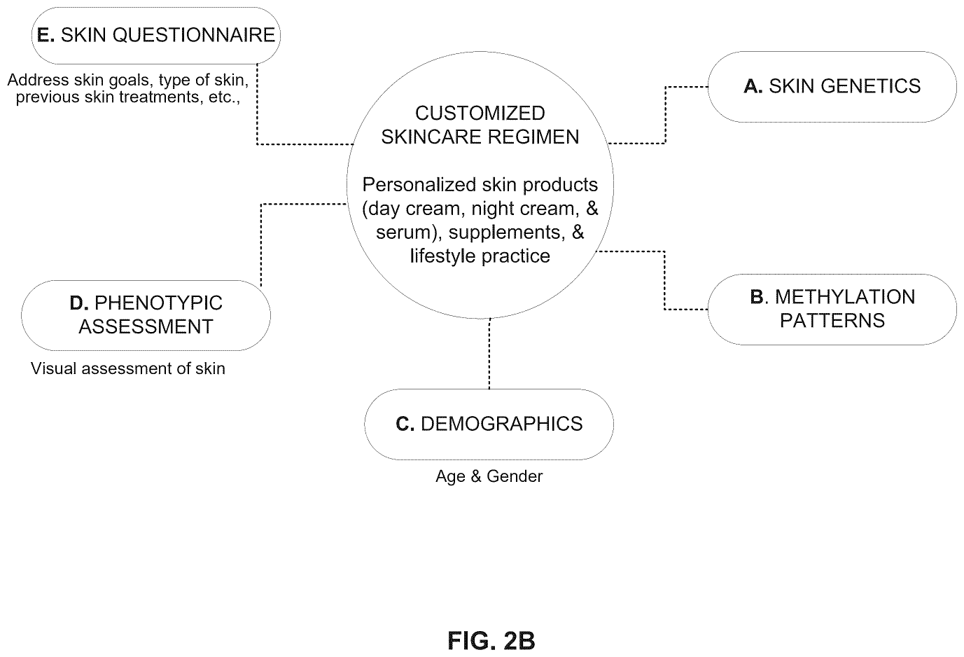

FIG. 2A provides an overview of the various aspects of skin biomarker testing, genetic testing, and other tests provided by the method and compositions disclosed herein. To customize the skincare routine per individual, the present disclosure may carry out the following analysis and procedures: (1) skin serology (e.g., serological analysis for serum levels of collagen and elastin), (2) skin sebumetry (e.g., sebumetry analysis for skin lipids), (3) skin urology (e.g., urine analysis for UV damage metabolites and oxidative stress load), (4) skin genetics (e.g., genetic analysis for skin parameters), (5) methylation patterns, (6) skin glycation, (7) skin microbiome assessment, (8) demographics, (9) phenotypic assessment, and (10) skin questionnaire information (e.g., experienced symptoms from the skin questionnaire), (11) additional tests including information regarding hormone levels, metabolism levels, nutrient levels, and gut microbiome.

In various embodiments, the analysis and procedure of (1) skin serology generates data relating to serological skin biomarkers. In various embodiments, the analysis and procedure of (2) skin sebumetry generates data relating to sebum biomarkers. In various embodiments, the analysis and procedure of (3) skin urology generates data relating to urology biomarkers. In various embodiments, the analysis and procedure of (4) skin genetics generates data relating to genetic testing (e.g., genetic information relating to genetic statuses of one or more genomic locations). In various embodiments, the analysis and procedure of (5) epigenetics generates data relating to methylation patterns. In various embodiments, the analysis and procedure of (6) skin glycation generates data relating to skin glycation. In various embodiments, the analysis and procedure of (7) skin microbiome assessment generates data relating to the skin microbiome assessment. In various embodiments, the analysis and procedure of (8) demographics generates data relating to demographics. In various embodiments, the analysis and procedure of (9) phenotypic assessment generates data relating to phenotypic assessment. In various embodiments, the analysis and procedure of (10) skin questionnaire generates data or information obtained via the skin questionnaire.

In various embodiments, methods for determining a customized skin regimen may include carrying out one or more of, two or more of, three or more of, four or more of, five or more of, six or more of, seven or more of, eight or more of, nine or more of, or ten or more of the following analysis and procedures: (1) skin serology (e.g., serological analysis for serum levels of collagen and elastin), (2) skin sebumetry (e.g., sebumetry analysis for skin lipids), (3) skin urology (e.g., urine analysis for UV damage metabolites and oxidative stress load), (4) skin genetics (e.g., genetic analysis for skin parameters), (5) methylation patterns, (6) skin glycation, (7) skin microbiome assessment, (8) demographics, (9) phenotypic assessment, and (10) skin questionnaire information (e.g., experienced symptoms from the skin questionnaire), (11) additional tests including information regarding hormone levels, metabolism levels, nutrient levels, and gut microbiome.

FIG. 2B provides a diagram showing the information used to determine a customized skin regimen, in accordance with a second embodiment. Here, FIG. 2B shows a reduced number of analysis and procedures in comparison to those shown in FIG. 2A. Here, the analysis and procedures of FIG. 2B include (A) skin genetics (e.g., genetic analysis for skin parameters), (B) methylation patterns, (C) demographics, (D) phenotypic assessment, and (E) skin questionnaire information (e.g., experienced symptoms from the skin questionnaire). In various embodiments, methods for determining a customized skin regimen may include carrying out one or more of, two or more of, three or more of, four or more of, or all five of the following analysis and procedures: (A) skin genetics (e.g., genetic analysis for skin parameters), (B) methylation patterns, (C) demographics, (D) phenotypic assessment, and (E) skin questionnaire information (e.g., experienced symptoms from the skin questionnaire). In various embodiments, methods for determining a customized skin regimen may include carrying out one or more of, two or more of, three or more of, or all four of the following analysis and procedures: (A) skin genetics (e.g., genetic analysis for skin parameters), (B) methylation patterns, (C) phenotypic assessment, and (E) skin questionnaire information (e.g., experienced symptoms from the skin questionnaire).

### Serological Skin Biomarkers

In one aspect, provided herein are methods of determining a level of skin biomarkers from serum of a subject (i.e., serological skin biomarkers).

In one aspect, provided herein are methods of providing a customized skin regimen based at least on the serological skin biomarkers of the subject, the method comprising: obtaining or having obtained levels of a plurality of skin biomarkers from one or more samples obtained from the subject, and determining the customized skin regimen for the subject using at least the plurality of skin biomarkers, where the skin biomarkers are the serological skin biomarkers.

In some embodiments, the serological skin biomarkers (or simply serological markers) comprise one or more collagens and elastins. In some embodiments, the collagen biomarkers comprise one or more of amino-terminal propeptide of Collagen 1 (PINP), carboxy-terminal propeptide of type 1 collagen (PICP), crosslinked telopeptide of type 1 collagen (ICTP), amino-terminal propeptide of collagen 3 (PIIINP), matrix metalloproteinases-1 (MMP-1), tumor necrosis factor-alpha (TNF-α), insulin-like growth factor-1 (IGF-1), or basic fibroblast growth factor (FGF-2). In some embodiments, the elastin biomarkers comprise one or more of matrix metalloproteinases-12 (MMP-12) or elastin-derived peptides (EDPs).

In some embodiments, the one or more of collagens and elastins are selected from Table 1. One or more tests are conducted for determining the levels of the one or more of collagens and elastins of a subject selected from Table 1.

Table 1 provides an exemplary set of serological markers that can be tested, along with their significance. The collagen biomarkers are synthesis and degradation biomarkers, and the elastin biomarkers are degradation biomarkers. For example, as shown in Table 1, determining a level of PINP can be used to improve collagen synthesis, and ICTP is measured to indicate collagen degradation, while MMP-12 and EDPs are tested to show the condition of skin elastin degradation. Both the collagen biomarkers and the elastin biomarkers have the potential to enhance the assessment of skin statuses and inform personal decision-making in skin treatment.

In some embodiments, the methods provided herein assess the levels of serological skin biomarkers in subject samples via any detection or collection method known in the art. In some embodiments, the detection methods include microarray and mass spectrometry. In some embodiments, the collection methods include ethylenediaminetetraacetic acid (EDTA), serum separator tube (SST), dried blood spot, or ficoll tube based methods.

The methods provided herein evaluate the serological skin biomarkers and provide a customized skin regimen. In some embodiments, the skin regimen includes suggestions of both topicals and supplements for each serological marker being tested. Table 2 shows exemplary suitable topical and supplement forms based on the testing of various serological markers.

A combination of topicals and/or supplements can be applied to improve skin health based on the determined levels of each serological biomarker. For example, based on the level of PINP tested for a specific subject, one or more of vitamin C, peptides, retinoids, and copper peptides may be applied on a particular area of the skin as topical medications, and one or more of collagen peptides, vitamin C, Omega-3 fatty acids, and copper can be used as daily supplements. Different types of topicals and supplements are used to improve different aspects of skin conditions based on different working mechanisms. For example, as shown in Table 2, topical vitamin C can improve collagen synthesis and provide strength and structure to the skin, and topical peptides can strengthen the skin barrier, increase water retention, and stimulate collagen production, thereby improving the appearance of wrinkles in aging skin, while copper supplement can stimulate fibroblasts and collagen production and elastin fiber components, crosslink collagen, promote collagen fibril formation, and protect against oxidative damage.

The forms in the topical and/or supplement blend can be modified according to the individual's other medications (e.g., genetic statuses as described below) when a customized skin regimen is given.

### Sebum Biomarkers

In one aspect, provided herein are methods of determining a level of skin biomarkers from sebum of a subject (i.e., sebum biomarkers).

In one aspect, provided herein are methods of providing a customized skin regimen based at least on the sebum biomarkers of the subject, the method comprising: obtaining or having obtained levels of a plurality of skin biomarkers from one or more samples obtained from the subject, and determining the customized skin regimen for the subject using at least the plurality of skin biomarkers. The skin biomarkers comprise the sebum biomarkers, including one or more lipids obtained from sebum of the subject.

Sebum is the oily and waxy substance secreted by the sebaceous glands of the skin. It is a lipid-rich matrix, consisting of glycerolipids, free fatty acids, cholesterol, cholesterol esters, squalene, and wax esters. The component percentages of sebum are usually 30-50% glycerolipids, 15-30% free fatty acids, 1.5-2.5% cholesterol, 3-6% cholesterol esters, 12-20% squalene, and 26-30% wax esters. Alterations in the relative abundances of these sebum components are reflected by sebum biomarkers, which provide information about cutaneous diseases. Cutaneous diseases are conditions affecting the skin such as rashes, inflammation, itchiness, or other skin changes.

Acne is a common concern and sebum is one of the main causative factors affecting acne occurrence. Increased sebum secretion can induce acne occurrence, and increasing evidence indicates sebum component changes are also strongly related to susceptibility to acne.

The measurement of sebum biomarkers is obtained using an absorbent medium or material. This measurement may help with quantifying the lipid concentration in a person's sebum, thereby understanding the cause and severity of skin ailments (e.g., acne).

Sebum biomarkers may also reflect the internal metabolic state of the body, such as hormonal levels, oxidative stress, or inflammation, that relates to skin health.

### Urology Biomarkers

Urology biomarkers (e.g., proteins/peptides) are substances that can be measured in urine to indicate the presence or progression of a urological condition. Urology biomarkers may help with skin health diagnosis, prognosis, and treatment. Skin urological biomarkers are currently being studied in clinical practice, which are used in combination with other tests and clinical findings to make a diagnosis or prognosis of skin health. In some embodiments, the urine analysis for UV damage metabolites and oxidative stress loads can be conducted in the methods disclosed herein.

In some embodiments, the methods comprise obtaining or having obtained urology biomarkers from urine of the subject.

In some embodiments, the urology biomarkers of the subject comprise one or more of UV damage metabolites and oxidative stress loads from the urine of the subject.

### Genetic Testing

In some aspects, provided herein are methods for determining a customized skin regimen for a subject, the method comprising: obtaining or having obtained levels of a plurality of skin biomarkers from one or more samples obtained from the subject; obtaining or having genetic statuses of one or more genomic locations of a plurality of genes from the one or more samples obtained from the subject; and determining the customized skin regimen for the subject using both the levels of the plurality of skin biomarkers and the genetic statuses of one or more genomic locations of a plurality of genes.

In some aspects, obtaining or having obtained genetic statuses of one or more genomic locations is based on determining deficiency predispositions at the genetic level (e.g., single nucleotide polymorphisms (SNPs), or other genetic mutations). Genetic testing for predispositions can assist in providing information about a subject's skin health. Table 4 provides exemplary skin-related polymorphisms that can be assessed and the polygenic risk associated with the polymorphisms.

Since genetic polymorphisms influence genes that are involved in melanocyte pigmentation, skin tanning response, hydration, collagen and elastin synthesis, and skin inflammation, assessing genetic polymorphisms is an important determinant of phenotypic variation and skin disease susceptibility. There are various processes governed by specific genes that can affect skin statuses. Alterations in these genes may affect their normal functioning which can affect the skin statuses. These alterations can be assessed through genetic testing. For example, polymorphisms in the gene encoding may relate to water loss from the skin that gives rise to reduced skin moisturization capacity. Alterations in the BCMO1 gene may show a vitamin A deficiency that accelerates the presence of wrinkles and ultimately skin aging. Genetic testing can be carried out using any appropriate method known in the art, including but not limited to, reverse transcription polymerase chain reaction (RT-PCR) or quantitative-PCR (qPCR).

In some embodiments, the methods comprise obtaining or having obtained genetic information of the subject, the genetic information relating to melanin synthesis, moisturization, skin inflammation, melanocyte damage, collagen degradation, elastin synthesis, antioxidant, and nutritional needs.

In some embodiments, the genetic information of the subject comprises genetic statuses of one or more genomic locations of a plurality of genes.

In some embodiments, the genetic statuses comprise presence or absence of a polymorphism. In some embodiments, the genetic statuses comprise presence or absence of a polymorphism in a gene. In some embodiments, the genetic statuses comprise presence or absence of one or more polymorphisms. In some embodiments, the genetic statuses comprise presence or absence of one or more polymorphisms in one or more genes. In some embodiments, the genetic statuses comprise presence or absence of two, three, four, five, six, seven, eight, nine, ten, or more polymorphisms. In some embodiments, the one or more polymorphisms and/or genes are selected from Table 4.

In some embodiments, the polymorphism is one or more of rs1426654, rs16891982, rs1042602, rs1393350, rs6058017, rs74653330, rs1393350, rs1805008, rs1015362, rs4911414, rs12203592, rs1805007, rs1805009, rs17553719, rs61816761, rs138726443, rs150597413, rs20541, rs2201841, rs1801133, rs610604, rs17728338, rs1800795, rs4646994, rs1169732, rs1799750, rs7787362, rs1001179, rs1050450, rs1800566, rs4880, rs2234694, rs7501331, rs12934922, rs4654748, rs602662, rs33972313, rs2282679, rs1801131, rs1801133, or rs174547.

In some embodiments, the plurality of genes comprises one or more of SLC24A5, SLC45A2, TYR, ASIP, OCA2, MC1R, IRF4, AQP3, FLG, IL-13, IL23R, MTHFR, TNFAIP3, TNIP1, IL-6, ACE, MMP9, MMP1, ELN1, CAT, GPX1, NOQI, SOD2, BCMO1, NBPF3, FUT2, SLC23A1, GC, MTHFR, or FADS1.

### Polygenic Risk Factor

The effect of skin-related genetic predispositions can also be considered and addressed in the methods disclosed herein. The effect of exemplary alleles for the respective exemplary skin-related genetic polymorphism provided in Table 4 can be determined by calculating a genotype value for each subject. The genotype value can be determined based on the strength of each exemplary SNP that affects the nutrient levels in a subject. For example, risk genotypes can be assigned a higher or a lower score depending on the risk associated with a given SNP based on the effect of the SNP. For example, SNPs that are known to have more pronounced deleterious effects can be given a higher score.

In some aspects, for each SNP category, the cumulative effect of its SNPs can be calculated, resulting in a “Polygenic risk factor” (PRF). Exemplary PRFs for a given SNP category can be multiplied in the dosage formula to determine the dosage for the respective SNP category. Table 4 includes a summary of the genotype values and the PRFs for the respective exemplary skin-related SNPs.

A PRF is an estimate of an individual's genetic risk for some trait, obtained by aggregating and quantifying the effect of many common variants in the genome. The PRF provides a measure of the risk of a disease or disease-related trait for an individual due to the individual's genes across the human genome. In some embodiments, a PRF can be an average of the polymorphisms in a SNP category. For example, when 13 polymorphisms are used in melanin synthesis in Table 4, the PRF is the average of the 13 polymorphisms.

In some embodiments, the PRF can be used to provide ‘Topicals Suggestions’ based on the risk associated with the conditions caused by skin-related SNPs (see Table 5). In some embodiments, the PRF can also be used to provide ‘Supplement Suggestions’ based on the risk associated with the conditions caused by skin-related SNPs (see Table 6).

In one embodiment, the skin questionnaire may determine the supporting topicals and/or supplements required for certain conditions, and the PRF may determine the supporting topicals and/or supplements required for conditions brought about by skin-related SNPs. In one embodiment, the PRF further determines the amount of recommended supporting topicals and/or supplements required for conditions brought about by skin-related SNPs (tables 4 and 5).

In one embodiment, the topical and/or supplement blend can be modified according to the individual's genetics.

In some embodiments, the methods further comprise determining a polygenic risk factor according to the genetic statuses of the one or more locations of the plurality of genes.

In some embodiments, determining the personalized dosage further comprises determining the personalized dosage of the skin topicals and/or supplements for the subject according to the polygenic risk score.

In some embodiments, determining a polygenic risk factor according to the genetic statuses of the one or more locations of the plurality of genes comprises: for each of the one or more locations of a gene, assigning a genotype value according to presence or absence of a polymorphism at the location of the gene; and combining the genotype values across the locations of the plurality of genes.

In some embodiments, the one or more polymorphisms are selected from Table 4. For example, a risk factor can comprise testing for one, two, three, four, five, or more of the polymorphisms provided in Table 4.

### Topical Suggestions for SNPs

The skin product can be customized to meet an individual's skin needs based on his/her genetic skin profile. In some aspects, genetic testing can aid in determining the best form of topicals and supplements based on polymorphisms affecting biological pathways that impact skin health. Table 5 lists topical suggestions for various SNP categories including melanin synthesis, moisturization, skin inflammation, melanocyte damage, collagen degradation, elastin synthesis, antioxidant status, vitamin A deficiency, vitamin B6 deficiency, vitamin B12 deficiency, vitamin C deficiency, vitamin D deficiency, folate deficiency, and omega fatty acid deficiency.

As mentioned above in Table 4, genetic polymorphisms influence genes, and PRFs quantify such influences. In some embodiments, the topical suggestions in Table 5 are determined based on the PRFs. For example, several vitamin A polymorphisms in the BCMO1 gene may result in low levels of vitamin A in the body thus giving rise to a vitamin A deficiency. This may cause wrinkles and accelerate aging. In such exemplary cases, topicals such as retinol, vitamin E, resveratrol, baicalin, B vitamins, and vitamin C are recommended to be applied on a particular area of the skin, to improve the skin health issues caused by vitamin A deficiency.

### Supplement Suggestions for SNPs

Supplements can help with improving various factors affecting the skin. The results from the genetic analysis help ascertain the fundamentals that are affecting a person's skin. Based on the genetic analysis including the PRFs in Table 4, appropriate supplements can be suggested to improve the root cause of the underlying skin conditions. The phenotypic outcomes of the tested SNPs along with the appropriate set of supplements that help improve the skin conditions are shown in Table 6. This table lists supplement suggestions for various SNP categories including melanin synthesis, moisturization, skin inflammation, melanocyte damage, collagen degradation, elastin synthesis, antioxidant, and nutritional needs.

Continuing with the exemplary case where several vitamin A polymorphisms in the BCMO1 gene are associated with vitamin A deficiency, leading to wrinkles and accelerating aging, supplements may also be administrated to the individual in addition to the application of topical medications. Subjects may be supplemented with the active form of vitamin A, epigallocatechin gallate, collagen, nicotinamide riboside, nicotinamide mononucleotide, coenzyme Q10, vitamin C, or curcumin, which is readily used by the body, to optimize vitamin A levels. In some embodiments, the general supplements are selected from Table 6. The supplements are determined based on the PRFs calculated in Table 4. While the concentration or the degree to which each supplement is added to the customized skin product may change when there is a change of PRFs derived from the genetic testing, the concentration or degree of the supplement can only change up to advisable values.

### Epigenetics

In some aspects, obtaining or having obtained genetic statuses of one or more genomic locations is based on determining methylation patterns of certain genes that are believed to affect the human skin and aging from different aspects.

Methylation is a biochemical process of adding a methyl group (CH₃) to a molecule, which can influence gene expression, protein function, and cellular behavior. In the context of skin health and beauty, methylation plays a role in aging, skin cell regeneration, and even in the skin's ability to repair damage from environmental stressors, such as UV rays, pollution, and inflammation. Methylation impacts DNA and gene regulation, including those genes involved in the aging process. When people gets older, methylation patterns in skin cells can change, potentially leading to less effective skin repair and renewal. In some cases, “aberrant” methylation can switch off genes that promote youthful skin structure (e.g., collagen production), leading to wrinkles, sagging, and dullness. In addition, DNA damage in skin cells is caused by factors like UV radiation and oxidative stress. Proper methylation helps regulate DNA repair processes. Further, methylation is important in detoxification pathways. In the liver, methylation helps detoxify harmful compounds, but it's also relevant for skin cells, which are constantly exposed to toxins from the environment. Even more, epigenetics described above is also highly connected to methylation. For these reasons, personalized skincare products might be able to target individual epigenetic and/or methylation profiles, tailoring treatments that specifically address how a person's skin ages or reacts to the environment.

Methods for generating epigenetics information can involve obtaining a sample from a subject, processing the sample to obtain nucleic acids (e.g., cell free DNA), and performing a methylation assay (e.g., assay 120). A methylation assay can, in various embodiments, include a performing a conversion (e.g., a bisulfite conversion) of nucleic acids, amplifying certain target regions of the converted nucleic acids, and sequencing nucleic acids to generate the epigenetics information. Further details for performing methylation assays (e.g., nucleic acid conversion, amplification, sequencing) to generate epigenetics information are described in WO2020069350, which is incorporated by reference in its entirety.

In various embodiments, performing the methylation assay results in generation of epigenetics information. Epigenetics information includes methylation statuses for a plurality of genomic sites. The plurality of genomic sites may be one or more CpG sites whose differential methylation are informative for the skin condition of a subject. A CpG site is portion of a genome that has cytosine and guanine separated by only one phosphate group and is often denoted as “5′-C-phosphate-G-3′”, or “CpG” for short.

In various embodiments, performing an assay (e.g., assay 120) involves processing nucleic acids (e.g., cfDNA fragments) from a sample (e.g., liquid biopsy sample). In various embodiments, processing nucleic acids includes treating the nucleic acids to capture methylation modifications. In various embodiments, processing nucleic acids to capture methylation modifications includes performing deamination of cytosine residues. Other techniques include but are not limited to enzymatic methods. In various embodiments, processing nucleic acids to capture methylation modifications includes performing any of nucleic acid amplification, polymerase chain reaction (PCR), methylation specific PCR, bisulfite pyrosequencing, single-strand conformation polymorphism (SSCP) analysis, methylation-sensitive single-strand conformation analysis restriction analysis, high resolution melting analysis, methylation-sensitive single-nucleotide primer extension, restriction analysis, microarray technology, next generation methylation sequencing, nanopore sequencing, and combinations thereof.

In various embodiments, performing deamination of cytosine residues is useful for determining methylation statuses of nucleic acids from a sample. Performing deamination involves providing or exposing nucleic acids from a sample to a deaminating agent. In various embodiments, performing deamination of cytosine residues involves performing selective deamination. Selective deamination refers to a process in which cytosine residues are selectively deaminated over 5-methylcytosine residues. Deamination of cytosine forms uracil, effectively inducing a C to T point mutation to allow for detection of methylated cytosines. Methods of deaminating cytosine are known in the art, and include bisulfite conversion and enzymatic conversion. Bisulfite conversion enables highly efficient conversion of unmethylated cytosines to uracils of DNA from samples such as whole blood or plasma, cultured cells, tissue samples, genomic DNA, and formalin-fixed, paraffin-embedded (FFPE) tissues. Bisulfite conversion can be performed using commercially available technologies, such as Zymo Gold available from Zymo Research (Irvine, CA) or EpiTect Fast available from Qiagen (Germantown, MD). In certain embodiments, the enzymatic conversion comprises subjecting the nucleic acid to TET2, which oxidizes methylated cytosines, thereby protecting them, and subsequent exposure to APOBEC, which converts unprotected (unmethylated) cytosines to uracils.

The method disclosed herein identify methylation patterns for certain genes that are believed to affect human skin from different aspects. The exemplary genes and the associated factors for skincare are listed in Table 7, which includes seven categorized genes, their relevant function to skin and their respective methylation-related effects. In one example, gene ELN relates to elastin production and is categorized into the ‘skin firming and elasticity” category. Methylation of gene ELN reduces elastin production, causing skin to lose firmness and elasticity. Accordingly, by testing the methylation of this gene, its allows to find ingredients or nutrients that support methylation pathways.

In some embodiments, the testing of methylation patterns of specific genes includes analyzing DNA to determine the extent and pattern of methylation at specific sites within the genome. This includes analyzing DNA from blood, saliva, buccal swabs, skin biopsy, urine samples, and the like using bisulfite conversion, sequencing, or methylation-specific PCR, pyrosequencing, among other possible methods.

In some embodiments, a methylation score is further generated based on the tested methylation patterns of certain genes. In one example, based on the testing of methylation patterns in these genes, the biological age (also referred to ‘methylation age’) can be determined, which measures how old an individual's body appear on a molecular level, often linked to lifestyle, stress, and disease risk. In one example, the methylation age is calculated based on the Equation (1):

Methylation Age in each category=Σ(Methylation Level of Gene×Weight) (1)

Here, the category refers to one of the seven categories shown in Table 7, and weight for each gene can be also found in Table 7.

In some embodiments, an epigenetics age is further generated based on the determined methylation age and a determined phenotype age as will be described later. In one example, the epigenetics age is calculated based on the following Equation (2):

Epigenetics Age=Average (Methylation Age+Phenotype Age) (2)

In some embodiments, determining the customized skin regimen for a subject using both the levels of the plurality of skin biomarkers and the genetic statuses of one or more genomic locations of a plurality of genes includes determining the customized skin regimen for the subject using the determined methylation score, among other possible scores or parameters. In one example, the customized skin regimen for the subject is determined based on the determined methylation age in each of the aforementioned seven categories shown in Table 7. In another example, the customized skin regimen for the subject is determined based on the determined epigenetics age in each category.

### Skin Glycation

The non-enzymatic reaction between reducing sugars (including fructose and glucose) and proteins, lipids, or nucleic acids leads to the formation of advanced glycation end products (AGEs). These products are complex heterogeneous molecules causing browning and fluorescence. They are ubiquitous in human and accumulate widely in tissues with normal aging. They trigger inflammatory pathways by forming crosslinks, altering protein structure, or binding to AGE receptors. AGEs can be endogenously produced via the Maillard reaction or exogenously ingested (e.g., through food consumption and tobacco). The increased production of AGEs may lead to accelerated aging. The AGEs, carboxyethyl-lysine (CEL), [N-(carboxymethyl)lysine](CML), glucosepane, and pentosidine have been associated with skin aging. Their binding with skin collagen increases with increasing age.

Skin glycation is defined as the aging reaction of naturally occurring sugars and dermal proteins. In the skin, collagen is more likely to be glycated due to a slow turnover rate. AGEs act directly on skin cells, by decreasing cell functions via the activation of inflammatory signaling pathways and oxidative stress through cell surface receptors, resulting in skin problems such as dullness, pigmentation, and wrinkles. Moreover, AGEs crosslink with collagen and elastin in the extracellular matrix and promote the loss of skin elasticity. UV radiation also leads to excessive AGEs formation. The UV radiation combined with AGEs action then leads to accelerated skin aging.

The methods disclosed herein test the urine and blood of an individual for the presence of important AGEs associated with skin glycation. The most important AGEs to be tested are listed in Table 8, which include carboxymethyl lysine (CML), carboxyethyl lysine (CEL), glucosepane, and pentosidine, as applicable. This test can help estimate the degree of skin glycation. For example, if pentosidine in skin collagen is presented in an individual's urine sample, this individual's food and routine will be adjusted to slow down skin aging and related disorders (e.g., wrinkles, age spots, etc). In some embodiments, the test of skin glycation can be used to generate appropriate dietary, supplementation, and lifestyle recommendations.

In some embodiments, the methods further comprise obtaining or having obtained levels of one or more advanced glycation end products from urine or blood of a subject.

In some embodiments, the advanced glycation end products comprise one or more of carboxymethyl lysine (CEL), carboxymethyl lysine (CML), glucosepane, or pentosidine.

### Skin Microbiome Assessment

The skin is the largest organ with an abundance of folds, invaginations, and specialized niches that host a wide range of microorganisms. The skin microflora comprises bacteria, fungi, and viruses. Commensal microorganisms occupy a wide range of skin niches and protect the skin against invasion by more pathogenic or harmful organisms, which contribute to tissue integrity and immune homeostasis. The bacterial genera Streptococcus, Corynebacterium, Propionibacterium Brevibacterium, and Micrococcus are the major skin colonizers. Malassezia spp., are the fungal species that are especially prevalent in sebaceous areas. The skin commensal virome comprises eukaryotic viruses and bacteriophages, where bacteriophages are predominant.

Many common skin conditions are considered to have an underlying microbial contribution. The commensal skin bacterium, P. acnes is associated with acne. Acne is an inflammatory disorder of the pilosebaceous unit (hair follicle, hair shaft, and sebaceous gland). An increase in P. acnes may cause the elevated secretion of lipases, proteases, and hyaluronidases that injure the tissue lining of the pilosebaceous unit, eventually resulting in acne. Atopic dermatitis (AD), which is commonly known as eczema or contact dermatitis, is characterized by inflammation, redness, and irritation of the skin. AD is associated with S. aureus; however, the pathogenesis is to be elucidated. Treatment with antibiotics can help improve the conditions associated with these pathobionts. Additionally, from the skin aging perspective, there has been a reduction in the relative abundance of the dominant skin genus Cutibacterium in the cheek and forehead while there was an increased proportion of Corynebacterium in the same regions of the face. With respect to phenotypic features, there was an increased abundance of Corynebacterium, Neisseriaceae, Chryseobacterium, Prevotella, and Veillonella in older skin. This is also associated with wrinkles and skin pigmentation.

As the skin microbiome plays an important role in skin conditions and skin aging, assessing these micro-organisms for their relative abundances on the skin acts as an effective tool in getting a good understanding of skin health. This also enables the employment of dietary and supplementation interventions, which improve the commensal abundance and hence improve skin health.

Table 9 provides an exemplary illustration of skin microbiome assessment. This assessment includes evaluation of the level of skin microbiota on particular skin surfaces of an individual leading to various skin conditions or ailments. The skin conditions include acne vulgaris, rosacea, atopic dermatitis, seborrheic dermatitis, pigmented skin, increased sebum, wrinkles, skin aging, etc.

In some embodiments, the method comprises obtaining or having obtained abundance of one or more microorganisms from skin niches of a subject.

In some embodiments, the microorganisms comprise one or more of Propionibacterium acnes, Propionibacterium avidum, or Propionibacterium granulosum, optionally from forehead of the subject.

In some embodiments, the microorganisms comprise Propionibacterium acnes, optionally from cheek of the subject.

In some embodiments, the microorganisms comprise one or more of Staphylococcus epidermidis, Corynebacterium spp, or Propionibacterium acnes, optionally from nasolabial fold of the subject.

In some embodiments, the microorganisms comprise Propionibacterium acnes, optionally from chin of the subject.

In some embodiments, the microorganisms comprise Propionibacterium acnes, optionally from chin of the subject.

In some embodiments, the microorganisms comprise one or more of Corynebacterium kroppenstedtiin or Staphylococcus epidermidis, optionally from cheek of the subject.

In some embodiments, the microorganisms comprise Corynebacterium kroppenstedtiin, optionally from nasolabial fold of the subject.

In some embodiments, the microorganisms comprise Staphylococcus epidermidis, optionally from eyelid of the subject.

In some embodiments, the microorganisms comprise Staphylococcus epidermidis, optionally from cheek of the subject.

In some embodiments, the microorganisms comprise Corynebacterium species, optionally from chin of the subject.

In some embodiments, the microorganisms comprise Malassezia spp, optionally from cheek of the subject.

In some embodiments, the microorganisms comprise one or more Eikenella corrodens, Cutibacterium, Micrococcus luteus, or Kocuria, optionally from cheek of the subject.

In some embodiments, the microorganisms comprise Acinetobacter, optionally from cheek of the subject.

In some embodiments, the microorganisms comprise Corynebacterium, optionally from cheek of the subject.

In some embodiments, the microorganisms comprise one or more of Corynebacterium amycolatum, Proteobacteria, Firmicutes, Bacteroidetes, or Propionibacterium, optionally from forehead of the subject.

In some embodiments, the microorganisms comprise Propionibacterium, optionally from cheek of the subject.

In some embodiments, the microorganisms comprise one or more of Corynebacterium, Lactobacillus, or Cutibacterium, optionally from face of the subject.

### Demographics

The effect of demographics can also be considered and addressed in the methods disclosed herein. Demographics can be a component used in providing a customized skin regimen. Changes in demographics such as age and gender can alter a subject's skin conditions. Thus, demographics can be factored into the evaluation of the skin regimen, for determining topicals applied to a particular area of the skin, for determining supplementing nutrients, and for monitoring the progress of the implemented topicals and dietary/supplement interventions in a subject.

An individual's age is a critical factor affecting skin health. Advancing age is associated with structural and functional changes in extracellular matrix components such as collagens, elastin, and proteoglycans that are required to provide tensile strength, elasticity, and hydration to the skin. The accompanied phenotypic changes are loss of plump and smooth skin, visible veins and bone structure, and increased scarring.

Gender is another key factor influencing skin health. An individual's skin is influenced by sex-related differences in anatomy, physiology, epidemiology, and the manifestations of several diseases. Skin parameters such as hydration, transepidermal water loss, sebum, microcirculation, pigmentation, and thickness are generally higher in men while skin pH is higher in women. These differences may be related to hormones. For example, sebum content can be elevated in men because sebum is highly influenced by sex hormones.

These sex-related differences may also play a role in disease susceptibility with infectious diseases being presented more in men but psychosomatic problems, pigmentary disorders, certain autoimmune and allergic diseases being more prevalent among women. However, the mechanisms that underlie sex-related differences in skin diseases are yet to be elucidated.

In some embodiments, the methods disclosed herein further comprise obtaining or having obtained demographics of the subject, and determining the customized skin regimen for the subject based on the demographics of the subject.

In some embodiments, the demographics comprise one or more of an age or gender of the subject.

### Phenotypic Assessment

The phenotypic appearance of the skin is an obvious indicator of skin health, which is another factor considered and addressed in the methods disclosed herein. A visual evaluation may provide the status of the skin and also represent tangible improvements expected by individuals. Such visual evaluations help with addressing visible skin concerns, and have widely been used in diagnosis as well as experimental dermatology. However, a crucial, visual assessment can be subjective and differs from one assessor to the other. To overcome this drawback, a digital system is proposed herein in this disclosure to analyze the phenotypic features of the skin.

In some embodiments, the system disclosed herein may involve capturing images of an individual's skin. The captured image can be of the individual's skin from any anatomical location of the individual. In various embodiments, the captured image is of the individual's face. These images may be taken at different angles. For example, the images can be taken from the front of the face and from two sides of the face. Using the images, the inbuilt system may grade the severity of each phenotypic parameter. A consensus on the degree of severity may be determined with the help of experts in the field. In some embodiments, the system disclosed herein may provide a questionnaire to an individual in the phenotypic appearance assessment, as described later in the ‘Skin Questionnaire’ section. Based on the feedback from the questionnaire, the inbuilt system may grade the severity of each phenotypic parameter.

Table 10 illustrates an exemplary grading system for grading the severity of phenotypic parameters from seven different categories, namely, an anti-aging and wrinkle reduction category, a lymphatic drainage category, a skin firming and elasticity category, a brightening and pigmentation category, a UV/oxidative damage/DNA/cell damage category, an anti-inflammatory and soothing category, and a hydration and skin barrier repair category. As illustrated in the figure, the inbuilt grading system includes a picture-based grading algorithm and a questionnaire-based algorithm. The picture-based grading algorithm provides a scale of 1-10 (where 1 is the best and 10 is the worst) for each of the seven categories based on the image evaluation from different aspects. The questionnaire-based algorithm provides a score for each category based on the information collected form the questionnaire. As can be seen in Table 10, instead of the scale of 1-10 for all seven categories, the questionnaire-based algorithm provides different scales for different categories, for example, a scale of 0-3 for ‘lymphatic drainage’, a scale of 0-6 for ‘anti-aging and wrinkle reduction’, and a scale of 0-8 for ‘hydration and skin barrier repair”.

In some embodiments, the inbuilt system disclosed herein further generates a phenotype age for each of the seven categories. For example, based on the obtained points from the picture-based grading and questionnaire-based grading (e.g., the sum of the two grading outcomes), a phenotype age is further determined for the subject under the assessment for each of the seven categories. Table 11 illustrates an exemplary grading principle for determining the phenotype age of a subject under the assessment. In the exemplary grading illustrated in the table, the phenotype age of the subject under the assessment can be determined to have a phenotype age of 30-70. However, it should be noted that the phenotype ages can have other different ranges, which is not limited in the disclosure. In some embodiments, the determined phenotype ages for different categories may be different, which can provide information which category(ies) should be focused when determining the customized topical, supplements and lifestyle suggestions. For example, if ‘brightening and pigmentation’ has a determined phenotype age of 40 while all other six categories have a determined phenotype of 30, the determined customized topical, supplements and lifestyle suggestions are more focused on the skin brightening and pigmentation. In some embodiments, based on the obtained phenotype age for each of the seven categories, the disclosed methods further determine the epigenetics age, as described earlier.

Table 12 shows exemplary suggestions for topical, supplements, and lifestyles, which are generated based on phenotypic assessment over a set of phenotypic parameters such as wrinkles, periorbital-around eyes, etc. The suggestions may be determined based on the graded severity of each phenotypic parameter. For example, when determining topical supplementation, a higher severity may cause the system to provide a higher concentration of topical ingredients. It should be noted, however, that an increase in topical ingredients will only take place up to one or more safe, advisable limits.

In some embodiments, the methods disclosed herein further comprise obtaining or having obtained an image of a face from a subject, grading severities of one or more phenotypic parameters for the face of the subject based on the image, and determining the customized skin regimen for the subject based on the severity of the one or more phenotypic parameters. In some embodiments, the grading is point-based and have a scale of 1-10 where 1 represents the best and 10 represents the worst.

In some embodiments, the methods disclosed herein further comprising obtaining or having obtaining feedback for a questionnaire sent to a subject, grading severities of one or more phenotypic parameters for the subject based on the feedback of the questionnaire, and determining the customized skin regimen for the subject based on the severity of the one or more phenotypic parameters.

In some embodiments, the methods disclosed herein further comprising obtaining or having obtaining a phenotype score for one or more categories for a subject and determining the customized skin regimen for the subject based on the phenotype score of the one or more categories. In one example, the aforementioned phenotype age may be used as the phenotype score for each of the seven categories described above.

In some embodiments, the phenotypic parameters comprise one or more of wrinkles, periorbital-around eyes (i.e., puffy eyes), nasolabial folds (i.e., laugh lines), worry lines, uneven skin tone or pigmentation, or age spots (i.e., liver spots). In some embodiments, the phenotypic parameters are categorized into seven different categories as shown in Table 10 and Table 11.

### Skin Questionnaire

Individuals have various concerns as well as goals with respect to their skin. Dermatology studies state that patients' participation in medical decision-making has been associated with higher satisfaction with their care, which results in higher compliance to skin regimens and better skin health outcomes. Therefore, individuals' concerns must be taken into consideration, and their needs must be met while creating customized products for individuals.

In some aspects, a skin questionnaire is employed to query an individual's concerns and needs. The questionnaire addresses various factors such as skin goals, type of skin, sun exposure, and skin proneness to external factors, sensitivity to skin products, etc. It also takes into account whether individuals are undergoing or have undergone skin treatments or cosmetic procedures because certain treatments and cosmetic procedures may alter the skin's structure and composition, which can in turn affect the skin's ability to absorb and react to skincare products.

The skin questionnaire can assist with the selection of suitable skin topical and/or supplement forms to ameliorate certain symptoms and overall improve the subject's skin health. The skin questionnaire may comprise the following questions: if any of the following goals was a primary goal for the subject's skin: UV protection, anti-wrinkling, anti-pigmentation, moisturization, radiant complexion; if the subject's skin was dry; if dry, was the skin mild dry, very dry, or extremely dry; if the subject had specific oily areas on his/her face while the rest of the areas may experience a certain level of dryness; if the subject's skin was prone to acne; the amount of time (1 hour, 1-2 hours, or >2 hours) the subject got sun exposure every day; if the subject has ever or is currently receiving skin treatments; if receiving or has received skin treatments, list the treatments; if the subject has stinging or burning after applying any/most skin products; if the subject has excessive scratching, excessive peeling, pus and oozing; if the subject has excessive scratching, excessive peeling, pus and oozing, then see a dermatologist; any external factors affecting the subject's skin including sun and humidity, wind and cold, or others; if the subject has a proper sleep cycle.

The questionnaire can help determine the presence of complications or symptoms associated with altered topical and/or supplement levels, susceptibility to side effects that may be associated with topicals and/or supplements, presence of comorbidities and their symptoms, or ongoing medication usage by the subject. The questionnaire can assess these aspects using direct questions which could be answered with either a ‘yes’ or a ‘no.’ The questionnaire can also receive the subject's selections for certain other answers to the questions.

In some embodiments, a grading system can be further applied to generate a questionnaire score for the skin questionnaire. Points can be assigned to each selected answer, as illustrated in Table 13. For example, if the question is ‘How often do you notice fine lines or wrinkles on your skin,’ the answer can be ‘Never,’ ‘Occasionally,’ ‘Frequently,’ and ‘Always.’ The four different answers have respective points of 1, 2, 3, and 4. For other questions included in the questionnaire, each answer also has a corresponding point assigned in advance. Based on the answers feedback from the questionnaire, one or more questionnaire scores can be then generated. For example, if the questions are categorized into different categories, a questionnaire score may be obtained for each categories (e.g., sum of the points corresponding to the selected answers to all questions included in the category). In some embodiments, an overall questionnaire score can be generated based on the selected answers to all questions included in the questionnaire.

Responses to the skin questionnaire or the determined questionnaire score(s) can determine appropriate suggestions for topicals, supplements, and lifestyle of the subject. Table 14 provides exemplary responses for the skin questionnaire and the customized topical, supplement, and lifestyle recommendations.

For example, if an individual aims to seal the moisture of his/her skin, different forms of topicals are available to help the individual achieve his/her goal. Hyaluronic acid can act as a barrier to prevent water loss through evaporation, reduces trans epidermal water loss (TEWL), and improves the skin's ability to retain moisture. Similarly, to prevent dryness and dehydration, glycerin can be applied to create a thin, occlusive layer to help seal in moisture and prevent water loss through evaporation, shea butter resembles the natural lipids found in the skin to enhance moisture retention, while jojoba oil resembles the natural sebum to form a non-greasy protective barrier on the skin's surface to prevent trans epidermal water. In the meantime, the individual is also recommended to intake supplements such as hyaluronan, ceramides, and collagen to incorporate with the above-mentioned skin barriers to strengthen the skin and improve moisture retention. Lastly, if the individual is taking long hot baths, he/she may want to take quicker shower with colder water. If the individual is not using a humidifier, he/she may try to use it. This kind of lifestyle suggestions as well as the use of topicals and/or supplements can be personalized and used by specific persons to meet their specific skin needs.

In general, the combination of skin biomarker testing, demographic considerations, and the skin questionnaire can enable improving the individual's overall skin health.

In some embodiments, the methods disclosed herein further comprise obtaining or having obtained one or more responses to a skin questionnaire from the subject and determining the customized skin regimen for the subject based on the one or more responses from the subject. The customized skin regimen includes one or more suggestions for topicals, supplements, or lifestyles determined based on the one or more responses, and the one or more suggestions can be selected from Table 14.

In some embodiments, the skin questionnaire comprises one or more questions related to skin goals, type of skin, sun exposure, or skin proneness to external factors.

### Additional Tests

The method disclosed herein offers testing for various skin parameters using biomarkers (serum, sebum, urine, any biospecimen) along with many genetic predispositions that may affect an individual's skin health. These results are used to create customized skin products that would improve the individual's skin health based on his/her skin profile. However, the skin is also affected by other factors such as metabolism and nutrition. Therefore the methods disclosed herein also offer tests to assess metabolism and nutrient levels to help understand the other fundamental causes affecting the skin.

### HormonePro

Hormones are crucial for skin health as they play an important role in the development and physiological function of human skin tissues. The skin has been the target for various hormones in the body. For example, sebaceous glands are the targets for androgen steroids that are secreted by the gonads and the adrenal cortex. Some hormones are even produced in the skin. For example, the circulating androgens dehydroepiandrosterone (DHEA) and androstenedione are converted to testosterone or 5α-dihydrotestosterone (5α-DHT) in the skin. This makes the skin responsible for considerable amounts of the circulating 5α-DHT levels. The skin's cell surface expresses receptors for peptide hormones while the receptors for steroid and thyroid hormones are found in the cytoplasm or nuclear compartments of skin cells. From the modern dermato-endocrinologic point of view, the skin is not only the recipient but also the orchestrator of various endocrine signals.

Consequentially, assessing hormone levels is critical for skin health. In some aspects, the methods disclosed herein further comprise a ‘HormonePro’ test which tests for various urine and blood hormones along with genetic assessment for SNPs affecting hormone functions. HormonePro tests could provide insights into a subject's hormone profile that is correlated to the status of the subject's skin. Based on HormonePro test results, a customized skin regimen (including dietary and supplement suggestions) can be generated to improve hormone functions.

Table 15 provides an exemplary list of biomarkers that can be tested in the ‘HormonePro’ test along with the respective directionality, exemplary supplements, and dosages for males that can improve the indicated biomarker levels and the associated skin conditions.

In some embodiments, the methods disclosed herein comprise obtaining or having obtained the levels of one or more hormones (i.e., hormone biomarkers). Obtaining the levels of hormones comprises determining one or more combinations of levels of the one or more hormones. The hormone markers are obtained from urine samples of a subject.

In some embodiments, the subject is a male (Table 15), and the hormone markers comprise one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG).

When the subject is a male, in some embodiments, determining one or more combinations of levels of the one or more hormones comprises determining one or more of Estriol (E3)/(E1+E2), 2-OH (E1+E2)/16a-OH E1, 2-MeO E1/2-OH E1, 4-MeO E1/4-OH E1, 4-MeO E2/4-OH E2, testosterone (T)/Epi-T, b-pregnanediol/E2, cortisol/cortisone, or metabolized cortisol (THF+THE), or Prostaglandin (Pg)/E2.

Similar to Table 11, Table 15 provides an exemplary list of biomarkers that can be tested in the ‘HormonePro’ test along with the respective directionality, exemplary supplements, but includes dosages for females.

In some embodiments, the methods disclosed herein further comprise obtaining or having obtained the levels of one or more hormones (hormone biomarkers). Obtaining the levels of hormones comprises determining one or more combinations of levels of the one or more hormones. The hormone markers are obtained from urine samples of a subject.

In some embodiments, the subject is a female (table 11), the hormones comprise one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5α-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG).

When the subject is a female, in some embodiments, determining one or more combinations of levels of the one or more hormones further comprises determining one or more of E3/(E1+E2), 2-OH (E1+E2)/16a-OH E1, 2-MeO E1/2-OH E1, 4-MeO E1/4-OH E1, 4-MeO E2/4-OH E2, T/Epi-T, b-pregnanediol/E2, cortisol/cortisone, or metabolized cortisol (THF+THE), or Prostaglandin (Pg)/E2.

In some embodiments, the hormone biomarkers include one or more urinary biomarkers selected from Table 15 or Table 16 (described below). In some embodiments, the supplement is administered to the subject to improve hormone levels or functions. In some embodiments, the dosage for males is selected from Table 15. In some embodiments, the dosage for females is selected from Table 16.

### MetabolicPro

The skin protects the body against environmental stressors such as solar radiation, industrial pollution, and carbon emissions. However, this protection makes it highly susceptible to premature accelerated aging due to the cumulative damage caused by these external factors. This leads to changes in cellular functions that overall affect the skin's functionality, homeostatic state, and appearance. One of the hallmarks of skin aging is alterations in metabolic processes (intrinsic factor), particularly mitochondrial dysfunction, which may lead to skin aging phenotypes such as wrinkle formation, uneven pigmentation, and decreased wound healing. Additionally, alterations in metabolic processes may also result in the loss of barrier function that increases susceptibility to infection and affects wound healing.

Nutrition is an important extrinsic factor that affects skin aging. It has been known that increased intake of carbohydrates is associated with accelerated skin aging, and may damage the skin's vital components through nonenzymatic glycation, the covalent attachment of sugar to a protein, and subsequent production of advanced glycation end products (AGEs). Glucose and fructose link the amino acids present in the collagen and elastin that support the dermis. This leads to the production of AGEs that accelerate skin aging. The intercellular lipids, such as triglycerides, cholesterol, free fatty acids, etc., in the skin's epidermis are important for the maintenance of skin moisture and elasticity. They provide proper epidermal barrier function. Disturbances in lipid quantity and composition have been implicated in several skin diseases. The fundamental skin proteins such as keratins, collagen, and elastin are made up of amino acids. Thus, amino acids are important for various integral skin functions such as wound healing, maintaining the acid-base balance and water retention in cellular layers (stratum corneum), protecting against sun damage, and maintaining the skin microbiome. Poor amino acid metabolism can result in sagging and wrinkled skin.

As a result, cellular, carbohydrate, lipid, and amino acid metabolism have profound effects on the skin's functionality and appearance. In some aspects, the methods disclosed herein further comprise a ‘MetabolicPro’ test which assesses serological and urological metabolism markers. MetabolicPro test can also assess SNPs that affect cellular, carbohydrate, lipid, and amino acid metabolism. MetabolicPro test helps draw correlations between the fundamental metabolic factors that could be affecting the skin. Based on the MetabolicPro test results, a customized skin regimen (including dietary and supplement suggestions) can be generated to improve various metabolic functions.

The results from the ‘MetabolicPro’ test can determine an individual's metabolism status and help determine the appropriate supplementation to be provided to the individual. Table 17 provides an exemplary list of biomarkers that can be tested in the ‘MetabolicPro’ test along with exemplary supplements, and dosages for males and females that can improve the biomarker levels and their associated condition.

In one aspect, the methods disclosed herein further comprise obtaining or having obtained the levels of one or more metabolism markers from serum or urine of the subject.

In some embodiments, the metabolism markers comprise one or more serum metabolism biomarkers.

In some embodiments, the serum metabolism biomarkers comprise one or more of lipid metabolism biomarkers, carbohydrate metabolism biomarkers, or cellular biomarkers.

In some embodiments, the lipid metabolism biomarkers comprise one or more of vaccenic acid, adiponectin, acylhexosylceramide (AHC), diacylglycerol (DAG), dimethyl-phosphatidylethanolamine (DMPE), or plasminogen activator inhibitor-1 (PAI-1. In some embodiments, the carbohydrate metabolism biomarkers comprise one or more of adiponectin, gastric-inhibitor-peptide (GIP), or leptin. In some embodiments, the cellular biomarkers comprise nicotinamide adenine dinucleotide (NAD).

In some embodiments, the metabolism markers comprise one or more urine metabolism biomarkers.

In some embodiments, the urine metabolism biomarkers comprise one or more of lipid metabolism biomarkers or carbohydrate metabolism biomarkers.

In some embodiments, the lipid metabolism biomarkers comprise one or more of adipate, suberate, or ethyl malonate. In some embodiments, the carbohydrate metabolism biomarkers comprise one or more of pyruvate, L-lactate, or β-hydroxybutyrate.

In some embodiments, the supplement is administered to improve metabolic functions.

In some embodiments, the metabolism marker is one or more biomarkers selected from Table 17. In some embodiments, the supplement is one or more supplements selected from Table 17. In some embodiments, the dosage for men or the dosage for women is selected from Table 17.

### NutriLite and NutriPro

Nutrients play a major role in skin health and disease. For example, vitamin A prevents UV skin damage by regulating the proliferation of epidermal keratinocytes and dermal fibroblasts. Vitamin C protects against oxidative stress by suppressing UV irradiation-triggered production of free radicals. Vitamin C also promotes cutaneous wound healing and increases epidermal moisture content, thereby improving skin hydration. Vitamin D enhances innate immunity and regulates inflammation, angiogenesis, and wound healing. Vitamin E reduces lipid peroxidation, modulates photoaging, and exhibits anti-inflammatory which is beneficial for skin health. The mineral zinc protects the skin owing to its antibacterial properties and also reduces photodamage. Copper acts as an antioxidant to stimulate the maturation of collagen and regulate collagen synthesis. Selenium, the major antioxidant of the human body, protects the skin against UV irradiation-induced oxidative stress.

Given nutrients' importance in skin health, a deficiency in the nutrients may affect their functions in the skin, resulting in various skin anomalies. In some aspects, the methods disclosed herein further comprise a ‘NutriLite’ test and a more comprehensive ‘NutriPro’ test for assessing nutrient deficiencies. Table 18 provides an exemplary list of vitamins and minerals in serum that can be examined in ‘NutriLite’ test.

In some embodiments, the method provided herein further comprises obtaining or having obtained the levels of one or more nutrients from the subject.

In some embodiments, the nutrients comprise one or more vitamins or minerals (table 13).

In some embodiments, the vitamins or minerals comprise one or more of Folate (L-5-methyltetrahydrofolate), magnesium, iron, vitamin A (retinol), vitamin D3 (cholecalciferol), vitamin E, choline, free Carnitine, vitamin B1 (thiamine diphosphate), vitamin B2 (riboflavin 5-Phosphate), vitamin B5 (pantothenic acid), vitamin B6 (pyridoxal 5-Phosphate), vitamin C (L-ascorbic acid), copper, zinc, selenium, chromium, or manganese.

The comprehensive nutrient panel ‘NutriPro’ tests the levels of various nutrients in the body along with genetic testing for predispositions due to nutrient deficiencies. Table 19 provides an exemplary list of nutrients that can be examined in NutriPro test.

In some embodiments, the nutrients tested in NutriPro test comprise one or more vitamins and minerals, amino acids, or fatty acids (table 15). The levels of these nutrients can be measured from serum, white blood cells (WBCs), or red blood cells (RBCs).

In some embodiments, the vitamins and minerals comprise vitamin A (retinol), vitamin A (beta-carotene), vitamin B1 (thiamine diphosphate), vitamin B2 (riboflavin 5-phosphate), vitamin B3 (nicotinic acid), vitamin B5 (pantothenic acid), vitamin B6, pyridoxal 5-phosphate, vitamin B7 (biotin), vitamin B12 (cyanocobalamin), vitamin C (L-ascorbic acid), vitamin D (25-OH), vitamin D3 (cholecalciferol), vitamin D (1-25 dihydroxy), vitamin E (alpha-tocopherol), vitamin K1 (phylloquinone), vitamin K2 (menaquinone-MK-7), folate (L-5-methyltetrahydrofolate), coenzyme Q10 (ubiquinone+ubiquinol), selenium, sodium, potassium, calcium, zinc, manganese, iron, magnesium, copper, chromium, myoinositol, iodine, molybdenum, phosphorus, tetrahydrobiopterin, or fluoride, copper/zinc.

In some embodiments, the amino acids comprise one or more of glutathione (oxidized), methylmalonic acid (MMA), choline, L-cysteine, L-asparagine, L-glutamine, L-serine, L-arginine, L-citrulline, L-isoleucine, L-valine, L-leucine, free carnitine, or phenylalanine.

In some embodiments, the fatty acids comprise one or more of docosahexaenoic acid (DHA), Eicosapentaenoic acid (EPA), docosapentaenoic acid (DPA), arachidonic acid (AA), or linoleic acid (LA).

In some embodiments, the method provided herein further comprises determining one or more combinations of levels of one or more of the vitamins and minerals.

In some embodiments, determining one or more combinations of levels of one or more of the vitamins and minerals further comprises obtaining a ratio of copper/zinc.

In some embodiments, the method provided herein further comprises determining one or more combinations of levels of one or more of the fatty acids.

In some embodiments, determining one or more combinations of levels of one or more of the fatty acids further comprises obtaining omega-3 total, omega-6 total, omega-3 index, or a ratio of AA/EPA.

In some embodiments, the nutrient is one or more nutrients selected from Table 18 and Table 19.

### Gut Zoomer

The skin and the gut are highly analogous to each other in purpose and functionality. For example, they are highly innervated and vascularized, and they both are crucial for immune and neuroendocrine function. The ‘gut-skin’ axis is an outcome of this resemblance. This could be the reason why gastrointestinal (GI) disturbances are often accompanied by cutaneous manifestations.

The gut microbiome exerts immuno-modulating effects on the skin. Short-chain fatty acids (SCFAs) produced from fiber fermentation in the gut are considered to be crucial in determining the predominance of certain skin microbiome profiles that subsequently influence cutaneous immune defense mechanisms. For example, S. epidermidis and P. acnes are skin commensals known to tolerate wider SCFA shifts than other flora. The gut microbiome also appears to be involved in skin barrier function by controlling the systemic modulatory effect. As a result, alterations in the gut microflora may have profound effects on the skin. Gut dysbiosis leads to increased epithelial permeability which then triggers the activation of effector T cells, disrupting their balance with immunosuppressive regulatory T cells. Pro-inflammatory cytokines further increase epithelial permeability, leading to chronic systemic inflammation. Therefore, a disturbed gut microbiome manifests in impaired skin function.

Several skin conditions such as acne, atopic dermatitis, and rosacea are linked with intestinal dysbiosis. Acne vulgaris, an inflammatory condition of the pilosebaceous unit (hair follicle, hair shaft, and sebaceous gland) is also affected by the gut microflora. Individuals with acne have altered gut composition. The hypothetical contribution of this gut flora dysregulation to acne could be the increased synthesis of fatty acids and triglycerides in the sebum leading to the aggravation of acne. Imbalance of the gut microbiota is associated with atopic dermatitis (AD). Altered gut flora and reduced SCFA production lead to a compromised intestinal barrier allowing microbes and toxins into the bloodstream. These substances, now in circulation, can target various tissues including the skin. The presence of these substances in the skin triggers immune responses, particularly the activation of Th2 immune responses. The activated Th2 immune responses can lead to tissue damage, exacerbating the symptoms of atopic dermatitis. As for rosacea, the potential consequence of the altered gut microbial metabolism is the elevated concentration of hydrogen sulfide (H2S). The increased H2S concentration may disrupt the thiol/thiol-disulfide balance in the body, causing an excess of thiol disulfides. This imbalance can result in poor protection against oxidative stress, which has been implicated in the development and progression of rosacea.

As the gut microbiome plays a crucial role in skin health and disease, its assessment may help better understand whether and how the gut may affect the skin. In some aspects, the methods disclosed herein further comprise a further ‘Gut Zoomer’ Panel to test a wide range of commensal bacteria, archaea, fungi, and viruses, along with gut metabolites that can be tested. Based on ‘Gut Zoomer’ test, gut microflora modulation can be conducted to provide therapeutic potential for skin health improvement.

In some embodiments, the method provided herein further comprises obtaining or having obtained the levels of one or more commensal microorganisms from the subject.

In some embodiments, the commensal microorganisms comprise one or more of commensal bacteria, archaea, fungi, or viruses.

In some embodiments, the method provided herein further comprises obtaining or having obtained the levels of gut metabolites from the subject.

### Example Methods for Determining Customized Skin Regimen

Disclosed herein are methods for determining customized skin regimens for individuals based on factors described herein. In various embodiments, methods can involve determining a customized skin regimen that includes particular quantities of one or more of the active element provided in any of Tables 20, 21-1, 21-2, 21-3, 21-4, 21-5, 21-6, 21-7, 21-8, 21-9. In various embodiments, the customized skin regimen includes a set of base elements, as illustrated in Table 20. Each base element has a corresponding range, and these base elements are classified into six categories e.g., polymer/solubilizing/gelling/emulsifier agents, humectant and emollient, preservative, chelating agent, penetration enhancers, and parfum. For a customized skin regime, one (or more than one base element if necessary) is selected from each category. The exact amount of each selected base element can be specified and/or adjusted if necessary.

In various embodiments, methods for determining customized skin regimens for individuals comprises analyzing one or more factors from the individual described herein. In particular embodiments, methods for determining customized skin regimens for individuals comprises analyzing one or more of (A) genetics, (B) methylation patterns, (C) demographics, (D) phenotypic assessment, and (E) skin questionnaire information.

In various embodiments, methods for determining customized skin regimens involves obtaining or having obtained genetics (e.g., genetic information from genetic testing), epigenetics information (e.g., methylation statuses), and/or phenotypic data (e.g., an image). In various embodiments, methods for determining customized skin regimens involves obtaining or having obtained genetics (e.g., genetic information from genetic testing), epigenetics information (e.g., methylation statuses), phenotypic data (e.g., an image), and/or skin questionnaire information.

In particular embodiments, methods for determining customized skin regimens involves obtaining or having obtained genetic statuses of one or more genomic locations, methylation statuses of one or more genes involved in biological processes of the skin activity category, and an image captured of the subject's skin. Genetic information can be obtained by performing methods described herein under the “Genetic Testing” heading. Epigenetics information can be obtained by performing methods described herein under the “Epigenetics” heading. Phenotypic data can be obtained by performing methods described herein under the “Phenotypic Assessment” heading. Skin questionnaire information can be obtained by performing methods described herein under the “Skin Questionnaire” heading.

In various embodiments, methods for determining customized skin regimens involves obtaining or having obtained such information for each of one or more skin activity categories, examples of which include an anti-aging and wrinkle reduction category, a lymphatic drainage category, a skin firming and elasticity category, a brightening and pigmentation category, a UV/oxidative damage/DNA/cell damage category, an anti-inflammatory and soothing category, and a hydration and skin barrier repair category. For example, methods for determining customized skin regimens involves obtaining or having obtained genetics (e.g., genetic information from genetic testing), epigenetics information (e.g., methylation statuses), and/or phenotypic data for each of one or more skin activity categories. Examples of skin activity categories, as well as active elements of those skin activity categories, are described in Tables 22-1 to 22-9. As an example, assume 7 different skin activity categories. Thus, methods may involve obtaining or having obtained obtaining or having obtained genetics (e.g., genetic information from genetic testing), epigenetics information (e.g., methylation statuses), and/or phenotypic data for each of the 7 different skin activity categories.

In various embodiments, certain information can be used across different skin activity categories whereas other information is unique to a particular skin activity category. For example, genetic statuses of different genes can be specific for a particular skin activity category, given that the biological activity of a gene can be involved in that particular skin activity category. As another example, epigenetics information for different genes can be specific for a particular skin activity category, given that the biological activity of a gene can be involved in that particular skin activity category. As another example, phenotypic information (such as an image) or questionnaire information can be used across different skin activity categories. Thus, a single image and/or a single questionnaire can be sufficient and methods need not involve obtaining multiple images and/or multiple questionnaires.

In various embodiments, methods for determining customized skin regimens involves determining one or more of a methylation score, a phenotype score, and a genetic score for the skin activity category for the subject using the one or more of genetic statuses, methylation statuses, and phenotypic information. Generally, for an example subject, the genetic score, methylation score, and phenotype score for each skin activity category may be different. Thus, a personalized customized skin regimen including specifically determined amounts of individual elements can be determined for the subject. Each of the genetic statuses, methylation statuses, and the phenotypic information are described in turn.

Referring to the genetic information, it can be used to determine a genetic score that is useful for determining quantities of active elements to be included in a customized skin regime. In various embodiments, the genetic information includes genetic statuses of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 75 or more, 100 or more, 200 or more, 300 or more, 400 or more, 500 or more, 600 or more, 700 or more, 800 or more, 900 or more, or 1000 or more genomic locations. For example, genetic statuses can be indicative of a wild type or a mutation (e.g., single nucleotide polymorphisms (SNPs), insertion, deletion, etc). In various embodiments, a genetic score can be specific for a skin activity category. Thus, for X different skin activity categories, methods can involve determining X corresponding genetic scores. Here, the genetic statuses of one or more genomic locations can be related to a particular skin activity category.

In various embodiments, a genetic score can be a quantity of genetic issues detected in an individual based on the genetic information. For example, a genetic score can be 1 genetic issue, 2 genetic issues, 3 genetic issues, 4 genetic issues, 5 genetic issues, 6 genetic issues, 7 genetic issues, 8 genetic issues, 9 genetic issues, 10 genetic issues, 11 genetic issues, 12 genetic issues, 13 genetic issues, 14 genetic issues, 15 genetic issues, 16 genetic issues, 17 genetic issues, 18 genetic issues, 19 genetic issues, 20 genetic issues, 25 genetic issues, 30 genetic issues, 35 genetic issues, 40 genetic issues, 45 genetic issues, 50 genetic issues, 55 genetic issues, 60 genetic issues, 70 genetic issues, 80 genetic issues, 90 genetic issues, 100 genetic issues, or any number of genetic issues therein.

Referring to the epigenetic information, it can be used to determine a methylation score (also referred to herein as a “methylation age”) that is useful for determining quantities of active elements to be included in a customized skin regime. In various embodiments, the epigenetic information includes methylation statuses of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 75 or more, 100 or more, 200 or more, 300 or more, 400 or more, 500 or more, 600 or more, 700 or more, 800 or more, 900 or more, or 1000 or more genomic locations. Example genomic locations (e.g., genes) for the various skin activity categories are shown in Table 7.

In various embodiments, a methylation score can be specific for a skin activity category. Thus, for X different skin activity categories, methods can involve determining X corresponding methylation scores. In various embodiments, epigenetic information can include methylation statuses of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 75 or more, 100 or more, 200 or more, 300 or more, 400 or more, 500 or more, 600 or more, 700 or more, 800 or more, 900 or more, or 1000 genes involved in biological processes of the skin activity category. In various embodiments, a methylation score is determined in accordance with Equation (1):

Methylation Age in each category=Σ(Methylation Level of Gene×Weight) (1)

Here, the category refers to one of the seven activity categories shown in Table 7, and the weight for each gene can be also found in Table 7.

Referring to the phenotypic information, it can be used to determine a phenotype score (also referred to herein as a “Phenotypic Age”) that is useful for determining quantities of active elements to be included in a customized skin regime. In various embodiments, the phenotypic information includes an image of the individual, such as an image of the individual's skin. In particular embodiments, the phenotypic information includes an image of the individual's face. In various embodiments, methods include analyzing the phenotypic information to convert the phenotypic information into a quantitative score that can be used, at least in part, to determine the phenotype score. In some embodiments, analyzing the phenotypic information can involve manually analyzing the phenotypic information and assigning quantitative scores for each of the skin activity categories. In some embodiments, analyzing the phenotypic information can involve applying an automated process (e.g., a machine learning or image recognition process) to analyze the phenotypic information and assigning quantitative scores for each of the skin activity categories. In various embodiments, the quantitative score can be between a minimum of 1 and a maximum of 10. For example, a score of 1 can be indicative of an optimal condition for the skin activity category whereas a score of 10 can be indicative of a worst condition for the skin activity category. An example of quantitative scores from an image for individual skin activity categories is shown in Table 10.

Additionally, referring to the skin questionnaire information, it can be used to determine a phenotype score (also referred to herein as a “Phenotypic Age”) that is useful for determining quantities of active elements to be included in a customized skin regime. In various embodiments, methods include analyzing the skin questionnaire information to convert provided answers into a quantitative score that can be used, at least in part, to determine the phenotype score. In various embodiments, the quantitative score can be between a minimum of 1 and a maximum of 3. In various embodiments, the quantitative score can be between a minimum of 1 and a maximum of 6. In various embodiments, the quantitative score can be between a minimum of 1 and a maximum of 8. An example of quantitative scores from a questionnaire for individual skin activity categories is shown in Table 10. Furthermore, example questions of the questionnaire are shown in Table 13 along with assigned points corresponding to particular answers.

In various embodiments, for a particular skin activity category, methods involve combining the quantitative score from the phenotypic information and the quantitative score from the questionnaire to generate the phenotype score. For example, methods can involve arithmetically combining (e.g., summing) the quantitative score from the phenotypic information and the quantitative score from the questionnaire. In various embodiments, the arithmetically combined quantitative scores are mappable to a corresponding phenotype score. For example, if the arithmetically combined quantitative score falls within a particular score range, then the score is mapped to a corresponding phenotype score for that particular score range. An example is shown in Table 11 where each points range (for a particular skin activity category) corresponds to a particular phenotype score (e.g., phenotype age as referred to in Table 11).

In various embodiments, given the methylation score, the phenotype score, and/or the genetic score, methods for determining the customized skin regimen involves determining fixed values for each of a plurality of active elements of a particular skin activity category using the corresponding scores. In various embodiments, determining the fixed values involves querying a look-up table that holds the fixed values based on a corresponding score. For example, given a genetic score for a skin activity category, methods involve accessing a list of fixed values for the active elements of the skin activity category corresponding to the genetic score. Additionally, given a methylation score, methods involve accessing a list of fixed values for the active elements of the skin activity category corresponding to the methylation score. Additionally, given a phenotype score, methods involve accessing a list of fixed values for the active elements of the skin activity category corresponding to the phenotype score.

In various embodiments, fixed values for each active element in a skin activity category can range between 0 and 3%. For example, a fixed value can be any of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%. As an example, Table 22-1 shows the fixed values for active elements in the anti-aging and wrinkle reduction skin activity category, given a genetic score (e.g., at least 1 genetic issue), a methylation score (e.g., 51-60), and a phenotype score (e.g., 41-50). Although Table 22-1 depicts one example of fixed values of active elements, one skilled in the art would understand that there are various other combinations of fixed values that can be used (e.g., between 1 and 3%) for the various active elements.

Given fixed values for each active element in a skin category for each score (e.g., genetic score, methylation score, and phenotype score), methods can involve combining certain fixed values for each active element. In various embodiments, for each active element of the plurality of active elements, methods involve combining fixed values across one or more of the methylation score, phenotype score, and genetic score to determine a percentage of the element to be included in the customized skin regimen.

For example, for an active element, methods can involve combining the fixed values across one or more, two or more, or each of the methylation score, phenotype score, and genetic score to generate an average score. For example, for an active element AE, the average score is calculated as:

Average score for AE=Average (MS_{AE}+PS_{AE})+GS_{AE}

where MS_{AE} refers to the methylation score for the active element AE, where PS_{AE} refers to the phenotype score for the active element AE, and where GS_{AE} refers to the genetic score for the active element AE.

In various embodiments, the average score for an active element can represent the quantity of the active element to be included in the customized skin regimen. In various embodiments, the average score for an active element can further undergo a scaling transformation to determine a scaled score representing the quantity of the active element to be included in the customized skin regimen. In various embodiments, the scaling factor is between 0 and 1. In various embodiments, the scaling factor is a value of 0.1, 0.15, 0.2, 0.25, 0.3, 0.35, 0.4, 0.45, 0.5, 0.55, 0.6, 0.65, 0.7, 0.75, 0.8, 0.85, 0.9, or 0.95. In particular embodiments, the scaling factor is a value of 0.75. Examples of average scores and scaled scores for each active element across various skin activity categories are shown in Tables 22-1 to 22-9.

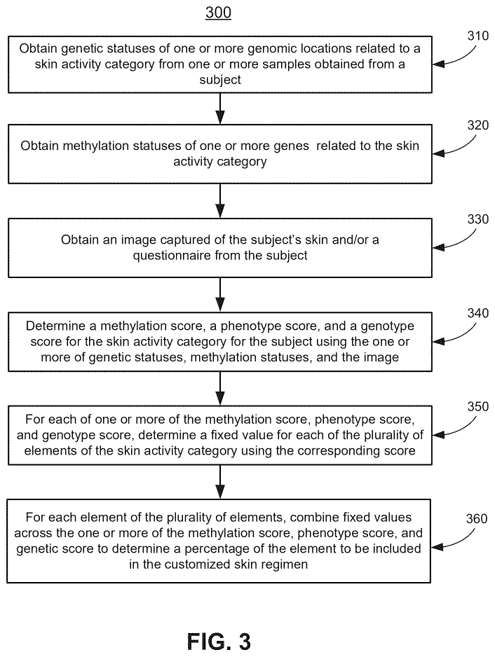

Referring now to FIG. 3, a flowchart of an exemplary method for determining a percentage of each active element in a customized skin regimen is provided, in accordance with an embodiment.

Step 310: Obtain genetic statuses of one or more genomic locations related to a skin activity category from one or more samples obtained from a subject. As used herein, a “skin activity category” refers to a category related to activity in the skin, examples of which include one or more of an anti-aging and wrinkle reduction category, a lymphatic drainage category, a skin firming and elasticity category, a brightening and pigmentation category, a UV/oxidative damage/DNA/cell damage category, an anti-inflammatory and soothing category, and a hydration and skin barrier repair category.

For example, a set of genes related to a skin activity category are checked to see whether there is any genetic issue with these genes. As disclosed herein, determining a genetic issue can involve determining deficiency predispositions at the genetic level (e.g., single nucleotide polymorphisms (SNPs), or other genetic mutations). Genetic testing for predispositions can assist in providing information about a subject's skin health. Table 4 provides exemplary skin-related polymorphisms that can be assessed and the polygenic risk associated with the polymorphisms. Genetic issues are medical or health problems that arise due to changes or mutations in an individual's DNA. These changes can affect how genes function and can lead to a variety of conditions or predispositions to certain disease. By checking the genetic statuses of these genes related to the skin activity category, it can be determined whether there are genetic issues with a particular set of genes. In some embodiments, the skin activity category is one of the seven categories described above. The one or more samples can be blood, saliva, skin biopsy or any other samples obtained from the subject for checking changes or mutations in the individual's DNA.

Step 320: Obtain methylation statuses of one or more genes related to the skin activity category. Similar to the mutation check for the genes related to the skin activity category, the methylation status for these genes can be also checked. This includes analyzing DNA from blood, saliva, buccal swabs, skin biopsy, urine samples, and the like using bisulfite conversion, sequencing, or methylation-specific PCR, pyrosequencing, among other possible methods. Based on the DNA analysis, the methylation patten for each gene related to the skin activity category can be determined, which can be used to determine the methylation score or methylation age for this skin activity category for the subject.

Step 330: Obtain an image captured of the subject's skin and a questionnaire from the subject. The image can be captured through different means and have different resolutions, but the resolutions should be high enough for phenotype evaluation. In addition, the image should be captured without using decoration tools, so that the details on the skin should more accurately reflect the subject's real condition. The questionnaire includes questions related to skin activities from different aspects, and include at least one question related to the skin activity category disclosed herein. The exemplary questions in the questionnaire can be find in Table 13.

Step 340: Determine a methylation score, a phenotype score, and a genotype score for the skin activity category for the subject using the one or more of genetic statuses, methylation statuses, and the image. In some embodiments, the methylation score or the methylation age for the skin activity category can be determined based on the methylation pattern of each gene in the category by using Equation (1) described earlier. The phenotype score can be determined based on the image evaluation and information collected from the questionnaire. For example, a first point total may be obtained from the image evaluation and a second point total can be obtained from the questionnaire, as shown in Table 10. The sum of the first point total and the second point total can be used to determine the methylation score or methylation age for that category, as can be seen in Table 11. For the genotype score, it is determined based on the number of genetic issues identified from the genes related to the skin activity category as described earlier.

Step 350: For each of one or more of the methylation score, phenotype score, and genotype score, determine a fixed value for each of the plurality of elements of the skin activity category using the corresponding score. Briefly, the determined methylation score, phenotype score, and genotype score each can be used to independently determine a desired amount or fixed value for each active element in the skin activity category. That is, based on the determined methylation score for the category, a desired amount of each active element can be determined to address methylation related concerns. Similarly, based on the determined phenotype score, another desired amount of each active element can be determined again to address phenotype related concerns, and based on the determined genotype score, yet another desired amount of each active element can be determined to address genotype related concerns. The exact amount of each active element determined from three different aspects may be different or the same.

Step 360: For each element of the plurality of elements, combine fixed values across the one or more of the methylation score, phenotype score, and genetic score to determine a percentage of the element to be included in the customized skin regimen. In other words, the exact amount of each active element determined from three different aspects in Step 350 can be combined to determine a percentage of the element to be included in the customized. In some embodiments, an average score is first computed based on the determined methylation score, phenotype score, and genetic score, which is then used to determine the percentage of the element to be included in the customized.

In this way, each active element can be determined for the exact amount to be included in the customized skin regime, which combined with the base elements can form a customized skin regimen for the subject, which can purposely address the concerns specific to the subject.

### Customized Skin Regimens

The customized skin regimen can comprise any active element provided in Tables 2, 3, 5, 6, 12, 14, 15, 16, 17, 21-1 to 21-9, or 22-1 to 22-9. In some embodiments, the skin care regimen comprises one or more topical or supplement selected from Tables 2, 3, 5, 6, 12, 14, 15, 16, 17, 21-1 to 21-9, or 22-1 to 22-9. In some embodiments, the skin care regimen comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more topical or supplement selected from Tables 2, 3, 5, 6, 12, 14, 15, 16, 17, 21-1 to 21-9, or 22-1 to 22-9.

Table 3 provides additional information about the topicals and/or supplements for use in the composition to be applied to improve skin health. Provided in Table 3 is the name of the topical and supplement, an exemplary vendor, the sequence or chemical structure of the topicals and supplements, the CAS number, and if available, the INCI name.

In various embodiments, compositions to be applied to improve skin health comprise one or more topicals and/or supplements based on presence of specific serological skin biomarkers.

In some embodiments, the serological skin biomarker is the Amino terminal propeptide of Collagen 1 (PINP), and the composition comprises one or more of vitamin C, peptides, retinoids, copper peptides, copper, collagen or collagen peptides, and omega-3 fatty acids. In some embodiments, the serological skin biomarker is the Carboxy terminal propeptide of type 1 collagen (PICP) and the composition comprises one or more of hyaluronic acids, retinoids, vitamin C, niacinamide, collagen or collagen peptides, zinc, and omega-3 fatty acids. In some embodiments, the serological skin biomarker is the Crosslinked telopeptide of type 1 collagen (ICTP), and the composition comprises one or more of retinoids, vitamin C, peptides, proline, glycine, vitamin E, vitamin C, and copper. In some embodiments, the serological skin biomarker is the Amino terminal propeptide of Collagen 3 (PIIINP), and the composition comprises one or more of alpha hydroxy acids, retinoids, vitamin C, peptides, glycine, proline, and omega-3 fatty acids. In some embodiments, the serological skin biomarker is the Matrix metalloproteinases-1 (MMP-1) and the composition comprises one or more of ginseng, green tea extract, retinoids, vitamin C, peptides, collagen or collagen peptides, vitamin E, vitamin C, and omega-3 fatty acids. In some embodiments, the serological skin biomarker is Insulin like growth factor-1 (IGF-1) and the composition comprises one or more of capsaicin, vitamin C, hyaluronic acid, zinc, vitamin C, probiotics, collagen or collagen peptides, and omega-3 fatty acids. In some embodiments, the serological skin biomarker is Basic fibroblast growth factor (FGF-2) and the composition comprises one or more of ginseng, vitamin C, hyaluronic acid, collagen or collagen peptides, resveratrol, glycine, or proline. In some embodiments, the serological skin biomarker is Matrix Metalloproteinases-12 (MMP-12) and the composition comprises one or more of vitamin C, green tea extract, silicon, ginseng, and vitamin E. In some embodiments, the serological skin biomarker is Elastin derived peptides (EDPs), and the composition comprises one or more of niacinamide, hyaluronic acid, silicon, vitamin A, and aloe vera. In some embodiments, one, two, three, four, five, six, seven, eight, nine, ten or more of the topical or supplementary compositions provided in Tables 2, 3, or 15 can be used in the skin care regimen compositions and methods described herein.

In some embodiments, the composition comprises a topical skin care composition or medication. Exemplary topicals include, but are not limited to, vitamin C, peptides, retinoids, copper peptides, hyaluronic acid, niacinamide, alpha hydroxy acids (AHA), ginseng, green tea extract, and capsaicin. In some embodiments, the composition comprises a supplemental skin care composition. Exemplary supplemental compositions include, but are not limited to, collagen and collagen peptides, vitamin C, omega-3 fatty acids, copper, zinc, omega-3 fatty acids, proline, glycine, vitamin E, curcumin, resveratrol, Eicosapentaenoic acid, Docosahexaenoic acid, vitamin D, probiotics, silicon, ginseng, aloe vera.

Peptides that can be used in the composition and method described herein are provided in Table 3. In some embodiments, the peptide is one or more of Hexapeptide-11, GHK-Cu, Palmitoyl Pentapeptide-4 (Pal-KTTKS), Palmitoyl Tetrapeptide-7 (Pal-GQPR), Dipeptide-2, Palmitoyl Pentapeptide-3, decapeptide-12, Dipeptide Diaminobutyroyl Benzylamide Diacetate, Palmitoyl Dipeptide-5 Diaminobutyroyl Hydroxythreonine, Palmitoyl Dipeptide-5 Diaminohydroxybutyrate, Tetradecyl Aminobutyroylvalylaminobutyric Urea Trifluoroacetate, Acetyl Tetrapeptide-5, Heptapeptide-4, Acetyl Hexapeptide-51 Amide, Acetyl Hexapeptide-30, Acetyl Hexapeptide-8 (Argireline), acetyl hexapeptide-3 (Argireline), Acetylarginyltryptophyl Diphenylglycine, Acetyl Hexapeptide-37, Hexapeptide-10, Acetyl Octapeptide-3, Tetrapeptide-1, Tetrapeptide-2, Acetyl Tetrapeptide-2, Acetyl Hexapeptide-38, Acetyl Dipeptide-3 Aminohexanoate, Tripeptide-10 Citrulline (Decorinyl), Acetyl Hexapeptide-49, Pentapeptide 18, Tripeptide 29, Nonapeptide-1, Tripeptide 5, Palmitoyl Tripeptide-5, Diaminopropionoyl (DAP) Tripeptide-33, beta glucan, and/or bacillus extract.

Retinoids that can be used in the composition and method described herein are provided in Table 3. In some embodiments, the retinoid is one or more of retinol, retinal, tretinoin (retinoic acid), isotretinoin, alitretinoin, adapalene, bexarotene, tazarotene, and/or Trifarotene.

Omega-3 fatty acids that can be used in the composition and method described herein are provided in Table 3. In some embodiments, the omega-3 fatty acid is one or more of Alpha-linolenic acid (ALA), Eicosapentaenoic acid (EPA), and/or Docosahexaenoic acid (DHA).

Alpha hydroxy acids (AHAs) that can be used in the composition and method described herein are provided in Table 3. In some embodiments, the Alpha hydroxy acid is one or more of glycolic acid, lactic acid, mandelic acid, and/or citric acid.

The topical and/or supplemental compositions can be formulated in an appropriate carrier or excipient. Exemplary carriers include, but are not limited to, water, Caprylyl Glycol, butylene glycol, ultra low molecular weight (ulmw) hyaluronic acid, middle molecular weight (MMW) hyaluronic acid, and dimethyl isosorbide.

Table 20 includes various base elements for the customized skin regime. For example, the base elements can include one or more of polymer/solubilizing/gelling/emulsifier agents, one or more of humectant and emollient, one or more preservative, one or more chelating agent, one or more penetration enhancers, and one or more parfum. The possible amounts or range for these base elements can be specified.

Table 21-1 to Table 21-9 further include possible active elements that can be added to the base elements for the score-based customized skin regime. As can be seen from these tables, the active elements can include certain types of peptides, one or more prebiotics and probiotics and vitamins. The peptides can be further categorized into seven different categories, namely, the anti-aging and wrinkle reduction category, the lymphatic drainage category, the skin firming and elasticity category, the brightening and pigmentation category, the UV/oxidative damage/DNA/cell damage category, the anti-inflammatory and soothing category, and the hydration and skin barrier repair category. The specific sequence of each peptide and its respective function can be further located from Table 21-1 and Table 21-7. In one specific example, palmitoyl hexapeptide-12 (Biopeptide EL) has specific function of improving skin forming and elasticity, and thus can be added as the active element to the customized skin regimen to improve the skin forming and elasticity.

The supplement or topical can be formulated at an effective amount in the skin care regimen composition. Exemplary effective concentrations for active elements, such as any active element disclosed in any of Tables 2, 3, 5, 6, 12, 14, 15, 16, 17, 21-1 to 21-9, or 22-1 to 22-9 can be 0.1-10% wt/vol or 0.1-10% wt/wt. For example, the concentration can be about 0.1%, 0.5%, 1%, 2%, 3%, 4%, 5%, 6%, 7%, 8%, 9%, or 10% wt/vol or wt/wt.

Exemplary effective amounts of active elements can also be between about 0.5-1500 mg/day. For example, effective amounts of any active element disclosed in any of Tables 2, 3, 5, 6, 12, 14, 15, 16, 17, 21-1 to 21-9, or 22-1 to 22-9 can be 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 110 mg/day, 120 mg/day, 130 mg/day, 140 mg/day, 150 mg/day, 160 mg/day, 170 mg/day, 180 mg/day, 190 mg/day, 200 mg/day, 250 mg/day, 250 mg/day, 275 mg/day, 300 mg/day, 325 mg/day, 350 mg/day, 375 mg/day, 400 mg/day, 425 mg/day, 450 mg/day, 475 mg/day, 500 mg/day, 525 mg/day, 550 mg/day, 575 mg/day, 600 mg/day, 625 mg/day, 650 mg/day, 675 mg/day, 700 mg/day, 725 mg/day, 750 mg/day, 775 mg/day, 800 mg/day, 825 mg/day, 850 mg/day, 875 mg/day, 900 mg/day, 925 mg/day, 950 mg/day, 975 mg/day, 1000 mg/day, 1025 mg/day, 1050 mg/day, 1075 mg/day, 1100 mg/day, 1125 mg/day, 1150 mg/day, 1175 mg/day, 1200 mg/day, 1225 mg/day, 1250 mg/day, 1275 mg/day, 1300 mg/day, 1325 mg/day, 1350 mg/day, 1375 mg/day, 1400 mg/day, 1425 mg/day, 1450 mg/day, 1475 mg/day, 1500 mg/day, or between about 0.5-1 mg/day, 1-5 mg/day, 5-10 mg/day, 10-15 mg/day, 15-20 mg/day, 25-30 mg/day, 35-40 mg/day, 45-50 mg/day, 55-60 mg/day, 65-70 mg/day, 60-75 mg/day 75-80 mg/day, 85-90 mg/day, 95-100 mg/day, 100-105 mg/day, 105-100 mg/day, 110-115 mg/day, 115-120 mg/day, 125-130 mg/day, 135-140 mg/day, 145-150 mg/day, 155-160 mg/day, 165-170, 170-180 mg/day, 175-80 mg/day, 185-90 mg/day, 195-200 mg/day, 200-205 mg/day, 205-210 mg/day, 210-215 mg/day, 215-220 mg/day, 225-230 mg/day, 235-240 mg/day, 245-250 mg/day, 255-260 mg/day, 265-270 mg/day, 275-280 mg/day, 285-290 mg/day, 295-300 mg/day, 300-305 mg/day, 305-310 mg/day, 310-315 mg/day, 315-320 mg/day, 325-330 mg/day, 335-340 mg/day, 345-350 mg/day, 355-360 mg/day, 365-370 mg/day, 375-380 mg/day, 385-390 mg/day, 395-400 mg/day, 400-405 mg/day, 405-410 mg/day, 410-415 mg/day, 415-420 mg/day, 425-430 mg/day, 435-440 mg/day, 445-450 mg/day, 455-460 mg/day, 465-470 mg/day, 475-480 mg/day, 485-490 mg/day, 495-500 mg/day, 500-505 mg/day, 505-510 mg/day, 510-515 mg/day, 515-520 mg/day, 525-530 mg/day, 535-540 mg/day, 545-550 mg/day, 555-560 mg/day, 565-570 mg/day, 575-580 mg/day, 585-590 mg/day, 595-600 mg/day, 600-605 mg/day, 605-610 mg/day, 610-615 mg/day, 615-620 mg/day, 625-630 mg/day, 635-640 mg/day, 645-650 mg/day, 655-660 mg/day, 665-670 mg/day, 675-680 mg/day, 685-690 mg/day, 695-700 mg/day, 700-750 mg/day, 750-800 mg/day, 85-900 mg/day, 950-1000 mg/day, 1000-1100 mg/day, 1100-1200 mg/day, 1200-1300 mg/day, 1300-1400 mg/day, or 1400-1500 mg/day, or any amount in between. Exemplary effective amounts can also be between about 1-400 mcg/day, such as 1 mcg/day, 2 mcg/day, 2.4 mcg/day, 3 mcg/day, 4 mcg/day, 5 mcg/day, 6 mcg/day, 7 mcg/day, 8 mcg/day, 9 mcg/day, 10 meg/day, 20 meg/day, 25 meg/day, 30 meg/day, 35 meg/day, 40 meg/day, 45 meg/day, 50 meg/day, 55 meg/day, 60 meg/day, 65 meg/day, 70 meg/day, 75 meg/day, 80 meg/day, 85 meg/day, 90 meg/day, 95 meg/day, 100 meg/day, 110 meg/day, 120 meg/day, 130 meg/day, 140 meg/day, 150 meg/day, 160 meg/day, 170 meg/day, 180 meg/day, 190 meg/day, 200 meg/day, 250 meg/day, 300 meg/day, 350 meg/day, or 400 meg/day or any amount in between.

### Computing Device

The methods described herein, including the methods of determining a customized skin regimen, are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

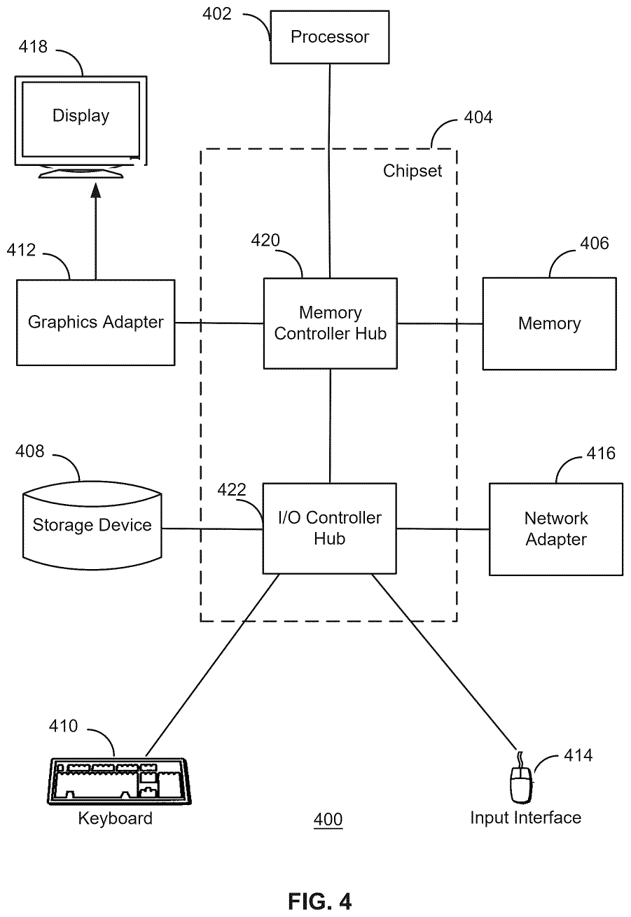

FIG. 4 illustrates an example computing device 400 for implementing system and methods described in FIGS. 1-3. In some embodiments, the computing device 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 620, and a display 418 is coupled to the graphics adapter 412. A storage device 408, an input interface 414, and network adapter 416 are coupled to the I/O controller hub 422. Other embodiments of the computing device 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of input interface, a keyboard, or some combination thereof, and is used to input data into the computing device 400. In some embodiments, the computing device 400 may be configured to receive input (e.g., commands) from the input interface 414 via gestures from the user. The graphics adapter 412 displays images and other information on the display 418. For example, the display 418 can show an indication of a treatment, such as a treatment validated by applying the cellular disease model. As another example, the display 418 can show an indication of a common chemical structure group likely contributes toward an outcome (e.g., favorable outcome or adverse outcome). As another example, the display 418 can show a candidate patient population that, through implementation of the cellular disease model, has been predicted to respond favorably to an intervention. The network adapter 416 couples the computing device 400 to one or more computer networks.

The computing device 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computing devices 400 can vary from the embodiments described herein. For example, the computing device 400 can lack some of the components described above, such as graphics adapters 412, input interface 414, and displays 418. In some embodiments, a computing device 400 can include a processor 402 for executing instructions stored on a memory 406. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### ADDITIONAL EMBODIMENTS

Disclosed herein are customized skin regimens that can help improve an individual's skin health based on a particular individual's skin profile. Such customized skin regimen can effectively provide personalized skin products, dietary, supplement, and lifestyle suggestions along with routine monitoring strategies in accordance an individual's skin profile.

In one aspect, provided herein is a method for determining a customized skin regimen for a subject, the method comprising:
i. obtaining or having obtained levels of a plurality of skin biomarkers from one or more samples obtained from the subject;
ii. obtaining or having genetic statuses of one or more genomic locations of a plurality of genes from the one or more samples obtained from the subject; and
iii. determining the customized skin regimen for the subject using both the levels of the plurality of skin biomarkers and the genetic statuses of one or more genomic locations of a plurality of genes.

In some embodiments, the skin biomarkers comprise one or more of collagens and elastins obtained from serum of the subject.

In some embodiments, the collagen biomarkers comprise one or more of amino-terminal propeptide of Collagen 1 (PINP), carboxy-terminal propeptide of type 1 collagen (PICP), crosslinked telopeptide of type 1 collagen (ICTP), amino-terminal propeptide of collagen 3 (PIIINP), matrix metalloproteinases-1 (MMP-1), tumor necrosis factor-alpha (TNF-α), insulin-like growth factor-1 (IGF-1), or basic fibroblast growth factor (FGF-2).

In some embodiments, the elastin biomarkers comprise one or more of matrix metalloproteinases-12 (MMP-12) or elastin-derived peptides (EDPs).

In some embodiments, the serum is collected using one or more of ethylenediaminetetraacetic acid (EDTA), serum separator tube (SST), dried blood spot, or ficoll tube.

In some embodiments, the levels of the plurality of skin biomarkers are obtained by using one or more of microarray or mass spectrometry.

In some embodiments, the skin biomarkers comprise one or more lipids obtained from sebum of the subject.

In some embodiments, the sebum is obtained using an absorbent medium or material.

In some embodiments, the one or more lipids comprise one or more of glycerol lipids, free fatty acids, or cholesterol.

In some embodiments, the skin biomarkers from the one or more samples obtained from the subject comprise skin biomarkers from urine of the subject.

In some embodiments, the skin biomarkers from urine of the subject comprise one or more UV damage metabolites and oxidative stress loads from the urine of the subject.

In some embodiments, the skin biomarkers comprise one or more advanced glycation end products from urine or blood of the subject.

In some embodiments, the advanced glycation end products comprise one or more of carboxymethyl lysine (CEL), carboxymethyl lysine (CML), glucosepane, or pentosidine.

In some embodiments, the skin biomarkers comprise one or more microorganisms from skin niches of the subject.

In some embodiments, the microorganisms comprise one or more of Propionibacterium acnes, Propionibacterium avidum, or Propionibacterium granulosum, optionally from forehead of the subject.

In some embodiments, the microorganisms comprise Propionibacterium acnes, optionally from cheek of the subject.

In some embodiments, the microorganisms comprise one or more of Staphylococcus epidermidis, Corynebacterium spp, or Propionibacterium acnes, optionally from nasolabial fold of the subject.

In some embodiments, the microorganisms comprise Propionibacterium acnes, optionally from chin of the subject.

In some embodiments, the microorganisms comprise Firmicutes, optionally from face of the subject.

In some embodiments, the microorganisms comprise one or more of Corynebacterium kroppenstedtiin or Staphylococcus epidermidis, optionally from cheek of the subject.

In some embodiments, the microorganisms comprise Corynebacterium kroppenstedtiin, optionally from nasolabial fold of the subject.

In some embodiments, the microorganisms comprise Staphylococcus epidermidis, optionally from eyelid of the subject.

In some embodiments, the microorganisms comprise Staphylococcus epidermidis, optionally from cheek of the subject.

In some embodiments, the microorganisms comprise Corynebacterium species, optionally from chin of the subject.

In some embodiments, the microorganisms comprise Malassezia spp, optionally from cheek of the subject.

In some embodiments, the microorganisms comprise one or more Eikenella corrodens, Cutibacterium, Micrococcus luteus, or Kocuria, optionally from cheek of the subject.

In some embodiments, the microorganisms comprise Acinetobacter, optionally from cheek of the subject.

In some embodiments, the microorganisms comprise Corynebacterium, optionally from cheek of the subject.

In some embodiments, the microorganisms comprise one or more of Corynebacterium amycolatum, Proteobacteria, Firmicutes, Bacteroidetes, or Propionibacterium, optionally from forehead of the subject.

In some embodiments, the microorganisms comprise Propionibacterium, optionally from cheek of the subject.

In some embodiments, the microorganisms comprise one or more of Corynebacterium, Lactobacillus, or Cutibacterium, optionally from face of the subject.

In some embodiments, the skin biomarkers comprise one or more hormones from urine or blood of the subject.

In some embodiments, when the subject is a male, the hormones comprise one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG).

In some embodiments, when the subject is a male, obtaining the levels of a plurality of skin biomarkers further comprises determining one or more combinations of levels of the one or more hormones.

In some embodiments, determining one or more combinations of levels of the one or more hormones comprises determining one or more of Estriol (E3)/(E1+E2), 2-OH (E1+E2)/16a-OH E1, 2-MeO E1/2-OH E1, 4-MeO E1/4-OH E1, 4-MeO E2/4-OH E2, testosterone (T)/Epi-T, b-pregnanediol/E2, cortisol/cortisone, or metabolized cortisol (THF+THE), or Prostaglandin (Pg)/E2.

In some embodiments, when the subject is a female, the hormones comprise one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5a-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG).

In some embodiments, when the subject is a female, obtaining the levels of a plurality of skin biomarkers further comprises determining one or more combinations of levels of the one or more hormones.

In some embodiments, determining one or more combinations of levels of the one or more hormones comprises determining one or more of E3/(E1+E2), 2-OH (E1+E2)/16a-OH E1, 2-MeO E1/2-OH E1, 4-MeO E1/4-OH E1, 4-MeO E2/4-OH E2, T/Epi-T, b-pregnanediol/E2, cortisol/cortisone, or metabolized cortisol (THF+THE), or Prostaglandin (Pg)/E2.

In some embodiments, the skin biomarkers comprise one or more metabolism markers from serum or urine of the subject.

In some embodiments, the metabolism markers comprise one or more serum metabolism biomarkers.

In some embodiments, the serum metabolism biomarkers comprise one or more of lipid metabolism biomarkers, carbohydrate metabolism biomarkers, or cellular biomarkers.

In some embodiments, the lipid metabolism biomarkers comprise one or more of vaccenic acid, adiponectin, acylhexosylceramide (AHC), diacylglycerol (DAG), dimethyl-phosphatidylethanolamine (DMPE), or plasminogen activator inhibitor-1 (PAI-1).

In some embodiments, the carbohydrate metabolism biomarkers comprise one or more of adiponectin, gastric-inhibitor-peptide (GIP), or leptin.

In some embodiments, the cellular biomarkers comprise nicotinamide adenine dinucleotide (NAD).

In some embodiments, the metabolism markers comprise one or more urine metabolism biomarkers.

In some embodiments, the urine metabolism biomarkers comprise one or more of lipid metabolism biomarkers or carbohydrate metabolism biomarkers.

In some embodiments, the lipid metabolism biomarkers comprise one or more of adipate, suberate, or ethyl malonate.

In some embodiments, the carbohydrate metabolism biomarkers comprise one or more of pyruvate, L-lactate, or β-hydroxybutyrate.

In some embodiments, the skin biomarkers comprise one or more nutrients from the subject.

In some embodiments, the nutrients comprise one or more vitamins or minerals.

In some embodiments, the vitamins or minerals comprise one or more of Folate (L-5-methyltetrahydrofolate), magnesium, iron, vitamin A (retinol), vitamin D3 (cholecalciferol), vitamin E, choline, free Carnitine, vitamin B1 (thiamine diphosphate), vitamin B2 (riboflavin 5-Phosphate), vitamin B5 (pantothenic acid), vitamin B6 (pyridoxal 5-Phosphate), vitamin C (L-ascorbic acid), copper, zinc, selenium, chromium, or manganese.

In some embodiments, the nutrients comprise one or more vitamins and minerals, amino acids, or fatty acids.

In some embodiments, the vitamins and minerals comprise vitamin A (retinol), vitamin A (beta-carotene), vitamin B1 (thiamine diphosphate), vitamin B2 (riboflavin 5-phosphate), vitamin B3 (nicotinic acid), vitamin B5 (pantothenic acid), vitamin B6, pyridoxal 5-phosphate, vitamin B7 (biotin), vitamin B12 (cyanocobalamin), vitamin C (L-ascorbic acid), vitamin D (25-OH), vitamin D3 (cholecalciferol), vitamin D (1-25 dihydroxy), vitamin E (alpha-tocopherol), vitamin K1 (phylloquinone), vitamin K2 (menaquinone-MK-7), folate (L-5-methyltetrahydrofolate), coenzyme Q10 (ubiquinone+ubiquinol), selenium, sodium, potassium, calcium, zinc, manganese, iron, magnesium, copper, chromium, myoinositol, iodine, molybdenum, phosphorus, tetrahydrobiopterin, or fluoride, copper/zinc.

In some embodiments, the method further comprises further comprising determining one or more combinations of levels of one or more of the vitamins and minerals.

In some embodiments, determining one or more combinations of levels of one or more of the vitamins and minerals further comprises obtaining a ratio of copper/zinc.

In some embodiments, the amino acids comprise one or more of glutathione (oxidized), methylmalonic acid (MMA), choline, L-cysteine, L-asparagine, L-glutamine, L-serine, L-arginine, L-citrulline, L-isoleucine, L-valine, L-leucine, free carnitine, or phenylalanine.

In some embodiments, the fatty acids comprise one or more of docosahexaenoic acid (DHA), Eicosapentaenoic acid (EPA), docosapentaenoic acid (DPA), arachidonic acid (AA), or linoleic acid (LA).

In some embodiments, the method further comprises obtaining the levels of a plurality of skin biomarkers by determining one or more combinations of levels of one or more of the fatty acids.

In some embodiments, determining one or more combinations of levels of one or more of the fatty acids further comprises obtaining omega-3 total, omega-6 total, omega-3 index, or a ratio of AA/EPA.

In some embodiments, the skin biomarkers comprise one or more commensal microorganisms from the subject.

In some embodiments, the commensal microorganisms comprise one or more of commensal bacteria, archaea, fungi, or viruses.

In some embodiments, the skin biomarkers further comprise gut metabolites from the subject.

In some embodiments, the method further comprises obtaining or having obtained one or more demographics of the subject; and determining the customized skin regimen for the subject based on the demographics of the subject.

In some embodiments, the demographics comprise one or more of an age or gender of the subject.

In some embodiments, the method further comprises:
iv. obtaining or having obtained an image of a face from the subject;
v. grading severities of one or more phenotypic parameters for the face of the subject based on the image; and
vi. determining the customized skin regimen for the subject based on the severity of the one or more phenotypic parameters.

In some embodiments, the phenotypic parameters comprise one or more of wrinkles, periorbital-around eyes (puffy eyes), nasolabial folds (laugh lines), worry lines, uneven skin tone/pigmentation, or age spots (liver spots).

In some embodiments, the method further comprises obtaining or having obtained answers to a skin questionnaire from the subject; and determining the customized skin regimen for the subject based on the answers from the subject.

In some embodiments, the questionnaire comprises one or more questions related to skin goals, type of skin, sun exposure, or skin proneness to external factors.

In some embodiments, the method further comprises administering the customized skin regimen to the subject. For example, the customized skin regimen can be applied topically to the skin of the subject.

In another aspect, provided herein are customized skin regimens, wherein the customized skin regimen is determined according to a method disclosed herein.

In some embodiments,
i. a skin biomarker is the Amino terminal propeptide of Collagen 1 (PINP), and the composition comprises one or more of vitamin C, peptides, retinoids, copper peptides, copper, collagen or collagen peptides, and/or omega-3 fatty acids;
ii. a skin biomarker is the Carboxy terminal propeptide of type 1 collagen (PICP) and the composition comprises one or more of hyaluronic acids, retinoids, vitamin C, niacinamide, collagen or collagen peptides, zinc, and/or omega-3 fatty acids;
iii. a skin biomarker is the Crosslinked telopeptide of type 1 collagen (ICTP), and the composition comprises one or more of retinoids, vitamin C, peptides, proline, glycine, vitamin E, vitamin C, and/or copper;
iv. a serological skin biomarker is the Amino terminal propeptide of Collagen 3 (PIIINP), and the composition comprises one or more of alpha hydroxy acids, retinoids, vitamin C, peptides, glycine, proline, and/or omega-3 fatty acids;
v. a skin biomarker is the Matrix metalloproteinase-1 (MMP-1) and composition comprises one or more of ginseng, green tea extract, retinoids, vitamin C, peptides, collagen or collagen peptides, vitamin E, vitamin C, and/or omega-3 fatty acids;
vi. a skin biomarker is Insulin like growth factor-1 (IGF-1) and the composition comprises one or more of capsaicin, vitamin C, hyaluronic acid, zinc, vitamin C, probiotics, collagen or collagen peptides, and/or omega-3 fatty acids;
vii. a skin biomarker is Basic fibroblast growth factor (FGF-2) and the composition comprises one or more of ginseng, vitamin C, hyaluronic acid, collagen or collagen peptides, resveratrol, glycine, and/or proline;
viii. a skin biomarker is Matrix Metalloproteinase-12 (MMP-12) and the composition comprises one or more of vitamin C, green tea extract, silicon, ginseng, and/or vitamin E;
ix. a skin biomarker is Elastin derived peptides (EDPs), and the composition comprises one or more of niacinamide, hyaluronic acid, silicon, vitamin A, and/or aloe vera.

In some embodiments, the peptide is one or more of Hexapeptide-11, GHK-Cu, Palmitoyl Pentapeptide-4 (Pal-KTTKS), Palmitoyl Tetrapeptide-7 (Pal-GQPR), Dipeptide-2, Palmitoyl Pentapeptide-3, decapeptide-12, Dipeptide Diaminobutyroyl Benzylamide Diacetate, Palmitoyl Dipeptide-5 Diaminobutyroyl Hydroxythreonine, Palmitoyl Dipeptide-5 Diaminohydroxybutyrate, Tetradecyl Aminobutyroylvalylaminobutyric Urea Trifluoroacetate, Acetyl Tetrapeptide-5, Heptapeptide-4, Acetyl Hexapeptide-51 Amide, Acetyl Hexapeptide-30, Acetyl Hexapeptide-8 (Argireline), acetyl hexapeptide-3 (Argireline), Acetylarginyltryptophyl Diphenylglycine, Acetyl Hexapeptide-37, Hexapeptide-10, Acetyl Octapeptide-3, Tetrapeptide-1, Tetrapeptide-2, Acetyl Tetrapeptide-2, Acetyl Hexapeptide-38, Acetyl Dipeptide-3 Aminohexanoate, Tripeptide-10 Citrulline (Decorinyl), Acetyl Hexapeptide-49, Pentapeptide 18, Tripeptide 29, Nonapeptide-1, Tripeptide 5, Palmitoyl Tripeptide-5, Diaminopropionoyl (DAP) Tripeptide-33, beta glucan, and/or bacillus extract.

In some embodiments, the retinoid is one or more of retinol, retinal, tretinoin (retinoic acid), isotretinoin, alitretinoin, adapalene, bexarotene, tazarotene, and/or Trifarotene.

In some embodiments, the omega-3 fatty acid is one or more of Alpha-linolenic acid (ALA), Eicosapentaenoic acid (EPA), and/or Docosahexaenoic acid (DHA).

In some embodiments, the Alpha hydroxy acid is one or more of glycolic acid, lactic acid, mandelic acid, and/or citric acid.

In some embodiments, wherein
i. a polymorphism is a SNP associated with melanin synthesis and the composition comprises one or more of Hydroquinone, Retinoids, Azelaic acid, Kojic acid, Cysteamine, Lignin peroxidase, Niacinamide, Ellagic acid, L-Cystine, L-Glutathione, Polypodium leucotomos, Melatonin, Arbutin, and/or Vitamin C;
ii. a polymorphism is a SNP associated with Moisturization and the composition comprises one or more of Vitamin E, Coenzyme Q10, Vitamin F, Kumazasa extract, Magnesium ascorbyl phosphate, Hyaluronic acid, Magnesium oil, collagen, ceramides, hyaluronan, procyanidin, probiotics, Galactooligosaccharides, Alpha-linolenic acid, and/or Gamma-linolenic acid;
iii. a polymorphism is a SNP associated with Skin Inflammation and the composition comprises one or more of Coenzyme Q10, Methylsulfonylmethane, Retinoid, Vitamin E, Vitamin C, Curcumin, Green tea or green tea extract, and/or Omega 3 fatty acids;
iv. a polymorphism is a SNP associated with Melanocyte damage and the composition comprises one or more of Coenzyme Q10, Methylsulfonylmethane, Retinoid, Vitamin E, Vitamin D, Zinc, Vitamin C, Curcumin, Green tea or green tea extract, and/or Omega 3 fatty acids;
v. a polymorphism is a SNP associated with Collagen degradation and the composition comprises one or more of Retinoids, Vitamin C, Vitamin E, Tretinoin, Collagen peptides, Red ginseng, Aloe vera extract, Hyaluronic acid, Ginkgo biloba, and/or Soy;
vi. a polymorphism is a SNP associated with Elastin synthesis and the composition comprises one or more of Retinoid, Vitamin C, Collagen or collagen peptides, Hyaluronic acid, Dexpanthenol, Vitamin E, Vitamin A, and/or Zinc;
vii. a polymorphism is a SNP associated with Antioxidants and the composition comprises one or more of Vitamin C, Coenzyme Q10, Polyphenols, Vitamin E, Curcumin, Vitamin C, Vitamin E, Beta carotene, Flavanols, zinc, Lutein, Lycopene, Silymarin, and/or Green tea or green tea extract;
viii. a polymorphism is a SNP associated with Vitamin A deficiency and the composition comprises one or more of Retinol, Vitamin E, Resveratrol, Baicalin, B-vitamins, Vitamin C, Epigallocatechin gallate, Collagen, Nicotinamide riboside, Nicotinamide mononucleotide, Coenzyme Q10, and/or Curcumin;
ix. a polymorphism is a SNP associated with Vitamin B6 deficiency and the composition comprises one or more of B-vitamins, Vitamin B6, Coenzyme Q10, Methylsulfonylmethane; Retinoids, and Vitamin E, Curcumin, Green tea or green tea extract, Omega 3 fatty acids, and/or Vitamin C;
x. a polymorphism is a SNP associated with Vitamin B12 deficiency and the composition comprises one or more of Retinol, Vitamin E, Resveratrol, Baicalin, B-vitamins, Vitamin B12, Vitamin C, Curcumin, Epigallocatechin gallate, Collagen, Nicotinamide riboside, Nicotinamide mononucleotide, and/or Coenzyme Q10;
xi. a polymorphism is a SNP associated with Vitamin C deficiency and the composition comprises one or more of Vitamin C, Coenzyme Q10, Polyphenols, Vitamin E, Curcumin, Beta carotene, Flavanols, zinc, Lutein, Lycopene, Silymarin, and/or Green tea or green tea extract;
xii. a polymorphism is a SNP associated with Vitamin D deficiency and the composition comprises one or more of Vitamin D, Retinol, Vitamin E, Resveratrol, Baicalin, B-vitamins, Vitamin C, Curcumin, Epigallocatechin gallate, Collagen, Nicotinamide riboside, Nicotinamide mononucleotide, and/or Coenzyme Q10;
xiii. a polymorphism is a SNP associated with Folate deficiency and the composition comprises one or more of Folate, Retinol, Vitamin E, Resveratrol, Baicalin, B-vitamins, Vitamin C, Curcumin, Epigallocatechin gallate, Collagen, Nicotinamide riboside, Nicotinamide mononucleotide, and Coenzyme Q10; and/or
xiv. a polymorphism is a SNP associated with Omega fatty acid deficiency and the composition comprises one or more of DHA (Docosahexaenoic acid), EPA, Coenzyme AL, Methylsulfonylmethane, Retinoid, Curcumin, Green tea or green tea extract and/or Vitamin E.

In some embodiments, a phenotypic parameter is wrinkles, Periorbital-around eyes (puffy eyes), Nasolabial folds (Laugh lines), worry lines, Uneven Skin tone/Pigmentation, and/or Age spots (Liver spots) and the composition comprises one or more of Tretinoin, Zeaxanthin, Retinol, Retinoids, Vitamin C, Hyaluronic acid, Caffeine, Phytonadione, Vitamin E, Astaxanthin, Hyaluronic acid, Peptides, Niacinamide, Azelaic acid, Soybean extract, Kojic acid, Aloesin, and/or Turmeric.

In some embodiments, the customized skin regimen further comprises one or more of Zeaxanthin, Collagen, Vitamin C, Resveratrol, Vitamin E, Vitamin C, Hyaluronic acid, Glutathione, Niacinamide, and/or Coenzyme Q10.

In some embodiments, a skin questionnaire question is:
i. UV and the composition comprises one or more of Zinc oxide, titanium oxide, Polypodium leucotomos, Vitamin E, Vitamin C, and/or Niacinamide;
ii. Anti-wrinkling and the composition comprises one or more of Tretinoin, Zeaxanthin, Retinol, Vitamin C, Hyaluronic acid, Collagen, Resveratrol, and/or Vitamin E;
iii. Anti-pigmentation and the composition comprises one or more of Niacinamide, Azelaic acid, Vitamin C, Soybean extract, Retinoids, Kojic acid, Vitamin E, Aloesin, Turmeric, and/or Glutathione
iv. Moisturization and the composition comprises one or more of Hyaluronic acid, Glycerin, Shea butter, Jojoba oil, Hyaluronan, Ceramides, and Collagen;
v. Radiant complexion and the composition comprises one or more of Vitamin C and/or Niacinamide,
vi. Dry skin and the composition comprises Glycerin;
vii. Combinational skin and the composition comprises Hyaluronic acid;
viii. Acne-prone skin and the composition comprises Benzoyl peroxide;
ix. Sun exposure and the composition comprises Zinc oxide and/or Titanium dioxide;
x. Sensitive skin—stinging or burning and the composition comprises Niacinamide
xi. Sun and Humidity and the composition comprises one or more of Vitamin E, Zinc oxide, Astaxanthin, and/or Omega-3 fatty acids
xii. Wind and cold and the composition comprises one or more of Petroleum jelly, Omega-3 fatty acids, and/or Probiotics.

In some embodiments, a hormone is:
i. one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG); or
ii. one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5α-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG); and
iii. the composition comprises one or more of Calcium D-glucarate, Green Tea, Maca DIM (3,3′-diindolylmethane), Black Cohosh, Ginger, Cranberry, Milk Thistle, Hops, Red clover, Damiana, Chaste Tree Berry Extract, Vitamin B2, Fenugreek seed, Ashwagandha, Vitamin D3, Zinc, Tribulus, Shatavari, Zinc, Shatavari, DHA, EPA, pine nuts, Licorice Root Extract, Oregano, Curcumin, Sodium, Rhodiola rosea, Vitamin B6, Vitamin B12, Magnesium, Folate, Iodine, Selenium, Potassium, Magnesium, Melatonin, Huang-qin, St. John's-wort, SAMe (S-adenosyl methionine), and/or Trimethylglycine (betaine).

In some embodiments, a metabolic biomarker is one or more of Vaccenic acid, Adiponectin, Acylhexosylceramide (AHC), Dimethyl-phosphatidylethanolamine (DMPE), PAI-1, GIP, Leptin, NAD, Adipate, Suberate, Ethyl malonate, Pyruvate, L-lactate, and/or β-hydroxybutyrate and the supplemental or topical is one or more of Conjugated Linoleic Acid, DHA, EPA, Vitamin D, Curcumin, Berberine Extract, Resveratrol, Vitamin B3, Vitamin B6, Vitamin E, Choline, Vitamin C, Tryptophan, Nicotinamide riboside, Resveratrol, Quercetin, Vitamin B2, L-carnitine, Vitamin B1, Vitamin B12, CoQ10 (Coenzyme Q10), Alpha lipoic acid, and/or Chromium.

In some embodiments, the customized skin regimen further comprises an excipient or carrier.

In some embodiments, the excipient or carrier is one or more of water, Caprylyl Glycol, butylene glycol, ultra low molecular weight (ulmw) hyaluronic acid, middle molecular weight (MMW) hyaluronic acid, and/or dimethyl isosorbide

In another aspect, provided herein are customized skin regimens, wherein the customized skin regimen comprises one or more supplement or topical medication, or a supplement and a topical medication.

In some embodiments, the one or more topical medication and/or supplement comprise one or more of B-vitamins, Vitamin B1, Vitamin B2, Vitamin B3, Vitamin B6, Vitamin B12, vitamin C, vitamin D, vitamin E, vitamin F, peptides, retinoids, copper peptides, copper, zinc, collagen or collagen peptides, omega-3 fatty acids, hyaluronic acids, niacinamide, zinc, proline, glycine, vitamin E, alpha hydroxy acids, ginseng, green tea or green tea extract, capsaicin, zinc, probiotics, resveratrol, glycine, proline, green tea or green tea extract, silicon, niacinamide, silicon, vitamin A, aloe vera, Hydroquinone, Azelaic acid, Kojic acid, Cysteamine, Lignin peroxidase, Ellagic acid, L-Cystine, L-Glutathione, Polypodium leucotomos, Melatonin, Arbutin, Coenzyme Q10, Vitamin F, Kumazasa extract, Magnesium ascorbyl phosphate, Magnesium oil, ceramides, hyaluronan, procyanidin, probiotics, Galactooligosaccharides, Alpha-linolenic acid, Gamma-linolenic acid, Methylsulfonylmethane, Curcumin, Red ginseng, Aloe vera extract, Dexpanthenol, Polyphenols, Beta carotene, Flavanols, zinc, Lutein, Lycopene, Silymarin, Baicalin, Epigallocatechin gallate, Nicotinamide riboside, Nicotinamide mononucleotide, Zeaxanthin, Caffeine, Phytonadione, Astaxanthin, Soybean extract, Aloesin, Turmeric, Glycerin, Shea butter, Jojoba oil, Benzoyl peroxide, Conjugated Linoleic Acid, Berberine Extract, Quercetin, Calcium D-glucarate, Zinc oxide and/or Titanium dioxide.

In some embodiments, the peptide is one or more of Hexapeptide-11, GHK-Cu, Palmitoyl Pentapeptide-4 (Pal-KTTKS), Palmitoyl Tetrapeptide-7 (Pal-GQPR), Dipeptide-2, Palmitoyl Pentapeptide-3, decapeptide-12, Dipeptide Diaminobutyroyl Benzylamide Diacetate, Palmitoyl Dipeptide-5 Diaminobutyroyl Hydroxythreonine, Palmitoyl Dipeptide-5 Diaminohydroxybutyrate, Tetradecyl Aminobutyroylvalylaminobutyric Urea Trifluoroacetate, Acetyl Tetrapeptide-5, Heptapeptide-4, Acetyl Hexapeptide-51 Amide, Acetyl Hexapeptide-30, Acetyl Hexapeptide-8 (Argireline), acetyl hexapeptide-3 (Argireline), Acetylarginyltryptophyl Diphenylglycine, Acetyl Hexapeptide-37, Hexapeptide-10, Acetyl Octapeptide-3, Tetrapeptide-1, Tetrapeptide-2, Acetyl Tetrapeptide-2, Acetyl Hexapeptide-38, Acetyl Dipeptide-3 Aminohexanoate, Tripeptide-10 Citrulline (Decorinyl), Acetyl Hexapeptide-49, Pentapeptide 18, Tripeptide 29, Nonapeptide-1, Tripeptide 5, Palmitoyl Tripeptide-5, Diaminopropionoyl (DAP) Tripeptide-33, beta glucan, and/or bacillus extract.

In some embodiments, the retinoid is one or more of retinol, retinal, tretinoin (retinoic acid), isotretinoin, alitretinoin, adapalene, bexarotene, tazarotene, and/or Trifarotene.

In some embodiments, the omega-3 fatty acid is one or more of Alpha-linolenic acid (ALA), Eicosapentaenoic acid (EPA), and/or Docosahexaenoic acid (DHA).

In some embodiments, the Alpha hydroxy acid is one or more of glycolic acid, lactic acid, mandelic acid, and/or citric acid.

In some embodiments, the customized skin regimen further comprises an excipient or carrier.

In some embodiments, the excipient or carrier is one or more of water, Caprylyl Glycol, butylene glycol, ultra low molecular weight (ulmw) hyaluronic acid, middle molecular weight (MMW) hyaluronic acid, and/or dimethyl isosorbide.

### EXAMPLES

### Example 1: Calculating a Customized Skin Regimen for an Individual

Table 22-1 to Table 22-9 together illustrate an example of the specific amount of each active element to be included in the customized skin regime, which include the specific amount of each peptide, each prebiotic and probiotic, and each vitamin. As used in this example, an “active element” includes any element included in a customized skin regimen that can improve the skin of an individual. An “active element” can include any of a protein, a carbohydrate, a sugar, a lipid, a nucleic acid, an organism (e.g., a unicellular bacteria, virus, or toxin), a nutrient, and a vitamin.

In the following, Table 22-1 is utilized to illustrate the calculation of an amount of an active element in the anti-aging and wrinkle reduction category, to be included in the customized skin regime. Here, three scores were calculated including genotype score (referred to as “genotype”), methylation score (referred to as “methylation age”), and phenotype score (referred to as “phenotype age”).

Specifically, the methylation score for the category was calculated by analyzing methylation patterns in the genes in Table 7 labelled as “anti-aging and wrinkle reduction” (e.g., CDKN2A, COL1A1, MMP1, SIRT1). Associated weights were assigned to each of the genes as shown in Table 7, and the methylation age (or methylation score) was calculated as the sum of the methylation level of each of the genes, weighted by the corresponding weights. The phenotype score for the category was calculated by capturing images of the individual's face, and by manually annotating the image for anti-aging and wrinkle reduction. A first point total for anti-aging and wrinkle reduction was assigned based on the annotated image (between 1-10 as shown in Table 10). Furthermore, a second point total was assigned for anti-aging and wrinkle reduction based on the patient questionnaire (between 0-3 for this category, or 0-6 or 0-8 for other categories as shown in Table 10). Specific questions and the points assigned are shown in Table 13. After determining the first point total and the second point total for each of the seven categories, the first and second point totals were combined and phenotype age was assigned according to the combined points. Specifically, according to Table 11, the points range and corresponding phenotype age are shown. In general, the higher the combined point total, the larger the corresponding age for each category, although the specific point range for each phenotype age for each category may be different.

In the next, the genotype score, methylation age, and phenotype age were used to determine corresponding fixed values for each of the anti-aging and wrinkle reduction elements. Reference is now made to Table 22-1. Here, at least 1 genetic issue was identified for the genes included in this category, leading to the corresponding fixed values shown for each of the elements (e.g., 0.50% for palmitoyl pentapeptide-3 (Matrixyl), and so on). Additionally, the individual was determined to have a methylation age of 51-60, leading to corresponding fixed values shown in Table 22-1 for each of the elements (e.g., 2% for palmitoyl pentapeptide-3 (Matrixyl), and so on). Additionally, the individual was determined to have a phenotype age of 41-50, leading to corresponding fixed values shown in Table 22-1 for each of the elements (e.g., 2% for palmitoyl pentapeptide-3 (Matrixyl), and so on).

Next, the average score for each element was determined. Specifically, for each element, the percentages were combined across the genotype score, the methylation age, and the phenotype age to generate an average score, as described earlier. For example, for palmitoyl pentapeptide-3 (Matrixyl), the average score is calculated as:

Average score for palmitoyl pentapeptide-3 (Matrixyl)=Average (2%+2%)+0.50%=2.5%.

Finally, the average score was scaled to determine the final percentage of eah element, included in the category, to be added to the customized skin regime. Here, the scaling value for palmitoyl pentapeptide-3 (Matrixyl) was 60/80 and therefore, the final scaled percentage was calculated as:

Scaled value=60/80*2.5%=1.9%.

Thus, Palmitoyl Pentapeptide-3 (Matrixyl) was included in the customized skin regimen at 1.9% wt/wt %.

For other elements included in the anti-aging and wrinkle reduction category, the similar process was repeated to obtain the scaled percentage, which is the amount included in the customized skin regime. The eventual value for each element can be located in Table 22-1.

The process was further repeated for all other active elements in other categories e.g., as shown in Table 22-2 for the elements in the skin firming and elasticity category, Table 22-3 for the elements in the hydration and skin barrier repair category, Table 22-4 for the elements in the anti-inflammatory and soothing category, Table 22-5 for the elements in the brightening and pigmentation category, Table 22-6 for the elements in the lymphatic draining category, and Table 22-7 for the elements in the UV/Oxidative Damage/DNA/Cell damage category. For the Table 22-8 (pre and probiotics), and Table 22-9 (Vitamins), the determined methylation age was used to determine the average score, which was further scaled to obtain the scaled value. Here, the scaled value represents the eventual amount added to the customized skin regimen for each active element.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents, and patent applications cited within the body of the instant specification are hereby incorporated by reference in their entirety, for all purposes.

TABLE 1

| | |
|---|---|
| Serological markers | Significance |

Collagen biomarkers (synthesis and degradation)

| | |
|---|---|
| (a) Amino terminal propeptide of Collagen 1 (PINP) | PINP is associated with collagen synthesis. |
| (b) Carboxy terminal propeptide of type 1 collagen (PICP) | PICP is an indicator of collagen synthesis in skin recovery. |
| (c) Crosslinked telopeptide of type 1 collagen (ICTP) | ICTP is an indicator of collagen degradation. |
| (d) Amino terminal propeptide of Collagen 3 (PIIINP) | PIIINP is an indicator of collagen turnover. |
| (e) Matrix metalloproteinases-1 (MMP-1) | MMP-1 belongs to the class of collagenases that |
| | degrade type 1 and 3 collagen. |
| (f) Tumor necrosis factor alpha (TNF-α) | TNF-α induces collagen degradation by activation of collagenases. |
| (g) Insulin like growth factor-1 (IGF-1) | IGF-1 stimulates fibroblast proliferation, inhibits collagenases |
| | and stimulates collagen expression |
| (h) Basic fibroblast growth factor (FGF-2) | FGF-2 stimulates the synthesis of type 1 collagen and induces |
| | fibroblast proliferation and activation. |

Elastin biomarkers (degradation)

| | |
|---|---|
| (a) Matrix Metalloproteinases-12 (MMP-12) | MMP-12 degrades skin elastin. |
| (b) Elastin derived peptides (EDPs) | EDPs are formed as a result of elastin degradation |
| | by proteases like elastase or MMPs. |

TABLE 2

| Serological marker | Topicals | Supplements |
|---|---|---|
| Amino terminal | 1. Vitamin C-improves collagen synthesis and provides | 1. Collagen peptides-supports collagen synthesis and through |
| propeptide of | strength and structure to the skin. | its antioxidant activity protecting the skin from free radicals |
| Collagen 1 (PINP) | 2. Peptides-strengthen the skin barrier, increase water | and UV radiation. |
| | retention, and stimulate collagen production, while also | 2. Vitamin C-improves collagen synthesis by the enzymatic |
| | improving the appearance of wrinkles in aging skin. | reaction that converts pro-collagen to collagen. |
| | 3. Retinoids-increase the production of new collagen | 3. Omega-3 fatty acids-help to reduce inflammation which |
| | fibers, while inhibiting the enzymes that break down | prevents collagen damage and premature aging and improves |
| | existing collagen | dry and dull skin. |
| | 4. Copper peptides-activate growth factors, such as | 4. Copper-stimulates fibroblasts and collagen production, |
| | TGF-beta, that stimulate collagen and extracellular | elastin fiber components, crosslinks collagen, promotes |
| | matrix protein production by fibroblasts, ultimately | collagen fibril formation, and protects against oxidative |
| | improving skin health. | damage. |
| Carboxy terminal | 1. Hyaluronic acids-retain skin moisture, promote | 1. Collagen-increases density and diameter of collagen fibrils, |
| propeptide | collagen synthesis, and have antioxidant properties that | stimulates fibroblasts and extracellular matrix protein |
| of type 1 | protect the skin from oxidative stress, which can | production |
| collagen (PICP) | damage collagen. | 2. Vitamin C-improves collagen synthesis amd provides |
| | 2. Retinoids-increase the production of new collagen | strength and structure to the skin. |
| | fibers, while inhibiting the enzymes that break down | 3. Zinc-is essential for collagen synthesis and its anti- |
| | existing collagen. | inflammatory help with wound healing and keratinocyte |
| | 3. Vitamin C-improves collagen synthesis by the | survival. |
| | enzymatic reaction that converts pro-collagen to | 4. Omega-3 fatty acid-reduces inflammation that can damage |
| | collagen. | collagen, improve the skin barrier, and enhance moisture |
| | 4. Niacinamide-supports key enzymes involved in | retention by increasing the production of ceramides. |
| | collagen synthesis and increases collagen and protein | |
| | production in the dermis, leading to a reduction in fine | |
| | lines and wrinkles. | |
| Crosslinked | 1. Retinoids-increase the production of new collagen | 1. Proline-involved in collagen synthesis, thus, it supports the |
| telopeptide of | fibers, while inhibiting the enzymes that break down | skin by preventing the appearance of fine lines and wrinkles. |
| type 1 | existing collagen. | 2. Glycine-the main amino acid in collagen, reduces the |
| collagen (ICTP) | 2. Vitamin C-improves collagen synthesis by the | formation of wrinkles by increasing collagen production and |
| | enzymatic reaction that converts pro-collagen to | maintaining epidermal barrier homeostasis. |
| | collagen. | 3. Vitamin E-protects the skin from free radicals that can |
| | 3. Peptides-stimulate collagen production and improve | damage collagen and other proteins thereby preserving skin |
| | skin hydration, strengthen the skin barrier, increase | integrity. |
| | water retention, and reduce the appearance of wrinkles | 4. Vitamin C-improves collagen synthesis by the enzymatic |
| | when applied topically. | reaction that converts pro-collagen to collagen |
| | | 5. Copper-stimulates dermal fibroblasts and collagen |
| | | production, elastin fiber components, promotes collagen |
| | | fibril formation, crosslinks collagen, and elastin matrices, |
| | | and protects against oxidative damage, all of which maintain |
| | | skin health. |
| Amino terminal | 1. Alpha hydroxy acids (AHAs)-exfoliate the skin and | 1. Vitamin C-stimulates collagen synthesis by the enzymatic |
| propeptide of | improve collagen synthesis by increasing the activity of | reaction that converts pro-collagen to collagen |
| Collagen 3 (PIIINP) | fibroblasts and by activating growth factors such as | 2. Glycine-is the main amino acid in collagen, and it reduces |
| | TGF-beta. | the formation of wrinkles by increasing collagen production |
| | 2. Retinoids-produce new collagen fibers, while | and maintaining epidermal barrier homeostasis. |
| | inhibiting the enzymes that break down existing | 3. Proline-is involved in collagen synthesis, thus, it supports |
| | collagen. | the skin by preventing the appearance of fine lines and |
| | 3. Vitamin C-improves collagen synthesis by the | wrinkles. |
| | enzymatic reaction that converts pro-collagen to | 4. Omega-3 fatty acids-reduce inflammation that can damage |
| | collagen. | collagen, improve the skin barrier, and enhance moisture |
| | 4. Peptides-stimulate collagen production and improve | retention by increasing the production of ceramides. |
| | skin hydration, strengthen the skin barrier, increase | |
| | water retention, and reduce the appearance of wrinkles | |
| | when applied topically. | |
| Matrix | 1. Ginseng-increases type I collagen production in the | 1. Collagen-increases density and diameter of collagen fibrils, |
| metalloproteinase-1 | skin's extracellular matrix by regulating the expression | stimulates fibroblasts and extracellular matrix protein |
| (MMP-1) | of MMP-1 and TIMP-1 enzymes, which can reduce | production that enhances skin health. |
| | collagen breakdown and improve skin health. | 2. Vitamin E-protects the skin from free radicals that can |
| | 2. Green tea extract-is rich in polyphenols, which | damage collagen and other proteins, thereby preserving skin |
| | stimulate collagen synthesis by fibroblasts and promote | integrity. |
| | healthy and elastic skin. | 3. Vitamin C-improves collagen synthesis by the enzymatic |
| | 3. Retinoids-increase the production of new collagen | reaction that converts pro-collagen to collagen |
| | fibers, while inhibiting the enzymes that break down | 4. Omega-3 fatty acids-reduce inflammation that can damage |
| | existing collagen. | collagen, improve the skin barrier, and enhance moisture |
| | 4. Vitamin C-improves collagen synthesis by the | retention by increasing the production of ceramides. |
| | enzymatic reaction that converts pro-collagen to | |
| | collagen | |
| | 5. Peptides-stimulate collagen production and improve | |
| | skin hydration, strengthen the skin barrier, increase | |
| | water retention, and reduce the appearance of wrinkles | |
| | when applied topically. | |
| Tumor necrosis | 1. Peptides-stimulate collagen production and improve | 1. Curcumin-inhibits NF-κB activity and decreases the level |
| factor alpha | skin hydration, strengthen the skin barrier, increase | of TNFα, which induces collagen degradation by activation |
| (TNF-α) | water retention, and reduce the appearance of wrinkles | of collagenases. |
| | when applied topically. | 2. Resveratrol-reduces inflammatory factors such as AP-1 |
| | 2. Green tea extract-is rich in polyphenols, which | and NF-KB that damage collagen and neutralizes free |
| | stimulate collagen synthesis by fibroblasts and promote | radicals and protects the skin against their damaging effects. |
| | healthy and elastic skin. | 3. Eicosapentaenoic acid-preserves collagen and stimulates |
| | 3. Vitamin C-improves collagen synthesis by the | its production by reducing the levels of pro-inflammatory |
| | enzymatic reaction that converts pro-collagen to | cytokines in the body. |
| | collagen | 4. Docosahexaenoic acid-decreases the level of TNFα, which |
| | 4. Retinoids-increase the production of new collagen | induces collagen degradation and might further promote |
| | fibers, while inhibiting the enzymes that break down | collagen production. |
| | existing collagen. | 5. Vitamin D-prevents collagen degradation by inhibiting |
| | | MMP activity, promoting collagen synthesis, and reducing |
| | | inflammation. |
| Insulin like | 1. Capsaicin-stimulates the release of calcitonin gene- | 1. Zinc-promotes collagen synthesis, wound healing, and |
| growth factor-1 | related peptide (CGRP) and the production of insulin- | keratinocyte survival due to its role in proliferation, |
| (IGF-1) | like growth factor-I (IGF-I), leading to improved skin | differentiation, and anti-inflammatory properties. |
| | elasticity. | 2. Vitamin C-improves collagen synthesis by the enzymatic |
| | 2. Vitamin C-improves collagen synthesis by the | reaction that converts pro-collagen to collagen |
| | enzymatic reaction that converts pro-collagen to | 3. Probiotics-can reduce skin sensitivity, support the skin's |
| | collagen | immune function, and improve cutaneous immune |
| | 3. Hyaluronic acid-promotes skin hydration and | homeostasis, thereby improving skin homeostasis. |
| | elasticity while supporting healthy IGF-1 levels | 4. Collagen-increases density and diameter of collagen fibrils, |
| | through extracellular matrix protein synthesis. | stimulates fibroblasts and extracellular matrix protein |
| | | production that enhances skin health. |
| | | 5. Omega-3 fatty acid-reduces inflammation that can damage |
| | | collagen, improves the skin barrier, and enhances moisture |
| | | retention by increasing the production of ceramides. |
| Basic fibroblast | 1. Ginseng-increases collagen production and reduces | 1. Collagen-increases density and diameter of collagen fibrils, |
| growth factor | collagen breakdown, by the activity of MMP-1 and | stimulates fibroblasts and extracellular matrix protein |
| (FGF-2) | TIMP-1, enzymes. | production that enhances skin health. |
| | 2. Vitamin C-improves collagen synthesis by the | 2. Resveratrol-stimulates fibroblast growth factor 2 (FGF-2) |
| | enzymatic reaction that converts pro-collagen to | production, which promotes the growth of skin cells, |
| | collagen | including collagen-producing fibroblasts. |
| | 3. Hyaluronic acid-promotes skin hydration and | 3. Vitamin C-improves collagen synthesis by the enzymatic |
| | elasticity while supporting healthy IGF-1 levels | reaction that converts pro-collagen to collagen |
| | through extracellular matrix protein synthesis. | 4. Glycine-is the main amino acid in collagen, and it reduces |
| | | the formation of wrinkles by increasing collagen production |
| | | and maintaining epidermal barrier homeostasis. |
| | | 5. Proline-is involved in collagen synthesis, thus, it supports |
| | | the skin by preventing the appearance of fine lines and |
| | | wrinkles. |
| Matrix | 1. Vitamin C-plays a crucial role in skin collagen | 1. Silicon-plays a critical role in the synthesis of collagen and |
| Metalloproteinase- | synthesis which increases fibroblasts and extracellular | the activation of hydroxylation enzymes which are essential |
| 12 | matrix proteins and decreases metalloproteinase, | in forming the collagen network, leading to improved skin |
| (MMP-12) | thereby helping to maintain skin elasticity. | strength and elasticity. |
| | 2. Green tea extract-contains polyphenols that have | 2. Vitamin C-plays a crucial role in skin collagen synthesis |
| | been shown to inhibit MMP activity and protect the | which increases fibroblasts and extracellular matrix proteins |
| | skin from UV damage. | and decreases metalloproteinase, thereby helping to maintain |
| | | skin elasticity. |
| | | 3. Ginseng-can increase type I procollagen synthesis and |
| | | downregulate MMP protein levels which prevent the skin |
| | | from wrinkling. |
| | | 4. Vitamin E-reduces free radicals that can damage collagen |
| | | and other proteins in the skin, thereby, preserving the skin |
| | | integrity. |
| Elastin derived | 1. Niacinamide-converted to NADPH, a precursor vital | 1. Silicon-plays a critical role in the synthesis of collagen and |
| peptides | for collagen synthesis, resulting in improved skin | the activation of hydroxylation enzymes which are essential |
| (EDPs) | elasticity.″ | in forming the collagen network, leading to improved skin |
| | 2. Hyaluronic acid-promotes skin hydration and | strength and elasticity. |
| | elasticity while supporting healthy IGF-1 levels through | 2. Vitamin A-protects collagen and elastin from degradation, |
| | extracellular matrix protein synthesis. | supports keratinocyte proliferation, and restrains trans |
| | | epidermal water loss all of which improves skin elasticity. |
| | | 3. Aloe vera-reduces skin aging by promoting collagen and |
| | | elastin fiber synthesis, improving elasticity, moisturizing, |
| | | and tightening pores. |

TABLE 3

| | | | | | | | |
|---|---|---|---|---|---|---|---|
| | Brand or | | | | | | |
| | Trademark | | Sequence/ | | | | |
| Name | Name | Vendor | Formula | Category | Description | CAS No | INCI name |

peptides

| | | | | | | | |
|---|---|---|---|---|---|---|---|
| Hexapeptide- | N/A | Active Peptide | Phe-Val-Ala- | Anti wrinkle | Enhance skin | 161258-30-6 | |
| 11 | | Company | Pro-Phe-Pro | | firmness and | | |
| | | | | | elasticity. Stimulates | | |
| | | | | | the production of | | |
| | | | | | collagen, reduces | | |
| | | | | | fine lines and | | |
| | | | | | wrinkles. Brightens | | |
| | | | | | complexion. Fights | | |
| | | | | | signs of aging and | | |
| | | | | | increases water | | |
| | | | | | retention capacity. | | |
| GHK-Cu | N/A | Active Peptide | Gly-His-Lys | AntiSagging | Copper is an | 49557-75-7 | |
| | | Company | (glycyl-L- | | important trace | 89030-95-5 | |
| | | | histidyl-L-lysine) | | element that plays a | (Cu complex) | |
| | | | | | key factor in the | | |
| | | | | | production of | | |
| | | | | | collagen and elastin; | | |
| | | | | | GHK-Cu can be a | | |
| | | | | | natural supplement | | |
| | | | | | for copper in the | | |
| | | | | | connective tissue | | |
| | | | | | synthesis | | |
| Palmitoyl | N/A | Active Peptide | Pal-Lys-Thr-Thr- | Anti wrinkle | Used for wrinkle | 214047-00-4 | |
| Pentapeptide-4 | | Company | Lys-Ser | | prevention and | | |
| (Pal-KTTKS) | | | | | repair, eye contour | | |
| | | | | | care, and mature | | |
| | | | | | skincare. It has | | |
| | | | | | comparable efficacy | | |
| | | | | | to Retinol without | | |
| | | | | | causing damage to | | |
| | | | | | the skin barrier. | | |
| Palmitoyl | N/A | Active Peptide | Pal-Gly-Gln-Pro- | Anti- | Skin-restoring | 221227-05-0 | |
| Tetrapeptide-7 | | Company | Arg | inflammation | ingredient; noted for | | |
| (Pal-GQPR) | | | | | its soothing ability | | |
| | | | | | due to interruption | | |
| | | | | | of factors within | | |
| | | | | | skin that lead to | | |
| | | | | | signs of irritation | | |
| | | | | | (including from | | |
| | | | | | exposure to UVB | | |
| | | | | | light) and loss of | | |
| | | | | | firmness. | | |
| Dipeptide-2 | N/A | Active Peptide | Trp-Val | Around eye | Considered a skin | 24587-37-9 | |
| | | Company | | | conditioning | | |
| | | | | | ingredient, smaller | | |
| | | | | | size allows it to | | |
| | | | | | easily penetrate | | |
| | | | | | skin's uppermost | | |
| | | | | | layers where it can | | |
| | | | | | assist with skin | | |
| | | | | | renewal and | | |
| | | | | | replenishment. | | |
| Palmitoyl | N/A | CellBone | Pal-Lys-Thr-Ser | AntiSagging | stimulates the | 214047-00-4 | |
| Pentapeptide-3 | | Technology | | | development of | | |
| | | Inc | | | collagen, elastin, | | |
| | | | | | and other | | |
| | | | | | components which | | |
| | | | | | moisturize and | | |
| | | | | | support the dermal | | |
| | | | | | matrix. It also has | | |
| | | | | | been shown to not | | |
| | | | | | cause any adverse | | |
| | | | | | side effects, making | | |
| | | | | | it as effective as | | |
| | | | | | retinol, but less | | |
| | | | | | irritating. | | |
| decapeptide- | N/A | CellBone | Tyr-Arg-Ser- | Photoaging | Decapeptide-12 is | 137665-91-9 | |
| 12 | | Technology | Arg-Lys-Tyr- | and | an oligopeptide | | |
| | | Inc | Ser-Ser-Trp-Tyr | Brightening | known for its anti- | | |
| | | | | peptides | tyrosinase | | |
| | | | | | properties. | | |
| | | | | | Tyrosinase catalyzes | | |
| | | | | | the production of | | |
| | | | | | melanin and other | | |
| | | | | | pigments. | | |
| | | | | | Decapeptide-12 | | |
| | | | | | helps to inhibit the | | |
| | | | | | production of | | |
| | | | | | tyrosinase and fade | | |
| | | | | | dark spots, called | | |
| | | | | | hyper-pigmentation, | | |
| | | | | | caused by sun | | |
| | | | | | damage or PIH | | |
| | | | | | (post-inflammatory | | |
| | | | | | hyper- | | |
| | | | | | pigmentation). | | |
| Dipeptide | SYN ®-AKE | DMS | B-Ala-Pro-Dab- | Anti wrinkle | SYN ®-AKE | 823202-99-9 | Glycerin, Aqua, |
| Diamino- | | | NHBzl | | enables the face to | (acetate) | Dipeptide |
| butyroyl | | | | | relax, reducing | | Diaminobutyroyl |
| Benzylamide | | | | | wrinkles but without | | Benzylamide |
| Diacetate | | | | | losing the ability to | | Diacetate |
| | | | | | help people express | | |
| | | | | | themselves. Helps | | |
| | | | | | delaying the | | |
| | | | | | appearance of | | |
| | | | | | wrinkles, with age- | | |
| | | | | | freezing effects. | | |
| Palmitoyl | SYN ®-TACKS | DMS | palmitoyl-Lys- | AntiSagging | Improve lymphatic | 883558-31-4 | Palmitoyl |
| Dipeptide-5 | | | Val-Dab-Thr-OH | | circulation; used to | | Dipeptide-5 |
| Diamino- | | | | | combat dark and | | Diaminobutyroyl |
| butyroyl | | | | | puffy under-eye | | Hydroxythreonine, |
| Hydroxy- | | | | | circles. | | Palmitoyl |
| threonine | | | | | | | Dipeptide-5 |
| | | | | | | | Diaminohydroxy- |
| | | | | | | | butyrate |
| Palmitoyl | SYN ®-TACKS | DMS | palmitoyl-Lys- | AntiSagging | Improve lymphatic | 794590-33-3 | Palmitoyl |
| Dipeptide-5 | | | Val-Dab-Thr-OH | | circulation; used to | | Dipeptide-5 |
| Diamino- | | | | | combat dark and | | Diaminobutyroyl |
| hydroxy- | | | | | puffy under-eye | | Hydroxythreonine, |
| butyrate | | | | | circles. | | Palmitoyl |
| | | | | | | | Dipeptide-5 |
| | | | | | | | Diaminohydroxy- |
| | | | | | | | butyrate |
| Tetradecyl | SYN ®-HYCAN | DMS | Tetradecyl | | | 934368-60-2 | Glycerin, Aqua, |
| Amino- | | | aminocarbonyl- | | | | Tetradecyl |
| butyroyl | | | Dab-Val-Dab | | | | Aminobutyroyl- |
| valylamino- | | | | | | | valylaminobutyric |
| butyric | | | | | | | Urea |
| Urea | | | | | | | Trifluoroacetate, |
| Trifluoro- | | | | | | | Magnesium |
| acetate | | | | | | | Chloride |
| Acetyl | EYESERYL ® | Lubrizol | Ac-ß-Ala-His- | Around eye, | A tetrapeptide with | 820959-17-9 | Glycerin, Aqua, |
| Tetrapeptide-5 | peptide solution | | Ser-His-OH | puffy eyes | a demonstrated | | Acetyl |
| | G | | | | efficacy against | | Tetrapeptide-5 |
| | | | | | puffy eyebags; | | |
| | | | | | Decreased | | |
| | | | | | appearance of dark | | |
| | | | | | circles | | |
| Heptapeptide- | FENSEBIOME ™ | Lubrizol | Ac-Glu-Glu- | | | 1459206-66-6 | Aqua, Acetyl |
| 4 | peptide solution | | Met-Gln-Arg- | | | | Heptapeptide-4, |
| | | | Arg-Ala | | | | Caprylyl Glycol |
| Acetyl | JUVELEVEN ™ | Lubrizol | Ac-Glu-Glu-Leu- | | | | Butylene Glycol, |
| Hexapeptide- | peptide solution | | Lys-Tye-Val | | | | Aqua, Acetyl |
| 51 Amide | | | | | | | Hexapeptide-51 |
| | | | | | | | Amide |
| Acetyl | INYLINE ® | Lubrizol | Ac-Arg-Asp- | Anti wrinkle | Targets the post- | | Acetyl |
| Hexapeptide- | peptide solution | | Glu-Leu-Tyr | | synaptic pathway; | | Hexapeptide-30, |
| 30 | | | | | Disrupts AChR | | Arginine, |
| | | | | | clustering in vitro; | | Caprylyl Glycol, |
| | | | | | Attenuates | | Aqua |
| | | | | | expression wrinkles | | |
| Acetyl | PD280 | Lubrizol, | Ac-Glu-Glu- | Photoaging | Reprogramming the | 616204-22-9 | Aqua, Disodium |
| Hexapeptide-8 | REPROAGE ™ | Active Peptide | Met-Gln-Arg- | and | skin for a youthful | | Phosphate, Acetyl |
| (Argireline) | peptide solution | Company or | Arg-NH2 | Brightening | appearance; Support | | Hexapeptide-8, |
| | | CellBone | | peptides, | skin's collagen and | | Sodium |
| | | Technology | | anti-wrinkle | preserve a firmer | | Phosphate, |
| | | Inc | | | appearance. | | Caprylyl Glycol |
| | | | | | Neurotransmitter- | | |
| | | | | | inhibiting peptide | | |
| Acetylarginyl- | Relistase ® | Lubrizol | Ac-Arg-Trp-Phg- | | | 1334583-93-5 | Glycerin, |
| tryptophyl | peptide | | Phg-OH | | | | Acetylarginyl- |
| Diphenyl- | | | | | | | tryptophyl |
| glycine | | | | | | | Diphenylglycine |
| Acetyl | DIFFUPORINE ® | Lubrizol | Ac-Ser-Pro-Ala- | | | 1447824-16-9 | Acetyl |
| Hexapeptide- | peptide solution | | Gly-Gly-Pro- | | | | Hexapeptide-37 |
| 37 | | | NH2 | | | | |
| Hexapeptide- | SERILESINE ® | Lubrizol | H-Ser-Ile-Lys- | | | 146439-94-3 | aqua, |
| 10 | peptide solution | | Val-Ala-Val-OH | | | | Hexapeptide-10 |
| | GC | | | | | | |
| Acetyl | SNAP-8 ™ | Lubrizol | Ac-glu-glu-met- | Anti wrinkle | Muscle relaxant, | 868844-74-0 | Water (Aqua), |
| Octapeptide-3 | peptide solution | | gln-arg-arg-ala- | | reduces muscle | | Acetyl |
| | C | | asp-NH2 | | contraction. | | Octapeptide-3, |
| | | | | | | | Caprylyl Glycol |
| Tetrapeptide-1 | Uplevity ™ e-Lift | Lubrizol | leucine, proline, | AntiSagging | Creator of | | Water (Aqua), |
| | peptide | | threonine, and | | functional elastin, | | Tetrapeptide-1, |
| | | | valine; Leu-Pro- | | Aids in restructuring | | Caprylyl Glycol |
| | | | Thr-Val | | the dermis | | |
| Acetyl | ADIFYLINE ® | Lubrizol | Ac-Ser-Val-Val- | AntiSagging | Stimulates | 1400634-44-7 | Butylene Glycol, |
| Hexapeptide- | peptide solution | | Val-Arg-Thr- | | adipogenesis in | | Water (Aqua), |
| 38 | | | NH2 or Ac-DL- | | vitro; Increases | | Acetyl |
| | | | Ser-DL-Val-DL- | | cheek volume | | Hexapeptide-38 |
| | | | Val-DL-Val-DL- | | | | |
| | | | Arg-DL-xiThr- | | | | |
| | | | OH | | | | |
| Acetyl | BODYFENSINE ® | Lubrizol | Ac-Arg-Ala- | | | 40968-45-4 | Butylene Glycol, |
| Dipeptide-3 | peptide solution | | Ahx-OH | | | | Aqua, Acetyl |
| Amino- | | | | | | | Dipeptide-3 |
| hexanoate | | | | | | | Aminohexanoate |
| Tripeptide-10 | DECORINYL ® | Lubrizol | H-Lys-α-Asp- | AntiSagging | Collagen | 960531-53-7 | Water (Aqua), |
| Citrulline | functional | | Ile-Citrulline- | | organization to | | Lecithin, |
| (Decorinyl) | ingredient | | NH2 | | restore skin | | Tripeptide-10 |
| | | | | | suppleness; Helps | | Citrulline, |
| | | | | | improve | | Carbomer, |
| | | | | | fibrillogenesis in | | Triethanolamine, |
| | | | | | vitro; Controls fibril | | Caprylyl Glycol, |
| | | | | | dimensions in vitro; | | Phenoxyethanol |
| | | | | | Gives suppleness to | | |
| | | | | | the skin | | |
| Acetyl | DELISENS ™ | Lubrizol | Ac-Phe-Phe-Trp- | | | 1969409-70-8 | Butylene Glycol, |
| Hexapeptide- | peptide solution | | Phe-His-Val-OH | | | | Water (Aqua), |
| 49 | | | | | | | Citric Acid, |
| | | | | | | | Acetyl |
| | | | | | | | Hexapeptide-49 |
| Acetyl | Uplevity ™ e-Lift | Lubrizol | Ac-Lys-Asp-Val- | | | 757942-88-4 | |
| Tetrapeptide-2 | peptide | | Tyr | | | | |
| Pentapeptide | N/A | 001Chemical | H-D-Tyr-D-Ala- | Around eye | ability to reduce | 64963-01-5 | |
| 18 | | | Gly-Phe-Leu-OH | | signs of aging, | | |
| | | | | | including | | |
| | | | | | minimizing the | | |
| | | | | | appearance of | | |
| | | | | | expression lines and | | |
| | | | | | visibly reducing | | |
| | | | | | wrinkle depth. | | |
| acetyl | Argireline | Active Peptide | Ac-Glu-Glu- | Anti wrinkle | Support skin's | 616204-22-9 | |
| hexapeptide-3 | | Company or | Met-Gln-Arg- | | collagen and | | |
| | | CellBone | Arg-NH2 | | preserve a firmer | | |
| | | Technology | | | appearance. | | |
| | | Inc | | | Neurotransmitter- | | |
| | | | | | inhibiting peptide | | |
| acetyl | Argireline | Active Peptide | Ac-Glu-Glu- | Anti wrinkle | Support skin's | 616204-22-9 | |
| hexapeptide-8 | | Company or | Met-Gln-Arg- | | collagen and | | |
| | | CellBone | Arg-NH2 | | preserve a firmer | | |
| | | Technology | | | appearance. | | |
| | | Inc | | | Neurotransmitter- | | |
| | | | | | inhibiting peptide | | |
| Tripeptide 29 | N/A | 001Chemical, | H-Gly-Pro-Hyp- | Anti wrinkle | Increase collagen | 2239-67-0 | |
| | | RR Scientific | OH | | synthesis and repair | | |
| | | | | | skin. | | |
| Nonapeptide-1 | N/A | BLD Pharm, | H-Met-Pro-D- | Photoaging | Inhibits the | 158563-45-2 | |
| | | RR Scientific | Phe-Arg-D-Trp- | and | production of | | |
| | | | Phe-Lys-Pro- | Brightening | melanin, the | | |
| | | | Val-NH2 | peptides | pigment that gives | | |
| | | | | | skin its color. Can | | |
| | | | | | help brighten the | | |
| | | | | | complexion, even | | |
| | | | | | out skin tone, and | | |
| | | | | | reduce the | | |
| | | | | | appearance of dark | | |
| | | | | | spots and age spots | | |
| Tripeptide 5, | N/A | RR Scientific | Lys-Val-Lys or | AntiSagging | Visibly reduces fine | 623172-56-5 | |
| Palmitoyl | | | palmitoyl-Lys- | | lines and wrinkles | | |
| Tripeptide-5 | | | Val-Lys-OH | | (including deep | | |
| | | | | | wrinkles), | | |
| | | | | | encourages a firmer | | |
| | | | | | look and feel, helps | | |
| | | | | | reduce rough texture | | |
| DAP— | Preventhelia ® | Lubrizol | {Dap}-Ala-Pro- | Photoaging | Prevent skin cell | 1199495-15-2 | Diaminopropionoyl |
| Tripeptide 33, | | | His | and | damages induced | | Tripeptide-33, |
| Diaminopro- | | | | Brightening | directly or indirectly | | Caprylyl Glycol |
| pionoyl | | | | peptides | by UV irradiation. | | |
| Tripeptide-33 | | | | | | | |
| Beta Glucan | N/A | THE BioTek | beta-D-gluco- | Anti-oxidant | Beta-glucan offers | 9041-22-9 | |
| | | | hexopyranosyl- | | antioxidant | | |
| | | | (1->4)- | | properties and has | | |
| | | | hexopyranosyl- | | been shown to help | | |
| | | | (1->4)- | | visibly minimize | | |
| | | | hexopyranose | | signs of aging/sun | | |
| | | | | | damage (including | | |
| | | | | | reducing the look of | | |
| | | | | | wrinkle depth and | | |
| | | | | | rough, uneven tone). | | |
| Bacillus | N/A | DermaElements, | | Anti- | Bacillus extract has | | |
| Extract | | Cat. No: DE48 | | inflammation | soothing and | | |
| | | | | | hydrating properties | | |
| | | | | | due to its ability to | | |
| | | | | | help strengthen | | |
| | | | | | skin's microbiome, | | |
| | | | | | enabling this | | |
| | | | | | “invisible barrier” to | | |
| | | | | | reduce water loss | | |
| | | | | | and better maintain | | |
| | | | | | surface hydration | | |

Other topical and supplement compositions

| | | | | | |
|---|---|---|---|---|---|
| butylene | N/A | Sigma-Aldrich | 1,3-Butylene | Absorption | 107-88-0 |
| glycol | | | Glycol | enhancer | |
| ulmw | N/A | Sigma-Aldrich | | Moisture | 9067-32-7 |
| hyaluronic | | | | | |
| acid | | | | | |
| MMW | N/A | Sigma-Aldrich | | Moisture and | 9067-32-7 |
| hyaluronic | | | | gelling agent | |
| acid | | | | | |
| dimethyl | N/A | Sigma-Aldrich | | Absorption | 5306-85-4 |
| isosorbide | | | | enhancer | |
| vitamin C | N/A | Sigma-Aldrich | | | 50-81-7 |
| viramin E | N/A | Sigma-Aldrich | | | 10191-41-0 |
| vitamin D | N/A | Sigma-Aldrich | | | 67-97-0 |
| retinoids | N/A | Sigma-Aldrich | retinol | | 68-26-8 |
| | N/A | Sigma-Aldrich | retinal | | 116-31-4 |
| | N/A | Sigma-Aldrich | tretinoin (retinoic | | 302-79-4 |
| | | | acid) | | |
| | N/A | Sigma-Aldrich | isotretinoin | | 4759-48-2 |
| | N/A | Sigma-Aldrich | alitretinoin | | 5300 03 8 |
| | N/A | Sigma-Aldrich | adapalene, | | 106685-40-9 |
| | N/A | Sigma-Aldrich | bexarotene | | 153559-49-0 |
| | N/A | Sigma-Aldrich | tazarotene | | 118292-40-3 |
| | N/A | Sigma-Aldrich | Trifarotene | | 895542-09-3 |
| collagen | N/A | Sigma-Aldrich | | | 9007-34-5 |
| Omega-3 fatty | N/A | Sigma-Aldrich | Alpha-linolenic | | 463-40-1 |
| acids | | | acid (ALA) | | |
| | N/A | Sigma-Aldrich | Eicosapentaenoic | | 10417-94-4 |
| | | | acid (EPA) | | |
| | N/A | Sigma-Aldrich | Docosahexaenoic | | 6217-54-5 |
| | | | acid (DHA) | | |
| | N/A | Sigma-Aldrich | | | |
| copper | N/A | Sigma-Aldrich | | | 7440-50-8 |
| hyaluronic | N/A | Sigma-Aldrich | | | 9004-61-9 |
| acid | | | | | |
| niacinamide | N/A | Sigma-Aldrich | | | 98-92-0 |
| zinc | N/A | Sigma-Aldrich | | | 7440-66-6 |
| proline | N/A | Sigma-Aldrich | | | 147-85-3 |
| glycine | N/A | Sigma-Aldrich | | | |
| alpha hydroxy | N/A | Sigma-Aldrich | glycolic acid, | | 79-14-1 |
| acids (AHAs) | N/A | Sigma-Aldrich | lactic acid | | 79-33-4 |
| | N/A | Sigma-Aldrich | mandelic acid | | 611-72-3 |
| | N/A | Sigma-Aldrich | citric acid. | | 77-92-9 |
| ginseng | N/A | Sigma-Aldrich | | | 50647-08-0 |
| green tea | N/A | Sigma-Aldrich | | | 84650-60-2 |
| extract | | | | | |
| curcumin | N/A | Sigma-Aldrich | | | 458-37-7 |
| Resveratrol | N/A | Sigma-Aldrich | | | 501-36-0 |
| Capsaicin | N/A | Sigma-Aldrich | | | 404-86-4 |
| Probiotics | N/A | Various | | | |
| | | vendors | | | |
| Silicon | N/A | Sigma-Aldrich | | | 7440-21-3 |
| Aloe vera | N/A | Sigma-Aldrich | | | 85507-69-3 |

TABLE 4

| | | | Genotype value | | | Polygenic risk factor |
|---|---|---|---|---|---|---|
| SNP Category | rs ID | Outcome | Wild | Het | Mutant | (PRF) |
| Melanin | rs 1426654 | Differences in melanin production leading to pigmentation | 1.0 | 1.1 | 1.2 | (rs1426654 + rs16891982 + |
| synthesis | (SLC24A5 gene) | | | | | rs1042602 + rs1393350 + |
| | rs16891982 | Accumulation of melanin-containing melanosomes in the | 1.0 | 1.1 | 1.2 | rs6058017 + rs74653330 + |
| | (SLC45A2 gene) | epidermis leading to pigmentation | | | | rs1393350 + rs1805008 + |
| | rs1042602 | Accumulation of melanin-containing melanosomes in the | 1.0 | 1.1 | 1.2 | rs1015362 + rs4911414 + |
| | (TYR gene) | epidermis leading to pigmentation | | | | rs12203592 + rs1805007 + |
| | rs1393350 | Accumulation of melanin-containing melanosomes in the | 1.0 | 1.1 | 1.2 | rs1805009) |
| | (TYR gene) | epidermis leading to pigmentation | | | | 13 |
| | rs6058017 | Alteration in the gene regulating melanogenesis leading to | 1.0 | 1.1 | 1.2 | |
| | (ASIP gene) | pigmentation | | | | |
| | rs74653330 | Alteration in the gene regulating melanogenesis leading to | 1.0 | 0.9 | 0.8 | |
| | (OCA2 gene) | pigmentation | | | | |
| | rs 1393350 | Altered skin sensitivity to the sun leading to skin burn and | 1.0 | 0.9 | 0.8 | |
| | (TYR gene) | freckles | | | | |
| | rs1805008 | Overproduction of melanin leading to pigmentation | 1.0 | 1.1 | 1.2 | |
| | (MC1R gene) | | | | | |
| | rs1015362 | Altered skin sensitivity to the sun leading to skin burns and | 1.0 | 0.9 | 0.8 | |
| | (ASIP gene) | freckles | | | | |
| | rs4911414 | Altered skin sensitivity to the sun leading to skin burns and | 1.0 | 0.9 | 0.8 | |
| | (ASIP gene) | freckles | | | | |
| | rs 12203592 | Poor activation of TYR enzyme required for melanin | 1.0 | 0.9 | 0.8 | |
| | (IRF4 gene) | synthesis | | | | |
| | rs1805007 | Increased production of phaeomelanin, resulting in light | 1.0 | 0.9 | 0.8 | |
| | (MC1R gene) | skin and poor tanning ability | | | | |
| | rs 1805009 | Increased production of phaeomelanin, resulting in light | 1.0 | 0.9 | 0.8 | |
| | (MC1R gene) | skin and poor tanning ability | | | | |
| Moisturization | rs17553719 | Increased water loss from the skin leading to reduced skin | 1.0 | 1.1 | 1.2 | (rs17553719 + rs61816761 + |
| | (AQP3 gene) | moisturization capacity | | | | rs138726443 + rs150597413) |
| | rs61816761 | Shortage of natural moisturizing factors and impaired skin | 1.0 | 1.1 | 1.2 | 4 |
| | (FLG gene) | barrier function affecting skin moisturization capacity | | | | |
| | rs138726443 | Shortage of natural moisturizing factors and impaired skin | 1.0 | 1.1 | 1.2 | |
| | (FLG gene) | barrier function affecting skin moisturization capacity | | | | |
| | rs 150597413 | Shortage of natural moisturizing factors and impaired skin | 1.0 | 1.1 | 1.2 | |
| | (FLG gene) | barrier function affecting skin moisturization capacity | | | | |
| Skin | rs20541 | Elevated IL-13 levels leading to skin inflammation and | 1.0 | 1.1 | 1.2 | (rs20541 + rs2201841 + |
| Inflammation | (IL-13 gene) | lesions | | | | rs1801133 + rs610604 + |
| | rs2201841 | Elevated IL-23 levels leading to skin inflammation and | 1.0 | 1.1 | 1.2 | rs17728338 + rs1800795) |
| | (IL23R gene) | lesions | | | | 6 |
| | rs1801133 | Elevated homocysteine levels leading to immuno- | 1.0 | 1.1 | 1.2 | |
| | (MTHFR gene) | inflammatory processes which result in skin inflammation. | | | | |
| | rs610604 | Altered keratinocyte protein function resulting in | 1.0 | 1.1 | 1.2 | |
| | (TNFAIP3 gene) | exaggerated cytokine and chemokine production which can | | | | |
| | | lead to skin inflammation. | | | | |
| | rs17728338 | Loss of keratinocyte protein resulting in exaggerated | 1.0 | 1.1 | 1.2 | |
| | (TNIP1 gene) | cytokine and chemokine production which can lead to skin | | | | |
| | | inflammation. | | | | |
| | rs1800795 | Elevated IL-6 levels causing epidermal keratinocyte | 1.0 | 1.1 | 1.2 | |
| | (IL-6 gene) | hyperplasia (increase in the number of normal cells) | | | | |
| | | associated with skin inflammation. | | | | |
| Melanocyte | rs4646994 | Increased levels of free radicals, nitric oxide and IL-6 | 1.0 | 1.1 | 1.2 | rs4646994 |
| damage | (ACE gene) | leading to melanocyte damage | | | | |
| Collagen | rs1169732 | Disrupted collagen metabolism leading to skin aging | 1.0 | 1.1 | 1.2 | (rs1169732 + rs1799750) |
| degradation | (MMP9 gene) | | | | | 2 |
| | rs 1799750 | Disrupted collagen metabolism leading to skin aging | 1.0 | 1.1 | 1.2 | |
| | (MMP1 gene) | | | | | |
| Elastin | rs7787362 | Increased serum elastin levels leading to the risk of scar | 1.0 | 1.1 | 1.2 | rs7787362 |
| synthesis | (ELN1 gene) | formation | | | | |
| Antioxidant | rs1001179 | Lower antioxidant activity leading to antioxidant-mediated | 1.0 | 1.1 | 1.2 | |
| | (CAT gene) | skin damage. | | | | |
| | rs 1050450 | Poor antioxidant function leading to oxidative stress- | 1.0 | 1.1 | 1.2 | (rs1001179 + rs1050450 + |
| | (GPX1 gene) | mediated loss of melanocytes. | | | | rs1800566 + rs4880 + |
| | rs1800566 | Increased production of the free radical semiquinone | 1.0 | 1.1 | 1.2 | rs2234694) |
| | (NOQ1 gene) | leading to oxidative stress mediated-melanin synthesis. | | | | 5 |
| | rs4880 | Elevated hydrogen peroxide accumulation contributing to | 1.0 | 1.1 | 1.2 | |
| | (SOD2 gene) | oxidative stress-mediated loss of the epidermal | | | | |
| | | melanocytes. | | | | |
| | rs2234694 | Elevated hydrogen peroxide accumulation contributing to | 1.0 | 1.1 | 1.2 | |
| | (SOD2 gene) | oxidative stress-mediated loss of the epidermal | | | | |
| | | melanocytes. | | | | |
| Nutritional | rs7501331 | Alterations leading to a vitamin A deficiency which can | 1.0 | 1.1 | 1.2 | (rs7501331 + rs12934922) |
| needs | (BCMO1 gene) | accelerate the presence of wrinkles and ultimately skin | | | | 2 |
| | | aging. | | | | |
| | rs12934922 | Alterations leading to a vitamin A deficiency which can | 1.0 | 1.1 | 1.2 | |
| | (BCMO1) | accelerate the presence of wrinkles and ultimately skin | | | | |
| | | aging. | | | | |
| | rs4654748 | Mutation leads to a vitamin B6 deficiency which results in | 1.0 | 0.9 | 0.8 | rs4654748 |
| | (NBPF3 gene) | hormonal imbalance leading to skin inflammation and dry | | | | |
| | | skin. | | | | |
| | rs602662 | Mutation leads to a vitamin B12 deficiency which | 1.0 | 1.1 | 1.2 | rs602662 |
| | (FUT2 gene) | accelerates skin aging by increasing water loss, | | | | |
| | | inflammation, and redness. | | | | |
| | rs33972313 | Mutation leads to a vitamin C deficiency which increases | 1.0 | 1.1 | 1.2 | rs33972313 |
| | (SLC23A1 gene) | free radicals and oxidative stress in the skin leading to | | | | |
| | | oxidative stress-mediated skin damage. | | | | |
| | rs2282679 | Mutation leads to a vitamin D deficiency resulting in dry | 1.0 | 1.1 | 1.2 | rs2282679 |
| | (GC gene) | skin and Increased skin inflammation which may accelerate | | | | |
| | | skin aging. | | | | |
| | rs1801131 | Mutation leads to a folate deficiency resulting in poor skin | 1.0 | 1.1 | 1.2 | (rs1801131 + rs1801133) |
| | (MTHFR gene) | moisturization and elasticity which may accelerate skin | | | | 2 |
| | | aging. | | | | |
| | rs1801133 | Mutation leads to a folate deficiency resulting in poor skin | 1.0 | 1.1 | 1.2 | |
| | (MTHFR gene) | moisturization and elasticity which may accelerate skin | | | | |
| | | aging. | | | | |
| | rs 174547 | Mutation leads to an omega-3 fatty acid deficiency | 1.0 | 1.1 | 1.2 | rs174547 |
| | (FADS1 gene) | resulting in Increased skin inflammation. | | | | |

TABLE 5

| SNP Category | Topicals Suggestions |
|---|---|
| Melanin | 1. Hydroquinone (2-4%)-helps treat hyperpigmentation as it is a bleaching agent that acts by inhibiting tyrosinase to limit |
| synthesis | melanin production in the skin. |
| | 2. Retinoids-treat pigmentary conditions as they are effective in lightening skin by modulating cell proliferation and anti- |
| | inflammatory properties. |
| | 3. Azelaic acid-acts as a tyrosinase inhibitor as well as produces selective cytotoxic and antiproliferative effects on |
| | melanocytes, ultimately decreasing melanogenesis, thus preventing pigmentation. |
| | 4. Kojic acid-an effective depigmenting agent that works by inhibiting the activity of tyrosinase, thus preventing |
| | pigmentation. |
| | 5. Cysteamine-improves pigmentation by inhibiting tyrosinase and peroxidase, scavenging dopaquinone (melanin precursor), |
| | and chelating metal ions required for melanin synthesis. |
| | 6. Lignin peroxidase-acts by oxidizing and breaking down melanin, thereby exhibiting significant skin-lightening effects. |
| | 7. Niacinamide-combats hyperpigmentation by reversibly inhibiting the transfer of melanosomes to epidermal keratinocytes. |
| | 8. Ellagic acid-prevents pigmentation by inhibiting melanogenesis through the reduction of tyrosinase activity. |
| | 9. Arbutin-prevents pigmentation by reversing tyrosinase activity. |
| | 10. Vitamin C-promotes the differentiation of keratinocytes and decreases melanin (pigment) synthesis, leading to antioxidant |
| | protection against UV-induced photodamage, which helps in maintaining skin health. |
| Moisturization | 1. Vitamin E-an antioxidant and a very hydrating molecule since it works as both a humectant (to absorb water into the skin) |
| | and an emollient (to trap water in the skin). |
| | 2. Coenzyme Q10-the antioxidant protects the skin from environmental stressors, energizes the skin, and helps the skin retain |
| | moisture. |
| | 3. Vitamin F-has anti-inflammatory and moisturizing properties that help maintain the skin's barrier while relieving dryness |
| | and maintaining balance. |
| | 4. Kumazasa extract-increases AQP3 expression, which can transport water within the epidermis and help moisturize skin. |
| | 5. Magnesium ascorbyl phosphate-an antioxidant that easily absorbs into the skin and has a hydrating effect on the skin and |
| | decreases transepidermal water loss. |
| | 6. Hyaluronic acid-acts as a biological humectant (to absorb water into the skin) and helps in retaining water in the skin. |
| | 7. Magnesium oil-has absorption properties that make it a powerful humectant (to absorb water into the skin) to keep skin |
| | hydrated |
| Skin | 1. Coenzyme Q10-as anti-inflammatory properties, facilitating skin cell repair by lowering skin inflammatory mediators and |
| Inflammation | oxidative stress. |
| | 2. Methylsulfonylmethane-improve skin health by strengthening keratin and reducing skin inflammation. |
| | 3. Retinoid-reduces the formation of raised skin patches and the formation of cytokines and interleukins that cause skin |
| | inflammation. |
| | 4. Vitamin E-reduce UV-induced skin swelling, skin thickness, erythema, and edema-all signs of skin inflammation. |
| Melanocyte | Here, as skin inflammation and oxidative stress are causing melanocyte damage, the supplements suggested aim to reduce these root |
| damage | causes. |
| | 1. Coenzyme Q10-as anti-inflammatory properties, facilitating skin cell repair by lowering skin inflammatory mediators and |
| | oxidative stress. |
| | 2. Methylsulfonylmethane-improve skin health by strengthening keratin and reducing skin inflammation. |
| | 3. Retinoid-reduces the formation of raised skin patches and the formation of cytokines and interleukins that cause skin |
| | inflammation. |
| | 4. Vitamin E-reduce UV-induced skin swelling, skin thickness, erythema, and edema-all signs of skin inflammation. |
| Collagen | 1. Retinoids-help to reduce the levels of collagen inhibitors (MMPs and collagenases) and increase collagen synthesis. |
| degradation | 2. Vitamin C-vital antioxidant and an essential cofactor in collagen synthesis. |
| | 3. Vitamin E-a vital antioxidant that helps increase blood flow to the skin, which in turn helps produce collagen. |
| | 4. Tretinoin-shows prominent efficacy in regulating the proliferation and differentiation of epidermal cells and inducing new |
| | collagen formation. |
| Elastin | 1. Retinoid-improves skin elasticity by stimulating the production of collagen and by helping remove damaged elastin fibers, |
| synthesis | which might help with fading away stretch marks. |
| | 2. Vitamin C-plays a vital role in the production of collagen, giving skin its elasticity. This might be helpful in fading away |
| | the stretch marks. |
| | 3. Collagen-tightens loose skin, which helps in improving the elasticity of the skin and reducing the appearance of stretch |
| | marks. |
| | 4. Hyaluronic acid-firms out the contours of the skin and reduces the appearance of stretch marks. |
| | 5. Dexpanthenol-acts like a moisturizer, improving stratum corneum hydration, reducing transepidermal water loss, and |
| | maintaining skin softness and elasticity. This helps in reducing the appearance of stretch marks. |
| Antioxidant | 1. Vitamin C-protects the skin from oxidative stress by sequentially donating electrons to neutralize free radicals. |
| | 2. Coenzyme Q10-improves the antioxidant properties of stressed skin by free radical scavenging activity. |
| | 3. Polyphenols-can reduce inflammation, oxidative stress, and DNA-damaging effects of UV radiation in the skin. |
| | 4. Vitamin E-protects the skin from various deleterious effects due to solar radiation by acting as a free-radical scavenger. |
| Vitamin A | As a vitamin A deficiency can cause wrinkles and can accelerate aging, the following set of supplements aims to improve the same. |
| deficiency | 1. Retinol-promotes clear pores, increases collagen, and reduces fine lines, wrinkles, and skin discolorations. |
| | 2. Vitamin E-protects the skin from wrinkles and fine lines but can also boost collagen production, speeding up cell |
| | regeneration and creating new skin growth. |
| | 3. Resveratrol-improves fine lines, wrinkles, and elasticity by neutralizing free radicals and boosting antioxidant levels, so |
| | that the skin can better defend and repair itself. |
| | 4. Baicalin-absorbs UV rays, has antioxidant and anti-inflammatory properties; reduces the skin's oxidative stress and skin |
| | inflammation that may delay the photoaging process. |
| | 5. B-vitamins-improve the skin's texture and moisture retention. It reduces the appearance of fine lines and wrinkles because |
| | it helps the skin stay moist (wrinkles and lines are exaggerated with dry skin). |
| | 6. Vitamin C-boosts collagen production, resulting in an overall tightening effect. |
| Vitamin B6 | The supplements suggested below help with improving skin inflammation and dry skin which can be caused due to a vitamin B6 |
| deficiency | deficiency. |
| | 1. B-vitamins-can act as an antioxidant that helps reduce the signs of skin inflammation such as rash, redness, and irritation. |
| | 2. Coenzyme Q10-as anti-inflammatory properties, facilitating skin cell repair by lowering skin inflammatory mediators and |
| | oxidative stress. |
| | 3. Methylsulfonylmethane-improve skin health by strengthening keratin and reducing skin inflammation. |
| | 4. Retinoid-reduces the formation of raised skin patches and the formation of cytokines and interleukins that cause skin |
| | inflammation. |
| | 5. Vitamin E-reduce UV-induced skin swelling, skin thickness, erythema, and edema-all signs of skin inflammation. |
| Vitamin B12 | As a vitamin B12 deficiency can accelerate skin aging, the following set of supplements aim to improve the same. |
| deficiency | 1. Retinol-promotes clear pores, increases collagen, and reduces fine lines, wrinkles, and skin discolorations. |
| | 2. Vitamin E-protects the skin from wrinkles and fine lines but can also boost collagen production, speeding up cell |
| | regeneration and creating new skin growth. |
| | 3. Resveratrol-improves fine lines, wrinkles, and elasticity by neutralizing free radicals and boosting antioxidant levels, so |
| | that the skin can better defend and repair itself. |
| | 4. Baicalin-absorbs UV rays, has antioxidant and anti-inflammatory properties; reduces the skin's oxidative stress and skin |
| | inflammation that may delay the photoaging process. |
| | 5. B-vitamins-improve the skin's texture and moisture retention. It reduces the appearance of fine lines and wrinkles because |
| | it helps the skin stay moist (wrinkles and lines are exaggerated with dry skin). |
| | 6. Vitamin C-boosts collagen production, resulting in an overall tightening effect. |
| Vitamin C | The supplements suggested below help with improving skin inflammation and oxidative stress which can be brought about by a |
| deficiency | vitamin C deficiency. |
| | 1. Vitamin C-protects the skin from oxidative stress by sequentially donating electrons to neutralize free radicals. |
| | 2. Coenzyme Q10-improves the antioxidant properties of stressed skin by free radical scavenging activity. |
| | 3. Polyphenols-can reduce inflammation, oxidative stress, and DNA-damaging effects of UV radiation in the skin. |
| | 4. Vitamin E-protects the skin from various deleterious effects due to solar radiation by acting as a free-radical scavenger. |
| Vitamin D | As a vitamin D deficiency can skin inflammation and can accelerate skin aging, the following set of supplements aims to improve the |
| deficiency | same. |
| | 1. Vitamin D-reduces oxidative stress and signs of skin inflammation such as rash, redness, and irritation by acting as an |
| | antioxidant; and prevents premature aging. |
| | 2. Retinol-promotes clear pores, increases collagen, and reduces fine lines, wrinkles, and skin discolorations. |
| | 3. Vitamin E-protects the skin from wrinkles and fine lines but can also boost collagen production, speeding up cell |
| | regeneration and creating new skin growth. |
| | 4. Resveratrol-improves fine lines, wrinkles, and elasticity by neutralizing free radicals and boosting antioxidant levels, so |
| | that the skin can better defend and repair itself. |
| | 5. Baicalin-absorbs UV rays, has antioxidant and anti-inflammatory properties; reduces the skin's oxidative stress and skin |
| | inflammation that may delay the photoaging process. |
| | 6. B-vitamins-improve the skin's texture and moisture retention. It reduces the appearance of fine lines and wrinkles because |
| | it helps the skin stay moist (wrinkles and lines are exaggerated with dry skin). |
| | 7. Vitamin C-boosts collagen production, resulting in an overall tightening effect. |
| Folate | As folate deficiency can affect the skin's moisture content and can accelerate skin aging, the following set of supplements aims to |
| deficiency | improve the factors. |
| | 1. Folate-alleviates dry skin by maintaining skin barrier function via moisture retention; and reduces the signs of premature |
| | aging by maintaining adequate collagen production. |
| | 2. Retinol-promotes clear pores, increases collagen, and reduces fine lines, wrinkles, and skin discolorations. |
| | 3. Vitamin E-protects the skin from wrinkles and fine lines but can also boost collagen production, speeding up cell |
| | regeneration and creating new skin growth. |
| | 4. Resveratrol-improves fine lines, wrinkles, and elasticity by neutralizing free radicals and boosting antioxidant levels, so |
| | that the skin can better defend and repair itself. |
| | 5. Baicalin-absorbs UV rays, has antioxidant and anti-inflammatory properties; reduces the skin's oxidative stress and skin |
| | inflammation that may delay the photoaging process. |
| | 6. B-vitamins-improve the skin's texture and moisture retention. It reduces the appearance of fine lines and wrinkles because |
| | it helps the skin stay moist (wrinkles and lines are exaggerated with dry skin). |
| | 7. Vitamin C-boosts collagen production, resulting in an overall tightening effect. |
| Omega fatty | The following set of supplements aim at reducing skin inflammation which is associated with an omega-fatty acid deficiency. |
| acid | 1. DHA (Docosahexaenoic acid)-reduces the signs of skin inflammation such as rash, redness, and irritation by upregulating |
| deficiency | antioxidative protein markers Nrf2 and HO-1; controls aging by protecting skin from damage. |
| | 2. Coenzyme Q10-as anti-inflammatory properties, facilitating skin cell repair by lowering skin inflammatory mediators and |
| | oxidative stress. |
| | 3. Methylsulfonylmethane-improve skin health by strengthening keratin and reducing skin inflammation. |
| | 4. Retinoid-reduces the formation of raised skin patches and the formation of cytokines and interleukins that cause skin |
| | inflammation. |
| | 5. Vitamin E-reduce UV-induced skin swelling, skin thickness, erythema, and edema-all signs of skin inflammation. |

TABLE 6

| SNP Category | rs ID | Outcome | Supplement Suggestions |
|---|---|---|---|
| Melanin | rs1426654 | Differences in melanin | 1. Vitamin C-promotes the differentiation of keratinocytes and |
| synthesis | (SLC24A5 | production leading to | decreases melanin; |
| | gene) | pigmentation | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs16891982 | Accumulation of melanin- | 1. Vitamin C-promotes the differentiation of keratinocytes and |
| | (SLC45A2 | containing melanosomes in the | decreases melanin; |
| | gene) | epidermis leading to | 2. L-Cystine-decreases tyrosinase activity; |
| | | pigmentation | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs1042602 | Accumulation of melanin- | 1. Vitamin C-promotes the differentiation of keratinocytes and |
| | (TYR gene) | containing melanosomes in the | decreases melanin; |
| | | epidermis leading to | 2. L-Cystine-decreases tyrosinase activity; |
| | | pigmentation | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs1393350 | Accumulation of melanin- | 1. Vitamin C-promotes the differentiation of keratinocytes and |
| | (TYR gene) | containing melanosomes in the | decreases melanin; |
| | | epidermis leading to | 2. L-Cystine-decreases tyrosinase activity; |
| | | pigmentation | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs6058017 | Alteration in the gene regulating | 1. Vitamin C-promotes the differentiation of keratinocytes and |
| | (ASIP gene) | melanogenesis leading to | decreases melanin; |
| | | pigmentation | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs74653330 | Alteration in the gene regulating | 1. Vitamin C-promotes the differentiation of keratinocytes and |
| | (OCA2 gene) | melanogenesis leading to | decreases melanin; |
| | | pigmentation | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs1393350 | Altered skin sensitivity to the | 1. Vitamin C-promotes the differentiation of keratinocytes and |
| | (TYR gene) | sun leading to skin burn and | decreases melanin; |
| | | freckles | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs1805008 | Overproduction of melanin | 1. Vitamin C-promotes the differentiation of keratinocytes and |
| | (MC1R gene) | leading to pigmentation | decreases melanin; |
| | | | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs1015362 | Altered skin sensitivity to the | 1. Vitamin C-promotes the differentiation of keratinocytes and |
| | (ASIP gene) | sun leading to skin burns and | decreases melanin; |
| | | freckles | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs4911414 | Altered skin sensitivity to the | 1. Vitamin C-promotes the differentiation of keratinocytes and |
| | (ASIP gene) | sun leading to skin burns and | decreases melanin; |
| | | freckles | 2. L-Cystine-decreases tyrosinase activity; |
| | | | 3. L-Glutathione-decreases tyrosinase activity; |
| | | | 4. Polypodium leucotomos-improves pigmentation |
| | | | 5. Melatonin-inhibits α-MSH receptors and reduces pigmentation |
| | rs12203592 | Poor activation of TYR enzyme | Mutant has a lighter skin phenotype |
| | (IRF4 gene) | required for melanin synthesis | |
| | rs1805007 | Increased production of | Homozygous mutant individuals have light skin and poor tanning ability. |
| | (MC1R gene) | phaeomelanin, resulting in light | |
| | | skin and poor tanning ability | |
| | rs1805009 | Increased production of | Homozygous mutant individuals have light skin and poor tanning ability. |
| | (MC1R gene) | phaeomelanin, resulting in light | |
| | | skin and poor tanning ability | |
| Moisturization | rs17553719 | Increased water loss from the | 1. Collagen-increases the skin's moisturizing capacity and reduces trans |
| | (AQP3 gene) | skin leading to reduced skin | epidermal water loss; |
| | | moisturization capacity | 2. Ceramides-improve the skin's moisture content by stimulating |
| | | | collagen synthesis and elevating the expression of aquaporin-3; |
| | | | 3. Hyaluronan-inhibits the skin's moisture loss; |
| | | | 4. Procyanidin-improves the skin's moisture content by increasing the |
| | | | expression of aquaporin 3; |
| | | | 5. Probiotics-may improve moisture content by improving |
| | | | transepidermal water loss, and producing skin barrier proteins; |
| | | | 6. Galactooligosaccharides-improves trans epidermal water loss; |
| | | | 7. Alpha-linolenic acid-improves trans epidermal water loss, skin |
| | | | scaling and roughness, and skin hydration; |
| | | | 8. Gamma-linolenic acid-increases ceramide synthesis and improves |
| | | | skin barrier function which helps retain skin moisture. |
| | rs61816761 | Shortage of natural moisturizing | 1. Collagen-increases the skin's moisturizing capacity and reduces trans |
| | (FLG gene) | factors and impaired skin barrier | epidermal water loss; |
| | | function affecting skin | 2. Ceramides-improve the skin's moisture content by stimulating |
| | | moisturization capacity | collagen synthesis and elevating the expression of aquaporin-3; |
| | | | 3. Hyaluronan-inhibits the skin's moisture loss; |
| | | | 4. Procyanidin-improves the skin's moisture content by increasing the |
| | | | expression of aquaporin 3; |
| | | | 5. Probiotics-may improve moisture content by improving |
| | | | transepidermal water loss, and producing skin barrier proteins; |
| | | | 6. Galactooligosaccharides-improves trans epidermal water loss; |
| | | | 7. Alpha-linolenic acid-improves trans epidermal water loss, skin |
| | | | scaling and roughness, and skin hydration; |
| | | | 8. Gamma-linolenic acid-increases ceramide synthesis and improves |
| | | | skin barrier function which helps retain skin moisture. |
| | rs138726443 | Shortage of natural moisturizing | 1. Collagen-increases the skin's moisturizing capacity and reduces trans |
| | (FLG gene) | factors and impaired skin barrier | epidermal water loss; |
| | | function affecting skin | 2. Ceramides-improves the skin's moisture content by stimulating |
| | | moisturization capacity | collagen synthesis and elevating the expression of aquaporin-3; |
| | | | 3. Hyaluronan-inhibits the skin's moisture loss; |
| | | | 4. Procyanidin-improves the skin's moisture content by increasing the |
| | | | expression of aquaporin 3; |
| | | | 5. Probiotics-may improve moisture content by improving |
| | | | transepidermal water loss, and producing skin barrier proteins; |
| | | | 6. Galactooligosaccharides-improves trans epidermal water loss; |
| | | | 7. Alpha-linolenic acid-improves trans epidermal water loss, skin |
| | | | scaling and roughness, and skin hydration; |
| | | | 8. Gamma-linolenic acid-increases ceramide synthesis and improves |
| | | | skin barrier function which helps retain skin moisture. |
| | rs 150597413 | Shortage of natural moisturizing | 1. Collagen-increases the skin's moisturizing capacity and reduces trans |
| | (FLG gene) | factors and impaired skin barrier | epidermal water loss; |
| | | function affecting skin | 2. Ceramides-improves the skin's moisture content by stimulating |
| | | moisturization capacity | collagen synthesis and elevating the expression of aquaporin-3; |
| | | | 3. Hyaluronan-inhibits the skin's moisture loss; |
| | | | 4. Procyanidin-improves the skin's moisture content by increasing the |
| | | | expression of aquaporin 3; |
| | | | 5. Probiotics-may improve moisture content by improving |
| | | | transepidermal water loss, and producing skin barrier proteins; |
| | | | 6. Galactooligosaccharides-improves trans epidermal water loss; |
| | | | 7. Alpha-linolenic acid-improves trans epidermal water loss, skin |
| | | | scaling and roughness, and skin hydration; |
| | | | 8. Gamma-linolenic acid-increases ceramide synthesis and improves |
| | | | skin barrier function which helps retain skin moisture. |
| Skin | rs20541 | Elevated IL-13 levels leading to | 1. Vitamin C-helps reduce the signs of skin inflammation such as rash, |
| Inflammation | (IL-13 gene) | skin inflammation and lesions | redness, and irritation owing to its antioxidant and anti-inflammatory |
| | | | functions; |
| | | | 2. Curcumin-protects the skin by quenching free radicals and reduces |
| | | | skin inflammation; |
| | | | 3. Green tea-reduces skin inflammation owing to its antioxidant and |
| | | | anti-inflammatory functions; |
| | | | 4. Omega 3 fatty acids-helps reduce rash, redness, and irritation owing |
| | | | to its antioxidant and ant inflammatory functions; |
| | | | 5. Vitamin E-is a strong antioxidant preventing skin inflammation |
| | | | caused due to lipid peroxide irritation. |
| | rs2201841 | Elevated IL-23 levels leading to | 1. Vitamin C-helps reduce the signs of skin inflammation such as rash, |
| | (IL23R gene) | skin inflammation and lesions | redness, and irritation owing to its antioxidant and anti-inflammatory |
| | | | functions; |
| | | | 2. Curcumin-protects the skin by quenching free radicals and reduces |
| | | | skin inflammation; |
| | | | 3. Green tea-reduces skin inflammation owing to its antioxidant and |
| | | | anti-inflammatory functions; |
| | | | 4. Omega 3 fatty acids-helps reduce rash, redness, and irritation owing |
| | | | to its antioxidant and ant inflammatory functions; |
| | | | 5. Vitamin E-is a strong antioxidant preventing skin inflammation |
| | | | caused due to lipid peroxide irritation. |
| | rs1801133 | Elevated homocysteine levels | 1. Vitamin C-helps reduce the signs of skin inflammation such as rash, |
| | (MTHFR gene) | leading to immuno- | redness, and irritation owing to its antioxidant and anti-inflammatory |
| | | inflammatory processes which | functions; |
| | | result in skin inflammation. | 2. Curcumin-protects the skin by quenching free radicals and reduces |
| | | | skin inflammation; |
| | | | 3. Green tea-reduces skin inflammation owing to its antioxidant and |
| | | | anti-inflammatory functions; |
| | | | 4. Omega 3 fatty acids-helps reduce rash, redness, and irritation owing |
| | | | to its antioxidant and ant inflammatory functions; |
| | | | 5. Vitamin E-is a strong antioxidant preventing skin inflammation |
| | | | caused due to lipid peroxide irritation. |
| | rs610604 | Altered keratinocyte protein | 1. Vitamin C-helps reduce the signs of skin inflammation such as rash, |
| | (TNFAIP3 | function resulting in exaggerated | redness, and irritation owing to its antioxidant and anti-inflammatory |
| | gene) | cytokine and chemokine | functions; |
| | | production which can lead to | 2. Curcumin-protects the skin by quenching free radicals and reduces |
| | | skin inflammation. | skin inflammation; |
| | | | 3. Green tea-reduces skin inflammation owing to its antioxidant and |
| | | | anti-inflammatory functions; |
| | | | 4. Omega 3 fatty acids-helps reduce rash, redness, and irritation owing |
| | | | to its antioxidant and ant inflammatory functions; |
| | | | 5. Vitamin E-is a strong antioxidant preventing skin inflammation |
| | | | caused due to lipid peroxide irritation. |
| | rs17728338 | Loss of keratinocyte protein | 1. Vitamin C-helps reduce the signs of skin inflammation such as rash, |
| | (TNIP1 gene) | resulting in exaggerated cytokine | redness, and irritation owing to its antioxidant and anti-inflammatory |
| | | and chemokine production | functions; |
| | | which can lead to skin | 2. Curcumin-protects the skin by quenching free radicals and reduces |
| | | inflammation. | skin inflammation; |
| | | | 3. Green tea-reduces skin inflammation owing to its antioxidant and |
| | | | anti-inflammatory functions; |
| | | | 4. Omega 3 fatty acids-helps reduce rash, redness, and irritation owing |
| | | | to its antioxidant and ant inflammatory functions; |
| | | | 5. Vitamin E-is a strong antioxidant preventing skin inflammation |
| | | | caused due to lipid peroxide irritation. |
| | rs1800795 | Elevated IL-6 levels causing | 1. Vitamin C-helps reduce the signs of skin inflammation such as rash, |
| | (IL-6 gene) | epidermal keratinocyte | redness, and irritation owing to its antioxidant and anti-inflammatory |
| | | hyperplasia (increase in the | functions; |
| | | number of normal cells) | 2. Curcumin-protects the skin by quenching free radicals and reduces |
| | | associated with skin | skin inflammation; |
| | | inflammation. | 3. Green tea-reduces skin inflammation owing to its antioxidant and |
| | | | anti-inflammatory functions; |
| | | | 4. Omega 3 fatty acids-helps reduce rash, redness, and irritation owing |
| | | | to its antioxidant and ant inflammatory functions; |
| | | | 5. Vitamin E-is a strong antioxidant preventing skin inflammation |
| | | | caused due to lipid peroxide irritation. |
| Melanocyte | rs4646994 | Increased levels of free radicals, | 1. Vitamin D-decreases the expression of cytokines that cause skin |
| damage | (ACE gene) | nitric oxide, and IL-6 leading to | inflammation that might help with preventing the destruction of |
| | | melanocyte damage | melanocytes; |
| | | | 2. Zinc-protects melanocyte damage via antioxidant properties and anti- |
| | | | inflammatory activity; |
| | | | 3. Vitamin C-has antioxidant functions that reduces oxidative stress; |
| | | | 4. Curcumin-quenches free radicals and reduces skin oxidative stress; |
| | | | 5. Green tea-reduces skin inflammation owing to its antioxidant and |
| | | | anti-inflammatory functions; |
| | | | 6. Omega 3 fatty acids-helps reduce rash, redness, and irritation owing |
| | | | to its antioxidant and ant inflammatory functions; |
| | | | 7. Vitamin E-is a strong antioxidant preventing skin inflammation |
| | | | caused due to lipid peroxide irritation. |
| Collagen | rs1169732 | Disrupted collagen metabolism | 1. Collagen peptides-increases collagen content, improves the |
| degradation | (MMP9 gene) | leading to skin aging | elasticity of the skin, and reduces wrinkling and roughness; |
| | | | 2. Vitamin C-aids in the production of collagen; |
| | | | 3. Red ginseng-increases type 1 procollagen synthesis; |
| | | | 4. Aloe vera extract-increases collagen production and decreases |
| | | | collagen-degrading MMP-1 gene expression; |
| | | | 5. Retinol-inhibits UV induction of MMP and stimulates collagen |
| | | | synthesis; |
| | | | 6. Hyaluronic acid-aids in the production of collagen; |
| | | | 7. Ginkgo biloba-increases the proliferation of skin fibroblasts and |
| | | | enhances the production of collagen; |
| | | | 8. Soy-prevents the activation of matrix degradation enzymes and |
| | | | enhances the production of collagen; |
| | | | 9. Green tea extracts-prevents the activation of matrix degradation |
| | | | enzymes and enhances the production of collagen. |
| | rs 1799750 | Disrupted collagen metabolism | 1. Collagen peptides-increases collagen content, improves the |
| | (MMP1 gene) | leading to skin aging | elasticity of the skin, and reduces wrinkling and roughness; |
| | | | 2. Vitamin C-aids in the production of collagen; |
| | | | 3. Red ginseng-increases type 1 procollagen synthesis; |
| | | | 4. Aloe vera extract-increases collagen production and decreases |
| | | | collagen-degrading MMP-1 gene expression; |
| | | | 5. Retinol-inhibits UV induction of MMP and stimulates collagen |
| | | | synthesis; |
| | | | 6. Hyaluronic acid-aids in the production of collagen; |
| | | | 7. Ginkgo biloba-increases the proliferation of skin fibroblasts and |
| | | | enhances the production of collagen; |
| | | | 8. Soy-prevents the activation of matrix degradation enzymes and |
| | | | enhances the production of collagen; |
| | | | 9. Green tea extracts-prevents the activation of matrix degradation |
| | | | enzymes and enhances the production of collagen. |
| Elastin | rs7787362 | Increased serum elastin levels | 1. Collagen peptides-improves the skin's elasticity and reduces scar |
| synthesis | (ELN1 gene) | leading to the risk of scar | formation; |
| | | formation | 2. Vitamin C-boosts collagen synthesis which will help with |
| | | | improving skin elasticity and scar reduction; |
| | | | 3. Vitamin E-boosts collagen synthesis which will help with improving |
| | | | skin elasticity and scar reduction; |
| | | | 4. Vitamin A-improves skin elasticity and skin sagging by helping |
| | | | remove damaged elastin fibers; |
| | | | 5. Zinc-promotes collagen synthesis and restores functional elastin |
| | | | which aids in healing scars. |
| Antioxidant | rs1001179 | Lower antioxidant activity | 1. Curcumin-quenches free radicals and reduces oxidative stress; |
| | (CAT gene) | leading to antioxidant-mediated | 2. Vitamin C-acts as a crucial antioxidant which prevents the skin from |
| | | skin damage. | oxidative damage; |
| | | | 3. Vitamin E-acts as a crucial antioxidant which prevents the skin from |
| | | | oxidative damage; |
| | | | 4. Beta carotene-acts as a crucial antioxidant which prevents the skin |
| | | | from oxidative damage; |
| | | | 5. Flavanols-possess antioxidant activity that prevent the skin from |
| | | | oxidative damage; |
| | | | 6. Zinc-scavenges free radicals and prevents the skin from oxidative |
| | | | damage; |
| | | | 7. Lutein-scavenges free radicals and prevents the skin from oxidative |
| | | | damage; |
| | | | 8. Lycopene-scavenges free radicals and prevents the skin from |
| | | | oxidative damage; |
| | | | 9. Silymarin-scavenges free radicals and prevents the skin from |
| | | | oxidative damage; |
| | | | 10. Green tea-eliminates free radicals and prevents the skin from |
| | | | oxidative damage. |
| | rs 1050450 | Poor antioxidant function | 1. Curcumin-quenches free radicals and reduces oxidative stress; |
| | (GPX1 gene) | leading to oxidative stress- | 2. Vitamin C-acts as a crucial antioxidant which prevents the skin from |
| | | mediated loss of melanocytes. | oxidative damage; |
| | | | 3. Vitamin E-acts as a crucial antioxidant which prevents the skin from |
| | | | oxidative damage; |
| | | | 4. Beta carotene-acts as a crucial antioxidant which prevents the skin |
| | | | from oxidative damage; |
| | | | 5. Flavanols-possess antioxidant activity that prevent the skin from |
| | | | oxidative damage; |
| | | | 6. Zinc-scavenges free radicals and prevents the skin from oxidative |
| | | | damage; |
| | | | 7. Lutein-scavenges free radicals and prevents the skin from oxidative |
| | | | damage; |
| | | | 8. Lycopene-scavenges free radicals and prevents the skin from |
| | | | oxidative damage; |
| | | | 9. Silymarin-scavenges free radicals and prevents the skin from |
| | | | oxidative damage; |
| | | | 10. Green tea-eliminates free radicals and prevents the skin from |
| | | | oxidative damage. |
| | rs1800566 | Increased production of the free | 1. Curcumin-quenches free radicals and reduces oxidative stress; |
| | (NOQ1 gene) | radical semiquinone leading to | 2. Vitamin C-acts as a crucial antioxidant which prevents the skin from |
| | | oxidative stress mediated- | oxidative damage; |
| | | melanin synthesis. | 3. Vitamin E-acts as a crucial antioxidant which prevents the skin from |
| | | | oxidative damage; |
| | | | 4. Beta carotene-acts as a crucial antioxidant which prevents the skin |
| | | | from oxidative damage; |
| | | | 5. Flavanols-possess antioxidant activity that prevent the skin from |
| | | | oxidative damage; |
| | | | 6. Zinc-scavenges free radicals and prevents the skin from oxidative |
| | | | damage; |
| | | | 7. Lutein-scavenges free radicals and prevents the skin from oxidative |
| | | | damage; |
| | | | 8. Lycopene-scavenges free radicals and prevents the skin from |
| | | | oxidative damage; |
| | | | 9. Silymarin-scavenges free radicals and prevents the skin from |
| | | | oxidative damage; |
| | | | 10. Green tea-eliminates free radicals and prevents the skin from |
| | | | oxidative damage. |
| | rs4880 | Elevated hydrogen peroxide | 1. Curcumin-quenches free radicals and reduces oxidative stress; |
| | (SOD2 gene) | accumulation contributing to | 2. Vitamin C-acts as a crucial antioxidant which prevents the skin from |
| | | oxidative stress-mediated loss of | oxidative damage; |
| | | the epidermal melanocytes. | 3. Vitamin E-acts as a crucial antioxidant which prevents the skin from |
| | | | oxidative damage; |
| | | | 4. Beta carotene-acts as a crucial antioxidant which prevents the skin |
| | | | from oxidative damage; |
| | | | 5. Flavanols-possess antioxidant activity that prevent the skin from |
| | | | oxidative damage; |
| | | | 6. Zinc-scavenges free radicals and prevents the skin from oxidative |
| | | | damage; |
| | | | 7. Lutein-scavenges free radicals and prevents the skin from oxidative |
| | | | damage; |
| | | | 8. Lycopene-scavenges free radicals and prevents the skin from |
| | | | oxidative damage; |
| | | | 9. Silymarin-scavenges free radicals and prevents the skin from |
| | | | oxidative damage; |
| | | | 10. Green tea-eliminates free radicals and prevents the skin from |
| | | | oxidative damage. |
| | rs2234694 | Elevated hydrogen peroxide | 1. Curcumin-quenches free radicals and reduces oxidative stress; |
| | (SOD2 gene) | accumulation contributing to | 2. Vitamin C-acts as a crucial antioxidant which prevents the skin from |
| | | oxidative stress-mediated loss of | oxidative damage; |
| | | the epidermal melanocytes. | 3. Vitamin E-acts as a crucial antioxidant which prevents the skin from |
| | | | oxidative damage; |
| | | | 4. Beta carotene-acts as a crucial antioxidant which prevents the skin |
| | | | from oxidative damage; |
| | | | 5. Flavanols-possess antioxidant activity that prevent the skin from |
| | | | oxidative damage; |
| | | | 6. Zinc-scavenges free radicals and prevents the skin from oxidative |
| | | | damage; |
| | | | 7. Lutein-scavenges free radicals and prevents the skin from oxidative |
| | | | damage; |
| | | | 8. Lycopene-scavenges free radicals and prevents the skin from |
| | | | oxidative damage; |
| | | | 9. Silymarin-scavenges free radicals and prevents the skin from |
| | | | oxidative damage; |
| | | | 10. Green tea-eliminates free radicals and prevents the skin from |
| | | | oxidative damage. |
| Nutritional | rs7501331 | Alterations leading to a vitamin | 1. Vitamin A-improves vitamin A deficiency and stimulates collagen |
| needs | (BCMO1 gene) | A deficiency which can | production which aids in reducing wrinkles and fine lines; |
| | | accelerate the presence of | 2. Epigallocatechin gallate-helps fight wrinkles and rejuvenate skin |
| | | wrinkles and ultimately skin | cells; |
| | | aging. | 3. Collagen-helps decelerate skin aging by reducing wrinkling and |
| | | | roughness; |
| | | | 4. Nicotinamide riboside-helps restore NAD+ levels which have been |
| | | | shown to reverse key indicators of aging by reducing wrinkles, improving |
| | | | moisture retention, and smoothening skin texture; |
| | | | 5. Nicotinamide mononucleotide-increases NAD+ levels which have |
| | | | been shown to reverse key indicators of aging by reducing wrinkles, |
| | | | improving moisture retention, and smoothening skin texture; |
| | | | 6. Coenzyme Q10-increases elastin production that tightens the skin, |
| | | | improves elasticity, and reduces fine lines and wrinkles; |
| | | | 7. Vitamin C-boosts collagen synthesis which helps reduce fine lines |
| | | | and wrinkles; |
| | | | 8. Curcumin-quenches free radicals and reduces oxidative stress which |
| | | | helps control aging by protecting the skin from oxidative damage. |
| | rs12934922 | Alterations leading to a vitamin | 1. Vitamin A-improves vitamin A deficiency and stimulates collagen |
| | (BCMO1) | A deficiency which can | production which aids in reducing wrinkles and fine lines; |
| | | accelerate the presence of | 2. Epigallocatechin gallate-helps fight wrinkles and rejuvenate skin |
| | | wrinkles and ultimately skin | cells; |
| | | aging. | 3. Collagen-helps decelerate skin aging by reducing wrinkling and |
| | | | roughness; |
| | | | 4. Nicotinamide riboside-helps restore NAD+ levels which have been |
| | | | shown to reverse key indicators of aging by reducing wrinkles, improving |
| | | | moisture retention, and smoothening skin texture; |
| | | | 5. Nicotinamide mononucleotide-increases NAD+ levels which have |
| | | | been shown to reverse key indicators of aging by reducing wrinkles, |
| | | | improving moisture retention, and smoothening skin texture; |
| | | | 6. Coenzyme Q10-increases elastin production that tightens the skin, |
| | | | improves elasticity, and reduces fine lines and wrinkles; |
| | | | 7. Vitamin C-boosts collagen synthesis which helps reduce fine lines |
| | | | and wrinkles; |
| | | | 8. Curcumin-quenches free radicals and reduces oxidative stress which |
| | | | helps control aging by protecting the skin from oxidative damage. |
| | rs4654748 | Mutation leads to a vitamin B6 | 1. Vitamin B6-improves vitamin B6 deficiency and helps reduce the |
| | (NBPF3 gene) | deficiency which results in | signs of skin inflammation such as rash, redness, and irritation; |
| | | hormonal imbalance leading to | 2. Curcumin-quenches free radicals and reduces oxidative stress which |
| | | skin inflammation and dry skin. | helps control aging by protecting the skin from oxidative damage; |
| | | | 3. Green tea extract-reduces skin inflammation and protects the skin |
| | | | from damage; |
| | | | 4. Omega 3 fatty acids-reduces the signs of skin inflammation such as |
| | | | rash, redness, and irritation; |
| | | | 5. Vitamin E-is a strong antioxidant preventing skin inflammation |
| | | | caused due to lipid peroxide irritation. |
| | | | 6. Vitamin C-reduces the signs of skin inflammation such as rash, |
| | | | redness, and irritation. |
| | rs602662 | Mutation leads to a vitamin B12 | 1. Vitamin B12-improves vitamin B12 deficiency and aids in reducing |
| | (FUT2 gene) | deficiency which accelerates | wrinkles and fine lines; |
| | | skin aging by increasing water | 2. Curcumin-quenches free radicals and reduces oxidative stress which |
| | | loss, inflammation, and redness. | helps control aging by protecting the skin from oxidative damage; |
| | | | 3. Epigallocatechin gallate -slows down the process of skin aging by |
| | | | reducing wrinkles, improving moisture retention, and smoothening skin |
| | | | texture; |
| | | | 4. Collagen-helps decelerate skin aging by reducing wrinkling and |
| | | | roughness; |
| | | | 5. Nicotinamide riboside-helps restore NAD+ levels which have been |
| | | | shown to reverse key indicators of aging by reducing wrinkles, improving |
| | | | moisture retention, and smoothening skin texture; |
| | | | 6. Nicotinamide mononucleotide-increases NAD+ levels which have |
| | | | been shown to reverse key indicators of aging by reducing wrinkles, |
| | | | improving moisture retention, and smoothening skin texture; |
| | | | 7. Coenzyme Q10-increases elastin production that tightens the skin, |
| | | | improves elasticity, and reduces fine lines and wrinkles; |
| | | | 8. Vitamin C-boosts collagen synthesis which helps reduce fine lines |
| | | | and wrinkles. |
| | rs33972313 | Mutation leads to a vitamin C | 1. Vitamin C-improves vitamin C deficiency and acts as a crucial |
| | (SLC23A1 gene) | deficiency which increases free | antioxidant that prevents the skin from oxidative damage; |
| | | radicals and oxidative stress in | 2. Curcumin-quenches free radicals and reduces oxidative stress; |
| | | the skin leading to oxidative | 3. Vitamin E-acts as a crucial antioxidant which prevents the skin from |
| | | stress-mediated skin damage. | oxidative damage; |
| | | | 4. Beta carotene-acts as a crucial antioxidant which prevents the skin |
| | | | from oxidative damage; |
| | | | 5. Flavanols-possess antioxidant activity that prevent the skin from |
| | | | oxidative damage; |
| | | | 6. Zinc-scavenges free radicals and prevents the skin from oxidative |
| | | | damage; |
| | | | 7. Lutein-scavenges free radicals and prevents the skin from oxidative |
| | | | damage; |
| | | | 8. Lycopene-scavenges free radicals and prevents the skin from |
| | | | oxidative damage; |
| | | | 9. Silymarin-scavenges free radicals and prevents the skin from |
| | | | oxidative damage; |
| | | | 10. Green tea-eliminates free radicals and prevents the skin from |
| | | | oxidative damage. |
| | rs2282679 | Mutation leads to a vitamin D | 1. Vitamin D-improves vitamin D deficiency, reduces skin |
| | (GC gene) | deficiency resulting in dry skin | inflammation, scavenges free radicals, and prevents the skin from premature |
| | | and increased skin inflammation | aging; |
| | | which may accelerate skin aging. | 2. Curcumin-quenches free radicals, reduces oxidative stress, and |
| | | | prevents skin damage; |
| | | | 3. Epigallocatechin gallate-slows down the process of skin aging by |
| | | | reducing wrinkles, improving moisture retention, and smoothening skin |
| | | | texture; |
| | | | 4. Collagen-helps decelerate skin aging by reducing wrinkling and |
| | | | roughness; |
| | | | 5. Nicotinamide riboside-helps restore NAD+ levels which have been |
| | | | shown to reverse key indicators of aging by reducing wrinkles, improving |
| | | | moisture retention, and smoothening skin texture; |
| | | | 6. Nicotinamide mononucleotide-increases NAD+ levels which have |
| | | | been shown to reverse key indicators of aging by reducing wrinkles, |
| | | | improving moisture retention, and smoothening skin texture; |
| | | | 7. Coenzyme Q10-increases elastin production that tightens the skin, |
| | | | improves elasticity, and reduces fine lines and wrinkles; |
| | | | 8. Vitamin C-boosts collagen synthesis which helps reduce fine lines |
| | | | and wrinkles. |
| | rs1801131 | Mutation leads to a folate | 1. Folate-improves folate deficiency, maintains skin barrier function by |
| | (MTHFR gene) | deficiency resulting in poor skin | retaining moisture and alleviates skin dryness, prevents premature skin |
| | | moisturization and elasticity | aging by enhancing collagen production; |
| | | which may accelerate skin aging. | 2. Curcumin-quenches free radicals, reduces oxidative stress, and |
| | | | prevents skin damage; |
| | | | 3. Epigallocatechin gallate-slows down the process of skin aging by |
| | | | reducing wrinkles, improving moisture retention, and smoothening skin |
| | | | texture; |
| | | | 4. Collagen-helps decelerate skin aging by reducing wrinkling and |
| | | | roughness; |
| | | | 5. Nicotinamide riboside-helps restore NAD+ levels which have been |
| | | | shown to reverse key indicators of aging by reducing wrinkles, improving |
| | | | moisture retention, and smoothening skin texture; |
| | | | 6. Nicotinamide mononucleotide-increases NAD+ levels which have |
| | | | been shown to reverse key indicators of aging by reducing wrinkles, |
| | | | improving moisture retention, and smoothening skin texture; |
| | | | 7. Coenzyme Q10-increases elastin production that tightens the skin, |
| | | | improves elasticity, and reduces fine lines and wrinkles; |
| | | | 8. Vitamin C-boosts collagen synthesis which helps reduce fine lines |
| | | | and wrinkles |
| | rs1801133 | Mutation leads to a folate | 1. Folate-improves folate deficiency, maintains skin barrier function by |
| | (MTHFR gene) | deficiency resulting in poor skin | retaining moisture and alleviates skin dryness, prevents premature skin |
| | | moisturization and elasticity | aging by enhancing collagen production; |
| | | which may accelerate skin aging. | 2. Curcumin-quenches free radicals, reduces oxidative stress, and |
| | | | prevents skin damage; |
| | | | 3. Epigallocatechin gallate-slows down the process of skin aging by |
| | | | reducing wrinkles, improving moisture retention, and smoothening skin |
| | | | texture; |
| | | | 4. Collagen-helps decelerate skin aging by reducing wrinkling and |
| | | | roughness; |
| | | | 5. Nicotinamide riboside-helps restore NAD+ levels which have been |
| | | | shown to reverse key indicators of aging by reducing wrinkles, improving |
| | | | moisture retention, and smoothening skin texture; |
| | | | 6. Nicotinamide mononucleotide-increases NAD+ levels which have |
| | | | been shown to reverse key indicators of aging by reducing wrinkles, |
| | | | improving moisture retention, and smoothening skin texture; |
| | | | 7. Coenzyme Q10-increases elastin production that tightens the skin, |
| | | | improves elasticity, and reduces fine lines and wrinkles; |
| | | | 8. Vitamin C-boosts collagen synthesis which helps reduce fine lines |
| | | | and wrinkles. |
| | rs 174547 | Mutation leads to an omega-3 | 1. DHA-is an anti-inflammatory factor and an antioxidant that helps |
| | (FADS1 gene) | fatty acid deficiency resulting in | reduce the signs of skin inflammation such as rash, redness, and irritation; |
| | | increased skin inflammation. | 2. EPA-is an anti-inflammatory factor and an antioxidant that helps |
| | | | reduce the signs of skin inflammation such as rash, redness, and irritation; |
| | | | 3. Vitamin C-helps reduce the signs of skin inflammation such as rash, |
| | | | redness, and irritation; |
| | | | 4. Curcumin-quenches free radicals, reduces oxidative stress and skin |
| | | | inflammation; |
| | | | 5. Green tea-contains polyphenols, that have anti-inflammatory and |
| | | | antioxidant properties which reduce skin inflammation and protect the skin |
| | | | from damage. |
| | | | 6. Vitamin E-is a strong antioxidant preventing skin inflammation |
| | | | caused due to lipid peroxide irritation. |

TABLE 7

| Category | Gene | Function | Methylation-Related Effect | Weight |
|---|---|---|---|---|
| Anti-Aging and Wrinkle | CDKN2A | Cell cycle regulation, cellular senescence | Increased methylation leads to cellular aging and reduced | 0.25 |
| Reduction | | | cell proliferation. | |
| | COL1A1 | Collagen production (extracellular matrix) | Methylation decreases collagen production, leading to | 0.3 |
| | | | wrinkles. | |
| | MMP1 | Collagen breakdown (matrix | High methylation levels increase collagen breakdown, | 0.25 |
| | | metalloproteinase) | contributing to aging signs. | |
| | SIRT1 | DNA repair, longevity, and metabolic | Promoter methylation reduces SIRT1 expression, affecting | 0.2 |
| | | regulation | skin longevity. | |
| Lymphatic Drainage | NFAT5 | Skin hydration and barrier function | Methylation affects expression, influencing hydration and | 0.25 |
| | | | lymphatic health. | |
| | VEGFC | Stimulate formation of Lymphatic vessels. | Dysfunction in VEGFC would lead to poor drainage of | 0.5 |
| | | | facial lymph, causing swelling and puffiness | |
| | PROX1 | Development and maintenance of | Hypermethylation leads to reduced expression of PROX1, | 0.25 |
| | | lymphatic endothelial cells. | impairing lymphatic vessel formation and maintenance. | |
| Skin Firming and | ELN | Elastin production (skin elasticity) | Methylation reduces elastin production, causing skin to | 0.25 |
| Elasticity | | | lose firmness and elasticity. | |
| | KLF4 | Stem cell maintenance, differentiation, and | Methylation affects skin stem cell function and firming | 0.15 |
| | | proliferation | capacity. | |
| | WRN | DNA repair, genomic stability | Methylation leads to decreased DNA repair capacity, | 0.25 |
| | | | which impacts skin elasticity. | |
| Brightening and | IGF2 | Growth factor, cell proliferation, survival | Methylation can influence pigmentation regulation | 0.15 |
| Pigmentation | | | through cellular growth pathways. | |
| | TYR | Tyrosinase, melanin synthesis | Methylation reduces expression of tyrosinase, decreasing | 0.2 |
| | | | melanin production. | |
| UV/Oxidative Damage/ | TP53 | Tumor suppressor gene, DNA damage | Methylation in the promoter region reduces TP53 func- | 0.3 |
| DNA/Cell Damage | | repair | tion, impairing DNA repair and increasing damage. | |
| | BCL2 | Regulates apoptosis and cellular survival | Methylation at BCL2's promoter can decrease apoptosis, | 0.25 |
| | | | causing cells to survive under stress. | |
| | SYN1 | Extracellular matrix and cell adhesion | Methylation can affect cell repair after UV-induced | 0.2 |
| | | | damage. | |
| Anti-inflammatory and | TNF-α | Pro-inflammatory cytokine | Methylation in the promoter region can reduce TNF-a | 0.25 |
| Soothing | | | expression, alleviating inflammation. | |
| | CXCL8 | Chemokine involved in inflammatory | Methylation at CpG sites can decrease CXCL8 | 0.2 |
| | | responses | expression, reducing inflammation. | |
| | IL-6 | Pro-inflammatory cytokine, involved in | Methylation modifications can reduce IL-6 expression, | 0.25 |
| | | aging and stress | thus minimizing inflammation. | |
| Hydration and Skin | FILAGGRIN | Skin barrier function, hydration | Methylation decreases filaggrin expression, impairing the | 0.3 |
| Barrier Repair | (FLG) | | skin barrier and hydration. | |
| | TGM1 | Transglutaminase, involved in skin barrier | Methylation reduces TGM1 expression, affecting skin's | 0.25 |
| | | formation | ability to form a proper barrier. | |
| | CLDN1 | Claudin-1, tight junction formation, skin | Methylation of CLDN1 reduces skin barrier function by | 0.25 |
| | | barrier | disrupting tight junctions. | |
| | AQP3 | Aquaporin-3, water transport, hydration | Methylation at AQP3′s promoter reduces water transport | 0.2 |
| | | | in the skin, leading to dryness. | |

TABLE 8
Skin Glycation

| Metabolite | Clinical Significance | Sample |
|---|---|---|
| Carboxymethyllysine (CML) | CML is a type of AGE that has | Urine |
| | been shown to increase linearly | |
| | with age in skin collagen. The | |
| | presence of CML and other | |
| | AGEs in skin collagen can | |
| | contribute to skin aging and | |
| | related disorders such as | |
| | wrinkles, age spots, and reduced | |
| | elasticity. | |
| Carboxyethyllysine (CEL) | CEL is a type of AGE that has | Urine |
| | been shown to increase linearly | |
| | with age in skin collagen. The | |
| | presence of CEL and other AGEs | |
| | in skin collagen can contribute to | |
| | skin aging and related disorders | |
| | such as wrinkles, age spots, and | |
| | reduced elasticity. | |
| Pentosidine | Pentosidine is an AGE that is | Urine |
| | particularly prevalent in skin | |
| | collagen and has been shown to | |
| | increase linearly with age. The | |
| | presence of pentosidine in skin | |
| | collagen can contribute to skin | |
| | aging and related disorders such | |
| | as wrinkles, age spots, and | |
| | reduced elasticity. | |
| Glucosepane | Glucosepane is a highly stable | Plasma |
| | and abundant AGE in human | |
| | tissues and is known to | |
| | accumulate with age. | |
| | Glucosepane is particularly | |
| | interesting as a biomarker of skin | |
| | glycation as it is found in high | |
| | concentrations in the extracellular | |
| | matrix of the skin, where it can | |
| | contribute to skin aging and | |
| | wrinkling. | |

TABLE 9

| Condition | Region | Directionality | Skin Microbiota (Bacteria) |
|---|---|---|---|
| Acne vulgaris | Forehead | ↑ | Propionibacterium acnes |
| | | | Propionibacterium avidum |
| | | | Propionibacterium granulosum |
| | Cheek | ↑ | Propionibacterium acnes |
| | Nasolabial Fold (Alar Crease) | ↑ | Staphylococcus epidermidis |
| | | | Corynebacterium spp. |
| | | | Propionibacterium acnes |
| | Chin | ↑ | Propionibacterium acnes |
| | Face(overall) | ↑ | Firmicutes |
| Rosacea | Cheek | ↑ | Corynebacterium kroppenstedtiin |
| | | | Staphylococcus epidermidis |
| | Nasolabial Fold (Alar Crease) | ↑ | Corynebacterium kroppenstedtiin |
| | eyelid margins | ↑ | Staphylococcus epidermidis |
| Atopic dermatitis | Cheek | ↑ | Staphylococcus epidermidis |
| | Chin | ↑ | Corynebacterium species |
| Seborrheic dermatitis | Cheek | ↑ | Malassezia spp. (fungi) |
| Pigmented skin | Cheek | ↑ | Eikenella corrodens |
| | | ↓ | Cutibacterium |
| | | | Micrococcus luteus |
| | | | Kocuria |
| Increased sebum | Cheek | ↑ | Acinetobacter |
| Wrinkles | Cheek | ↑ | Corynebacterium |
| Skin Ageing | Forehead | ↑ | Corynebacterium amycolatum |
| | | | Proteobacteria |
| | | | Firmicutes |
| | | | Bacteroidetes |
| | | ↓ | Propionibacterium |
| | Cheek | ↓ | Propionibacterium |
| | Face(overall) | ↑ | Corynebacterium |
| | | ↓ | Lactobacillus |
| | | | Cutibacterium |

TABLE 10

| | | | From | |
|---|---|---|---|---|
| | From Picture | | Questionnaire | |
| | Min | Max | Min | Max |
| Lymphatic drainage | 1 | 10 | 0 | 3 |
| Skin Firming and Elasticity | 1 | 10 | 0 | 3 |
| Anti-Aging and Wrinkle Reduction | 1 | 10 | 0 | 6 |
| Brightening and Pigmentation | 1 | 10 | 0 | 3 |
| UV/Oxidative Damage/DNA/Cell | 1 | 10 | 0 | 6 |
| damage | | | | |
| Anti-inflammatory and Soothing | 1 | 10 | 0 | 6 |
| Hydration and Skin Barrier Repair | 1 | 10 | 0 | 8 |

TABLE 11

| | | Phenotype |
|---|---|---|
| | Points range | Age |
| Lymphatic drainage | 1 to 3 | 30 |
| | 3 to 5 | 40 |
| | 5 to 7 | 50 |
| | 7 to 10 | 60 |
| | 11 to 13 | 70 |
| Skin Firming and Elasticity | 1 to 3 | 30 |
| | 3 to 5 | 40 |
| | 5 to 7 | 50 |
| | 7 to 10 | 60 |
| | 11 to 13 | 70 |
| Anti-Aging and Wrinkle Reduction | 1 to 3 | 30 |
| | 3 to 6 | 40 |
| | 7 to 10 | 50 |
| | 11 to 13 | 60 |
| | 14 to 16 | 70 |
| Brightening and Pigmentation | 1 to 3 | 30 |
| | 3 to 5 | 40 |
| | 5 to 7 | 50 |
| | 7 to 10 | 60 |
| | 11 to 13 | 70 |
| UV/Oxidative Damage/DNA/Cell | 1 to 3 | 30 |
| damage | 3 to 6 | 40 |
| | 7 to 10 | 50 |
| | 11 to 13 | 60 |
| | 14 to 16 | 70 |
| Anti-inflammatory and Soothing | 1 to 3 | 30 |
| | 3 to 6 | 40 |
| | 7 to 10 | 50 |
| | 11 to 13 | 60 |
| | 14 to 16 | 70 |
| Hydration and Skin Barrier Repair | 1 to 3 | 30 |
| | 3 to 6 | 40 |
| | 7 to 10 | 50 |
| | 11 to 14 | 60 |
| | 15 to 18 | 70 |

TABLE 12

| Phenotypic | | | |
|---|---|---|---|
| Parameters | Topicals | Supplements | Lifestyle |
| Wrinkles | 1. Tretinoin - induces the biosynthesis of | 1. Zeaxanthin - improves hydration | 1. Avoid tanning beds |
| | collagen and reduces the expression of MMP 1 | and reduces fine lines in facial skin as it | 2. Avoid smoking and |
| | (collagenase 1) thereby reducing fine lines on the | boosts the production of hyaluronic acid. | drinking alcohol |
| | skin. | 2. Collagen - reduces fine lines in | 3. Exercise regularly |
| | 2. Zeaxanthin - boosts hyaluronic acid, | the skin by promoting collagen synthesis, | 4. Cleanse your skin gently |
| | which can reduce the appearance of fine lines and | improving skin hydration and elasticity. | 5. Use a moisturizer every |
| | improve skin elasticity | 3. Vitamin C - reduces the | day |
| | 3. Retinol - inhibits collagenase and matrix | appearance of fine lines by enhancing | 6. Avoid sleeping on your |
| | metalloproteinases (MMP) expression; stimulates | collagen synthesis. | stomach or side to avoid skin |
| | collagen type 1 and glycosaminoglycans (GAGs) | 4. Resveratrol - has anti- | squishing or folding that may |
| | synthesis which helps reduce fine lines | inflammatory properties that may help to | accelerate wrinkle formation |
| | 4. Vitamin C - boosts collagen production | reduce the appearance of fine lines. | |
| | which reduces the appearance of fine lines | 5. Vitamin E - has antioxidant | |
| | 5. Hyaluronic acid - reduces the appearance | properties that can prevent wrinkle | |
| | of fine lines by increasing skin's hydration. | formation. | |
| Periorbital-around | 1. Caffeine - treats dark circles that result | 1. Vitamin C - boosts collagen | 1. Using a cold compress |
| eyes | from subcutaneous vascularisation, telangiectasias, | production, which in turn helps to | will help reduce puffy eyes |
| (puffy eyes) | and oedema due to leaky vessels. Caffeine-based gel | strengthen blood vessel walls and improve | 2. Reduce fluid intake |
| | penetrates the lower eyelid skin and diminishes the | their elasticity; reduces the risk of blood | before bedtime and limit salt in |
| | lower eyelid oedema and pigmentation. | pooling in the area, which can help to | your diet. |
| | 2. Phytonadione - improves blood flow and | minimize the appearance of dark circles. | 3. Get enough sleep and |
| | strengthens blood vessels, which can help to reduce | | sleep with your head slightly |
| | the appearance of dark circles. | | raised as it may help prevent |
| | 3. Retinol - increases cell turnover and | | fluid from accumulating around |
| | stimulates collagen production, which can help to | | your eyes as you sleep |
| | improve skin texture and tone. | | 4. Address your allergies (if |
| | 4. Vitamin E - reduces periorbital puffiness | | any) they can cause the eyes to |
| | by improving lymphatic drainage; improves | | redden, swell, and puff up |
| | lymphatic function by reducing inflammation and | | 5. Avoid smoking and |
| | increasing blood flow, which can help to reduce the | | drinking alcohol |
| | appearance of puffiness around the eyes; reduces the | | 6. Intake of potassium may |
| | appearance of dark circles by improving skin | | help reduce excess fluids in your |
| | hydration and elasticity. | | body |
| | 5. Astaxanthin - reduces puffiness under the | | 7. Do not sleep with |
| | eyes by reducing inflammation and oxidative stress | | makeup on. This may irritate |
| | which can lead to the accumulation of fluids and | | your eyes causing them to water, |
| | blood in the tissues around the eyes (cause puffiness | | leading to puffiness |
| | and dark circles). | | |
| Nasolabial folds | 1. Retinol - inhibits collagenase and matrix | 1. Vitamin E - has antioxidant | 1. Consume a healthy diet |
| (Laugh lines) | metalloproteinases (MMP) expression; stimulates | properties which can help reduce laugh | and exercise regularly |
| | collagen type 1 and glycosaminoglycans (GAGs) | lines of facial skin. | 2. Avoid tanning beds |
| | synthesis which helps reduce laugh lines. | 2. Collagen - reduces laugh lines in | 3. Avoid smoking and |
| | 2. Hyaluronic acid - reduces oxidative stress | the skin by promoting collagen synthesis, | drinking alcohol |
| | and may help to prevent damage to the skin and | improving skin hydration and elasticity, | 4. Sleep on your back as |
| | reduce the appearance of laugh lines. | and potentially reducing collagen | this may help gravity work |
| | 3. Peptides - promote collagen which may | degradation. | against laugh lines |
| | help improve the appearance of laugh lines. | 3. Vitamin C - helps reduce the | 5. Massaging your face in |
| | | appearance of laugh lines by boosting | an upward direction may help |
| | | collagen synthesis. | 6. Stay hydrated |
| | | 4. Resveratrol - has anti- | 7. Moisturize your skin |
| | | inflammatory properties that may help to | 8. Facial exercises may |
| | | reduce the appearance of laugh lines. | help |
| Worry Lines | 1. Retinoids - fight forehead lines and other | 1. Collagen - reduces worry lines in | 1. Wear sunglasses to avoid |
| | signs of aging; stimulates collagen production, | the skin by promoting collagen synthesis; | frowning in the sun. |
| | increases cell turnover, and provides antioxidant | improves skin hydration and elasticity. | 2. Avoid smoking and |
| | protection. | 2. Hyaluronic acid - has | drinking alcohol |
| | 2. Vitamin C - reduces the appearance of fine | antioxidant properties that can help to | 3. Sleep on your back as |
| | forehead lines by collagen synthesis, which is the | protect against free radicals; neutralizing | this may help gravity work |
| | process of creating new collagen molecules. | free radicals may help prevent further | against laugh lines |
| | 3. Hyaluronic acid - has antioxidant | damage to the skin and reduce the | 4. Stay hydrated |
| | properties that can help to protect against free | appearance of worry lines. | 5. Moisturize your skin |
| | radicals; neutralizing free radicals may help prevent | 3. Vitamin C - has antioxidant | 6. Facial exercises may |
| | further damage to the skin and reduce the appearance | activities and supports collagen | help |
| | of worry lines. | production, which can help reduce the | |
| | | appearance of worry lines. | |
| | | 4. Vitamin E - has antioxidant | |
| | | properties, that help protect the skin from | |
| | | damage caused by free radicals, which are | |
| | | unstable molecules that can damage cells | |
| | | and contribute to aging. | |
| Uneven Skin | 1. Niacinamide - reduces pigmentation by | 1. Vitamin C - inhibits melanin | 1. Always protect your skin |
| tone/Pigmentation | inhibiting the transfer of melanin from melanocytes | production by interfering with the activity | using a sunscreen |
| | to keratinocytes. It does this by reducing the | of an enzyme called tyrosinase, which is | 2. Limit the time your time |
| | expression of a protein called TRP-1, which is | essential for the production of melanin; | in the sun (especially between 10 |
| | involved in the transfer process; may also decrease | helps reduce skin pigmentation. | am-2 pm) |
| | melanosome production and distribution within | 2. Glutathione - reduces skin | 3. Avoid scratching or |
| | melanocytes. | pigmentation by suppressing free radicals, | picking your skin as it can result |
| | 2. Azelaic acid - reduces the appearance of | which enhance tyrosinase activity and | in inflammation and lead to |
| | dark spots and hyperpigmentation by inhibiting the | melanin synthesis. Higher levels of | darker pigmentation |
| | production of melanin. | pheomelanin are associated with lighter | |
| | 3. Vitamin C - inhibits melanin production by | skin colour, and higher levels of eumelanin | |
| | interfering with the activity of an enzyme called | cause darker skin colour. Glutathione | |
| | tyrosinase, which is essential for the production of | increases the pheomelanin/eumelanin ratio, | |
| | melanin. By blocking tyrosinase activity, vitamin C | resulting in lighter skin. | |
| | reduces the amount of melanin that is produced, | 3. Vitamin E - has antioxidant | |
| | which can help reduce skin pigmentation. | capabilities that reduce free radicals which | |
| | 4. Soybean extract - has isoflavones which | may lead to hyperpigmentation. | |
| | have been found to inhibit tyrosinase activity, which | | |
| | is the enzyme responsible for the production of | | |
| | melanin. This inhibition can lead to a reduction in | | |
| | the production of melanin, which in turn can lead to | | |
| | a reduction in pigmentation. | | |
| | 5. Retinoids - block the transport of melanin | | |
| | to epidermal cells, which reduces pigmentation; | | |
| | decreases the number of melanocytes, thereby | | |
| | inhibiting melanogenesis, thereby preventing | | |
| | pigmentation. | | |
| | 6. Kojic acid - exhibits antioxidant properties, | | |
| | and inhibits tyrosinase. This inhibition can lead to a | | |
| | reduction in the production of melanin, which in turn | | |
| | can lead to a reduction in pigmentation. | | |
| | 7. Vitamin E - has antioxidant capabilities | | |
| | that reduce free radicals which may lead to | | |
| | hyperpigmentation. | | |
| | 8. Aloesin - reduces pigmentation by | | |
| | inhibiting the activity of enzymes involved in | | |
| | melanin production. | | |
| | 9. Turmeric - contains curcumin, which | | |
| | inhibits the activity of tyrosinase which is involved | | |
| | in melanin synthesis. | | |
| Age spots (Liver | 1. Tretinoin - increases cell turnover and | 1. Vitamin C - promotes collagen | 1. Wear sunglasses |
| spots) | stimulates collagen production, which can help to | synthesis, which can improve skin texture | 2. Limit the time your time |
| | reduce the appearance of liver spots; reduces the | and reduce the appearance of age spots. | in the sun (especially between 10 |
| | amount of melanin in the skin, which can make liver | 2. Vitamin E - an antioxidant that | am-2 pm) |
| | spots less visible. | can help protect the skin from UV damage | 3. Wear protective clothing |
| | 2. Niacinamide - inhibits the transfer of | and reduce the appearance of age spots. | 4. Always apply sunscreen |
| | melanin from melanocytes to the surrounding skin | 3. Niacinamide - inhibits the | (minimum SPF 30+) before |
| | cells, which can help to reduce age spots and | transfer of melanin from melanocytes to | entering the sun |
| | blotchiness. | the surrounding skin cells, which can help | |
| | | to reduce age spots and blotchiness. | |
| | | 4. Coenzyme Q10 - is antioxidant | |
| | | that reduces oxidative stress, thereby | |
| | | preventing the development of age spots | |
| | | and reducing the appearance of existing | |
| | | ones. | |

TABLE 13

| Category | Question | Answer Options | Points Assigned |
|---|---|---|---|
| Anti-Aging and Wrinkle | How often do you notice fine lines or | 0 = Never, 1 = Occasionally, 2 = | 0 = 0 pts, 1 = 1 pt, 2 = 2 pts, |
| Reduction | wrinkles on your skin? | Frequently, 3 = Always | 3 = 3 pts |
| Anti-Aging and Wrinkle | How deep are these wrinkles? | 0 = No wrinkles, 1 = only some fine lines, 2 = | 0 = 0 pts, 1 = 1 pt, 2 = 2 pts, |
| Reduction | | Some fine, some deep, 3=many deep lines | 3 = 3 pts |
| Skin Firming and Elasticity | How firm and plump does your skin | 0 = Not firm, 1 = Slightly firm, 2 = | 0 = 3 pts, 1 = 2 pts, 2 = 1 pt, |
| | feel when you touch it? | Moderately firm, 3 = Very firm | 3 = 0 pts |
| Anti-inflammatory and | Is your skin acne-prone? | 0 = No, 1 = Occasionally, 2 = Often, 3 = | 0 = 0 pts, 1 = 1 pt, 2 = 2 pts, |
| Soothing | | Always | 3 = 3 pts |
| Anti-inflammatory and | Do you experience stinging or burning | 0 = Never, 1 = Occasionally, 2 = | 0 = 0 pts, 1 = 1 pt, 2 = 2 pts, |
| Soothing | sensation on your skin? | Frequently, 3 = Always | 3 = 3 pts |
| Brightening and | Do you notice uneven skin tone or dark | 0 = Never, 1 = Occasionally, 2 = | 0 = 0 pts, 1 = 1 pt, 2 = 2 pts, |
| Pigmentation | spots on your skin? | Frequently, 3 = Always | 3 = 3 pts |
| Hydration and Skin Barrier | Is your skin dry? | 0 = No, 1 = Occasionally, 2 = Often, 3 = | 0 = 0 pts, 1 = 1 pt, 2 = 2 pts, |
| Repair | | Always | 3 = 3 pts |
| Hydration and Skin Barrier | How does your skin feel after cleansing | 0 = Tight, 1 = Normal, 2 = Hydrated | 0 = 2 pts, 1 = 1 pt, 2 = 0 pts |
| Repair | (tight, normal, hydrated)? | | |
| Hydration and Skin Barrier | How often are you exposed to high | 0 = Never, 1 = Occasionally, 2 = | 0 = 0 pts, 1 = 1 pt, 2 = 2 pts, |
| Repair | humidity or wind? | Frequently, 3 = Always | 3 = 3 pts |
| UV/Oxidative Damage/ | How much sun/light exposure do you | 0 = None, 1 = 0-30 min, 2 = 30-60 min, 3 | 0 = 0 pts, 1 = 1 pt, 2 = 2 pts, |
| DNA/Cell Damage | get per day? | = 1 hour | 3 = 3 pts |
| UV/Oxidative Damage/ | How often do you use sunscreen or sun | 0 = Never, 1 = Occasionally, 2 = Often, 3 = | 0 = 3 pts, 1 = 2 pt, 2 = 1 pts, |
| DNA/Cell Damage | protection (SPF)? | Always | 3 = 0 pts |
| Lymphatic drainage | Do you often experience puffiness or | 0 = Never, 1 = Occasionally, 2 = | 0 = 0 pts, 1 = 1 pt, 2 = 2 pts, |
| | swelling, especially around your eyes | Frequently, 3 = Always | 3 = 3 pts |
| | or face? | | |

TABLE 14

| | Questions | | | |
|---|---|---|---|---|
| Sr | from the | Outcome | | |
| No | SQ | Topical | Supplement | Lifestyle |
| 1 | UV | 1. Zinc oxide - works by forming a protective | 1. Polypodium leucotomos - protects agains solar | 1. Always protect your |
| | | barrier on the skin's surface that reflects and scatters | UV radiation by reducing its damaging effects. | skin by applying sunscreen |
| | | UV radiation; acts as a physical shield, preventing | 2. Vitamin E - an antioxidant that helps protect the | Wear protective clothing |
| | | the UV rays from penetrating the skin, thus both | skin from UV damage. | and sunglasses. |
| | | UVA and UVB rays. | 3. Vitamin C - helps to neutralize free radicals | 2. Limit the time your |
| | | 2. Titanium dioxide - works by forming a physical | generated by UV radiation; enhances collagen | time in the sun (especially |
| | | barrier on the skin that reflects and scatters UV | production that strengthens the skin's barrier and | between 10 am-2 pm). |
| | | radiation; acts as a protective shield, preventing the | improves its resilience against UV damage. | |
| | | UV rays from reaching the deeper layers of the skin; | 4. Niacinamide - prevents the penetration of UV | |
| | | effectively blocks UVB rays. | radiation; promotes DNA repair mechanisms and | |
| | | | supports the natural repair processes of the skin, | |
| | | | aiding in the recovery from UV-induced damage. | |
| | Anti- | 1. Tretinoin - induces the biosynthesis of collagen | 1. Zeaxanthin - improves hydration and reduces fine | 1. Avoid tanning beds. |
| | wrinkling | and reduces the expression of MMP 1 (collagenase | lines in facial skin as it boosts the production of | 2. Avoid smoking and |
| | | 1) thereby reducing fine lines on the skin. | hyaluronic acid. | drinking alcohol. |
| | | 2. Zeaxanthin - boosts hyaluronic acid, which can | 2. Collagen - reduces fine lines in the skin by | 3. Exercise regularly. |
| | | reduce the appearance of fine lines and improve skin | promoting collagen synthesis, improving skin | 4. Cleanse your skin |
| | | elasticity. | hydration and elasticity. | gently. |
| | | 3. Retinol - inhibits collagenase and matrix | 3. Vitamin C - reduces the appearance of fine lines | 5. Use a moisturizer every |
| | | metalloproteinases (MMP) expression; stimulates | byenhancing collagen synthesis. | day. |
| | | collagen type 1 and glycosaminoglycans (GAGs) | 4. Resveratrol - has anti-inflammatory properties that | 6. Avoid sleeping on your |
| | | synthesis which helps reduce fine lines. | may help to reduce the appearance of fine lines. | stomach or side to avoid |
| | | 4. Vitamin C - boosts collagen production which | 5. Vitamin E - has antioxidant properties which can | skin squishing or folding |
| | | reduces the appearance of fine lines. | prevent wrinkle formation. | that may accelerate |
| | | 5. Hyaluronic acid - reduces the appearance of fine | | wrinkle formation. |
| | | lines by increasing skin's hydration. | | |
| | Anti- | 1. Niacinamide - reduces pigmentation by | 1. Vitamin C - inhibits melanin production by | 1. Always protect your |
| | pigmenta- | inhibiting the transfer of melanin from melanocytes | interfering with the activity of an enzyme called | skin using a sunscreen. |
| | tion | to keratinocytes. It does this by reducing the | tyrosinase, which is essential for the production of | 2. Limit the time your |
| | | expression of a protein called TRP-1, which is | melanin; helps reduce skin pigmentation. | time in the sun (especially |
| | | involved in the transfer process; may also decrease | 2. Glutathione - reduces skin pigmentation by | between 10 am-2 pm). |
| | | melanosome production and distribution within | suppressing free radicals, which enhance tyrosinase | 3. Avoid scratching or |
| | | melanocytes. | activity and melanin synthesis. Higher levels of | picking your skin as it can |
| | | 2. Azelaic acid - reduces the appearance of dark | pheomelanin are associated with lighter skin colour, | result in inflammation and |
| | | spots and hyperpigmentation by inhibiting the | and higher levels of eumelanin cause darker skin | lead to darker |
| | | production of melanin. | colour. Glutathione increases the | pigmentation. |
| | | 3. Vitamin C - inhibits melanin production by | pheomelanin/eumelanin ratio, resulting in lighter | |
| | | interfering with the activity of an enzyme called | skin. | |
| | | tyrosinase, which is essential for the production of | 3. Vitamin E - has antioxidant capabilities that | |
| | | melanin. By blocking tyrosinase activity, vitamin C | reduce free radicals which may lead to | |
| | | reduces the amount of melanin that is produced, | hyperpigmentation. | |
| | | which can help reduce skin pigmentation. | | |
| | | 4. Soybean extract - has isoflavones which have | | |
| | | been found to inhibit tyrosinase activity, which is the | | |
| | | enzyme responsible for the production of melanin. | | |
| | | This inhibition can lead to a reduction in the | | |
| | | production of melanin, which in turn can lead to a | | |
| | | reduction in pigmentation. | | |
| | | 5. Retinoids - block the transport of melanin to | | |
| | | epidermal cells, which reduces pigmentation; | | |
| | | decreases the number of melanocytes, thereby | | |
| | | inhibiting melanogenesis, thereby preventing | | |
| | | pigmentation. | | |
| | | 6. Kojic acid - exhibits antioxidant properties, and | | |
| | | inhibits tyrosinase. This inhibition can lead to a | | |
| | | reduction in the production of melanin, which in turn | | |
| | | can lead to a reduction in pigmentation. | | |
| | | 7. Vitamin E - has antioxidant capabilities that | | |
| | | reduce free radicals which may lead to | | |
| | | hyperpigmentation. | | |
| | | 8. Aloesin - reduces pigmentation by inhibiting the | | |
| | | activity of enzymes involved in melanin production. | | |
| | | 9. Turmeric - contains curcumin, which inhibits | | |
| | | the activity of tyrosinase which is involved in | | |
| | | melanin synthesis. | | |
| | Moisturi- | 1. Hyaluronic acid - acts as a barrier to prevent | 1. Hyaluronan - increases the overall availability of | 1. Avoid taking baths with |
| | zation | water loss through evaporation; reduces trans | hyaluronic acid in the body, leading to improved | hot water and use mild |
| | | epidermal water loss (TEWL); improves the skin's | skin hydration. Hyaluronan attracts and holds water | soaps. |
| | | ability to retain moisture, preventing dryness and | molecules, providing hydration to the skin from | 2. Limit your time in the |
| | | dehydration. | within and resulting in a more plump and | shower or bath to 5 or 10 |
| | | 2. Glycerin - creates a thin, occlusive layer that | moisturized appearance. | minutes. |
| | | helps to seal in moisture and prevent water loss | 2. Ceramides - provide additional ceramides | 3. Blot your skin gently |
| | | through evaporation; helps to maintain the skin's | (important for skin moisture) that can be incor- | dry with a towel, slather |
| | | hydration levels and prevent dryness. | poratedinto the skin's barrier, helping to strengthen | on the moisturizer |
| | | 3. Shea butter - resembles the natural lipids found | it and improve moisture retention. | immediately after drying |
| | | in the skin; replenishes the skin's lipid barrier, which | 3. Collagen - boosts collagen production which | your skin. |
| | | enhances moisture retention and improves the skin's | helps improve skin elasticity and moisture retention, | 4. Try using a humidifier. |
| | | ability to stay hydrated. | leading to hydrated and plump-looking skin. | |
| | | 4. Jojoba oil - resembles sebum naturally produced | | |
| | | by our skin; forms a non-greasy protective barrier on | | |
| | | the skin's surface, which helps to prevent trans | | |
| | | epidermal water loss (TEWL) and seals in moisture; | | |
| | | promotes hydration and softness. | | |
| | Radiant | 1. Vitamin C - promotes radiant skin by boosting | 1. Vitamin C - promotes radiant skin by boosting | 1. Gently wash your face |
| | complex- | collagen production, protecting against UV damage, | collagen production, protecting against UV damage, | when waking, before bed, |
| | ion | and acting as an antioxidant to neutralize free | and acting as an antioxidant to neutralize free | and after sweating. |
| | | radicals that can cause skin damage. | radicals that can cause skin damage. | 2. Have a balanced-diet |
| | | 2. Niacinamide - brightens skin by reducing the | | and drink plenty of water. |
| | | appearance of hyperpigmentation; inhibits the | | 3. Manage your stress by |
| | | transfer of melanin (the pigment responsible for skin | | exercising or meditating. |
| | | colour) to the skin cells, leading to a brighter, radiant | | |
| | | appearance. | | |
| 2 | Dry Skin | Glycerin - is a humectant that attracts and retains | | |
| | | moisture in the skin; strengthens the skin's natural | | |
| | | barrier, preventing moisture loss and protecting | | |
| | | against environmental stressors. | | |
| 2.1 | Mild dry | Glycerin × 1.0 | | |
| | Very dry | Glycerin × 1.5 | | |
| | Extremely | Glycerin × 2.0 | | |
| | dry | | | |
| 3 | Combina- | Hyaluronic acid - well-suited for combination skin | | |
| | tional | types as its hydrating properties help to restore | | |
| | skin - | moisture to dry areas, while its lightweight texture | | |
| | Dryness | makes it ideal for oily zones. | | |
| | and | | | |
| | oiliness | | | |
| 4 | Acne- | Benzoyl peroxide - has antibacterial properties that | | |
| | prone | help to kill acne-causing bacteria and reduce | | |
| | skin | inflammation; helps to unclog pores and exfoliate | | |
| | | the skin, making it an effective treatment for both | | |
| | | mild and severe acne. | | |
| 5 | Sun | 1. Zinc oxide - works by forming a protective | | |
| | exposure | barrier on the skin's surface that reflects and scatters | | |
| | | UV radiation; acts as a physical shield, preventing | | |
| | | the UV rays from penetrating the skin, thus both | | |
| | | UVA and UVB rays. | | |
| | | 2. Titanium dioxide - works by forming a physical | | |
| | | barrier on the skin that reflects and scatters UV | | |
| | | radiation; acts as a protective shield, preventing the | | |
| | | UV rays from reaching the deeper layers of the skin; | | |
| | | effectively blocks UVB rays. | | |
| | <1 hour | Zinc oxide + Titanium dioxide × 1.0 | | |
| | 1-2 hours | Zinc oxide + Titanium dioxide × 1.5 | | |
| | > 2 hours | Zinc oxide + Titanium dioxide × 2.0 | | |
| 6 | Skin= | | | |
| | treatments | | | |
| 7 | Sensitive | Niacinamide - well-suited for sensitive skin as it | | |
| | skin - | strengthens the skin barrier, reducing the likelihood | | |
| | stinging | of irritation and inflammation. | | |
| | or | | | |
| | burning | | | |
| 8 | Severe | | | |
| | dermato- | | | |
| | logical | | | |
| | concerns | | | |
| | Excessive | | | |
| | scratching | | | |
| | Excessive | | | |
| | Peeling | | | |
| | Pus and | | | |
| | oozing | | | |
| | (The | | | |
| | product is | | | |
| | not for | | | |
| | them) | | | |
| 9 | External | | | |
| | factors | | | |
| | No | | | |
| | Sun and | Zinc oxide - has broad-spectrum UVA and UVB | 1. Astaxanthin - an effective antioxidant that blocks | 1. Always use a sunscreen |
| | Humidity | protection, is soothing and gentle on sensitive skin, | UV radiation; protects against sunburn; acceleratese | and wear protective |
| | | non-comedogenic, which means it won't clog pores | the healing of skin after sun damage. | clothing. |
| | | or cause acne, an aspect that is increased during | 2. Vitamin E - plays an important role in | |
| | | sunny and sweaty weather. | photoprotection, preventing UV-induced free radical | |
| | | | damage to the skin. | |
| | | | 3. Omega-3 fatty acids - reduces the skin's | |
| | | | sensitivity to UV rays and inflammation caused | |
| | | | by sun exposure. | |
| | Wind and | Petroleum jelly - treats the effects of cold and wind | 1. Omega-3 fatty acids - reduces inflammation and | 1. Rehydrate and |
| | Cold | on the skin by forming a protective layer and | improves skin hydration, potentially preventing | moisturize skin regularly. |
| | | preventing the loss of moisture. | dryness, flakiness, and other skin damage caused by | |
| | | | cold temperatures and harsh winds. | |
| | | | 2. Probiotics - may improve skin barrier function, | |
| | | | increase hydration, and reduce inflammation, which | |
| | | | could help protect the skin from damage caused by | |
| | | | windy and cold temperatures. | |
| | Others | | | |
| 10 | Poor sleep | | 1. Melatonin - helps promote a regular sleep cycle. | 1. The quantity and |
| | | | 2. Valerian - reduces the amount of time it takes to | quality of sleep also affect |
| | | | fall asleep and helps one sleep better. | the skin. |
| | | | 3. Chamomile - acts as a mild sedative to calm | 2. Sleep for 8 hours. |
| | | | nerves, reduce anxiety, and improve insomnia. | 3. Set a routine, exercise |
| | | | | daily, and wake up at the |
| | | | | same time each day. |
| | | | | 4. Avoid caffeine late in |
| | | | | the day. |
| | | | | 5. Avoid alcohol and |
| | | | | usage of devices close to |
| | | | | bedtime. |

TABLE 15
HormonePro Test for male

| Hormone | Directionality | Supplements | Dosage |
|---|---|---|---|
| Estrone (E1) | ↓ | | |
| | ↑ | Calcium D-glucarate | 500 mg/day |
| | | Green Tea | 1,200 mL/day |
| | | Maca | 3 g/day |
| | | DIM (3,3′- | 200 mg/day |
| | | diindolylmethane) | |
| Estradiol (E2) | ↓ | Black Cohosh | 40 mg/day |
| | | Ginger | 0.5 g/day |
| | | Cranberry | 600 mg/day |
| | | Milk Thistle | 20 mg/day |
| | | Hops | 300 mg/day |
| | | Red clover | 40 mg/day |
| | | Damiana | 400 mg/day |
| | | Chaste Tree Berry | 200 mg/day |
| | | Extract | |
| | ↑ | Calcium D-glucarate | 500 mg/day |
| | | Green Tea | 1,200 mL/day |
| | | Maca | 3 g/day |
| | | DIM (3,3′- | 200 mg/day |
| | | diindolylmethane) | |
| | | Vitamin B2 | 1.3 mg/day |
| DHEA | ↓ | Fenugreek seed | 0.6 g/day |
| | | Ashwagandha | 1000 mg/day |
| | | Ginger | 0.5 g/day |
| | | Vitamin D3 | 600 IU/day |
| | | Zinc | 11 mg/day |
| | | Tribulus | 750 mg/day |
| | | Shatavari | 1000 mg/day |
| | ↑ | | |
| Testosterone | ↓ | Ginger | 0.5 g/day |
| | | Vitamin D3 | 600 IU/day |
| | | Zinc | 11 mg/day |
| | | Tribulus | 750 mg/day |
| | | Shatavari | 1000 mg/day |
| | ↑ | DHA | 1 g/day |
| | | EPA | 1 g/day |
| Epi-Testosterone | ↓ | Ginger | 0.5 g/day |
| (Epi-T) | | Vitamin D3 | 600 IU/day |
| | | Zinc | 11 mg/day |
| | | Tribulus | 750 mg/day |
| | ↑ | | |
| Androstenedione | ↓ | Ginger | 0.5 g/day |
| (AD) | | Vitamin D3 | 600 IU/day |
| | | Zinc | 11 mg/day |
| | | Tribulus | 750 mg/day |
| | | Shatavari | 1000 mg/day |
| | ↑ | Licorice Root Extract | 330 mg/day |
| Androsterone | ↓ | | |
| | ↑ | DHA | 1 g/day |
| | | EPA | 1 g/day |
| Etiocholanolone | ↓ | | |
| | ↑ | Licorice Root Extract | 330 mg/day |
| 5a-DHT | ↓ | Fenugreek seed | 0.6 g/day |
| | | Ashwagandha | 1000 mg/day |
| | | Vitamin D3 | 600 IU/day |
| | ↑ | Zinc | 11 mg/day |
| DHEA-S | ↓ | | |
| | ↑ | | |
| Progesterone (P) | ↓ | Vitamin D3 | 600 IU/day |
| | | Oregano | 5 ml/day |
| | | Curcumin | 1.5 g/day |
| | | Red clover | 40 mg/day |
| | ↑ | | |
| Cortisol | ↓ | Sodium | 1g/day |
| | ↑ | Ashwagandha | 1000 mg/day |
| | | Rhodiola rosea | 600 mg/day |
| Cortisone | ↓ | Vitamin B6 | 1.3 mg/day |
| | | Vitamin B12 | 2.4 mcg/day |
| | | Folate | 400 mcg/day |
| | ↑ | Calcium | 1,000 mg/day |
| | | Vitamin D3 | 600 IU/day |
| b-Tetrahydrocortisol | ↓ | Iodine | 150 mcg /day |
| (b-THF) | | Selenium | 55 mcg/day |
| | | Zinc | 11 mg/day |
| | ↑ | Ashwagandha | 1000 mg/day |
| | | Rhodiola rosea | 600 mg/day |
| a-Tetrahydrocortisol | ↓ | Iodine | 150 mcg /day |
| (a-THF) | | Selenium | 55 mcg/day |
| | | Zinc | 11 mg/day |
| | ↑ | Ashwagandha | 1000 mg/day |
| | | Rhodiola rosea | 600 mg/day |
| b-Tetrahydrocortisone | ↓ | Iodine | 150 mcg /day |
| (b-THE) | | Selenium | 55 mcg/day |
| | | Zinc | 11 mg/day |
| | ↑ | Ashwagandha | 1000 mg/day |
| | | Rhodiola rosea | 600 mg/day |
| Deoxycorticosterone | ↓ | | |
| | ↑ | Potassium | 3,400 mg/day |
| | | Calcium | 1,000 mg/day |
| | | Magnesium | 400 mg/day |
| Corticosterone | ↓ | | |
| | ↑ | Ashwagandha | 1000 mg/day |
| Melatonin | ↓ | Melatonin | 10 mg/day |
| | | Huang-qin | 3 g/day |
| | | St. John's-wort | 900 mg/day |
| | ↑ | | |
| 8-OHdG | ↓ | | |
| | ↑ | | |
| E3/(E1 + E2) Ratio | ↓ | | |
| | ↑ | | |
| 2-OH (E1 + E2)/16a- | ↓ | Iodine | 150 mcg /day |
| OH E1 | ↑ | | |
| 2-MeO E1/2-OH E1 | ↓ | Vitamin B6 | 1.3 mg/day |
| | | Vitamin B12 | 2.4 mcg/day |
| | | Folate | 400 mcg/day |
| | | Trimethylglycine | 500 mg/ day |
| | | (betaine) | |
| | ↑ | | |
| 4-MeO E1/4-OH E1 | ↓ | Vitamin B6 | 1.3 mg/day |
| | | Vitamin B12 | 2.4 mcg/day |
| | | Folate | 400 mcg/day |
| | | Trimethylglycine | 500 mg/ day |
| | | (betaine) | |
| | ↑ | | |
| 4-MeO E2/4-OH E2 | ↓ | Vitamin B6 | 1.3 mg/day |
| | | Vitamin B12 | 2.4 mcg/day |
| | | Folate | 400 mcg/day |
| | | Trimethylglycine | 500 mg/ day |
| | ↑ | (betaine) | |
| T/Epi-T | ↓ | | |
| | ↑ | | |
| b-Pregnanediol/E2 | ↓ | Calcium D-glucarate | 500 mg/day |
| | ↑ | | |
| Cortisol/cortisone | ↓ | | |
| | ↑ | | |
| Metabolized Cortisol | ↓ | Iodine | 150 mcg /day |
| (THF + THE) | | Selenium | 55 mcg/day |
| | | Zinc | 11 mg/day |
| | ↑ | Ashwagandha | 1000 mg/day |
| | | Rhodiola rosea | 600 mg/day |
| Pg/E2 | ↓ | Calcium D-glucarate | 500 mg/day |
| | ↑ | | |

TABLE 16
HormonePro Test for female

| Hormone | Directionality | Supplements | Dosage | |
|---|---|---|---|---|
| Estradiol (E2) | ↓ | Kudzu root | 300 | mg/day |
| | | Diosgenin | 450 | mg/day |
| | | Genistein | 250 | mg/day |
| | | Fennel | 200 | mg/day |
| | | Black Cohosh | 40 | mg/day |
| | | Green Tea | 1,200 | mL/day |
| | | Maca | 3 | g/day |
| | ↑ | Calcium D-glucurate | 500 | mg/day |
| Estrone (E1) | ↓ | Kudzu root | 300 | mg/day |
| | | Diosgenin | 450 | mg/day |
| | | Genistein | 250 | mg/day |
| | | Fennel | 200 | mg/day |
| | | Black Cohosh | 40 | mg/day |
| | | Green Tea | 1,200 | mL/day |
| | | Maca | 3 | g/day |
| | ↑ | Calcium D-glucurate | 500 | mg/day |
| Estriol (E3) | ↓ | Black Cohosh | 40 | mg/day |
| | | Ginger | 0.5 | g/day |
| | | Cranberry | 600 | mg/day |
| | | Milk Thistle | 20 | mg/day |
| | | Hops | 300 | mg/day |
| | | Red clover | 40 | mg/day |
| | | Damiana | 400 | mg/day |
| | | Chaste Tree Berry Extract | 200 | mg/day |
| | ↑ | Calcium D-glucurate | 500 | mg/day |
| | | Green Tea | 1,200 | mL/day |
| | | Maca | 3 | g/day |
| | | DIM (3,3′-diindolylmethane) | 200 | mg/day |
| Total Estrogen | ↓ | Kudzu root | 300 | mg/day |
| | | Diosgenin | 450 | mg/day |
| | | Genistein | 250 | mg/day |
| | | Fennel | 200 | mg/day |
| | | Black Cohosh | 40 | mg/day |
| | | Green Tea | 1,200 | mL/day |
| | | Maca | 3 | g/day |
| | ↑ | Calcium D-glucurate | 500 | mg/day |
| 2-OH Estradiol | ↓ | Iodine | 150 | mcg/day |
| | ↑ | | | |
| 2-OH Estrone | ↓ | Rosemary | 500 | mg/day |
| | | Curcumin | 1.5 | g/day |
| | | Garlic | 2400 | mg/day |
| | | Green Tea | 1,200 | mL/day |
| | | DHA | 1 | g/day |
| | | EPA | 1 | g/day |
| | | Vitamin B6 | 1.3 | mg/day |
| | | Vitamin B12 | 2.4 | mcg/day |
| | | Folate | 400 | mcg/day |
| | ↑ | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| 4-OH Estradiol | ↓ | | | |
| | ↑ | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| 4-OH Estrone | ↓ | | | |
| | ↑ | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| 16α-OH Estrone | ↓ | Black Cohosh | 40 | mg/day |
| | | Ginger | 0.5 | g/day |
| | | Cranberry | 600 | mg/day |
| | | Milk Thistle | 20 | mg/day |
| | | Hops | 300 | mg/day |
| | | Red clover | 40 | mg/day |
| | | Damiana | 400 | mg/day |
| | | Chaste Tree Berry Extract | 200 | mg/day |
| | ↑ | Flax seeds | 1 | g/day |
| | | DIM (3,3′- diindolylmethane) | 200 | mg/day |
| 2-MeO Estradiol | ↓ | Magnesium | 310 | mg/day |
| | | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| 2-MeO Estrone | ↓ | Magnesium | 310 | mg/day |
| | | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| 4-MeO Estradiol | ↓ | Magnesium | 310 | mg/day |
| | | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| 4-MeO Estrone | ↓ | Magnesium | 310 | mg/day |
| | | SAMe (S-adenosyl | 400 | mg/day |
| | | methionine) | | |
| | | Methionine | 19 | mg/kg/day |
| | | Vitamin B2 | 1.1 | mg/day |
| | | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| Bisphenol A | ↓ | | | |
| | ↑ | DHA | 1 | g/day |
| | | EPA | 1 | g/day |
| DHEA | ↓ | Fenugreek seed | 0.6 | gm/day |
| | | Ashwagandha | 1000 | mg/day |
| | | Ginger | 0.5 | g/day |
| | | Vitamin D3 | 600 | IU/day |
| | | Zinc | 8 | mg/day |
| | | Tribulus | 750 | mg/day |
| | | Shatavari | 1000 | mg/day |
| | | Zinc | 8 | mg/day |
| | | Tribulus | 750 | mg/day |
| | | Shatavari | 1000 | mg/day |
| | ↑ | Flax seeds | 1 | g/day |
| | | DHA | 1 | g/day |
| | | EPA | 1 | g/day |
| Androstenedione | ↓ | Fenugreek seed | 0.6 | gm/day |
| | | Ashwagandha | 1000 | mg/day |
| | | Ginger | 0.5 | g/day |
| | | Vitamin D3 | 600 | IU/day |
| | | Zinc | 8 | mg/day |
| | | Tribulus | 750 | mg/day |
| | | Shatavari | 1000 | mg/day |
| | | Zinc | 8 | mg/day |
| | | Tribulus | 750 | mg/day |
| | | Shatavari | 1000 | mg/day |
| | ↑ | Flax seeds | 1 | g/day |
| | | DHA | 1 | g/day |
| | | EPA | 1 | g/day |
| Androsterone | ↓ | pine nuts | 30 | gm/day |
| | ↑ | Flax seeds | 1 | g/day |
| | | DHA | 1 | g/day |
| | | EPA | 1 | g/day |
| Etiocholanolone | ↓ | Zinc | 8 | mg/day |
| | ↑ | | | |
| 5α-DHT | ↓ | | | |
| | ↑ | Zinc | 8 | mg/day |
| 5α,3α-Androstanediol | ↓ | | | |
| | ↑ | | | |
| 5β-Androstanediol | ↓ | | | |
| | ↑ | | | |
| DHEA-S | ↓ | | | |
| | ↑ | | | |
| Progesterone | ↓ | Vitamin C | 75 | mg/day |
| | | Oregano | 100 | gm/day |
| | | Curcumin | 1.5 | g/day |
| | | Red clover | 40 | mg/day |
| | | Damiana | 400 | mg/day |
| | ↑ | | | |
| Allopregnanolone | ↓ | Vitamin C | 75 | mg/day |
| | | Oregano | 100 | gm/day |
| | | Curcumin | 1.5 | g/day |
| | | Red clover | 40 | mg/day |
| | | Damiana | 400 | mg/day |
| | ↑ | | | |
| Allopregnanediol | ↓ | | | |
| | ↑ | | | |
| 3α-Dihydroprogesterone | ↓ | Zinc | 8 | mg/day |
| | ↑ | | | |
| 20α-Dihydroprogesterone | ↓ | | | |
| | ↑ | | | |
| b-Pregnanediol | ↓ | Vitamin C | 75 | mg/day |
| | | Oregano | 100 | gm/day |
| | | Curcumin | 1.5 | g/day |
| | | Red clover | 40 | mg/day |
| | | Damiana | 400 | mg/day |
| | ↑ | | | |
| a-Pregnanediol | ↓ | Vitamin C | 75 | mg/day |
| | | Oregano | 100 | gm/day |
| | | Curcumin | 1.5 | g/day |
| | | Red clover | 40 | mg/day |
| | | Damiana | 400 | mg/day |
| | ↑ | | | |
| Cortisol | ↓ | Sodium | 1 | g/day |
| | ↑ | Ashwagandha | 1000 | mg/day |
| | | Rhodiola rosea | 600 | mg/day |
| Cortisone | ↓ | Vitamin B6 | 1.3 | mg/day |
| | | Vitamin B12 | 2.4 | mcg/day |
| | | Folate | 400 | mcg/day |
| | ↑ | Calcium | 1,000 | mg/day |
| | | Vitamin D3 | 600 | IU/day |
| b-Tetrahydrocortisol | ↓ | Iodine | 150 | mcg/day |
| (b-THF) | | | | |
| | | Selenium | 55 | mcg/day |
| | | Zinc | 8 | mg/day |
| | ↑ | Ashwagandha | 1000 | mg/day |
| | | Rhodiola rosea | 600 | mg/day |
| a-Tetrahydrocortisol | ↓ | Iodine | 150 | mcg/day |
| (a-THF) | | | | |
| | | Selenium | 55 | mcg/day |
| | | Zinc | 8 | mg/day |
| | ↑ | Ashwagandha | 1000 | mg/day |
| | | Rhodiola rosea | 600 | mg/day |
| b-Tetrahydrocortisone | ↓ | Iodine | 150 | mcg/day |
| (b-THE) | | | | |
| | | Selenium | 55 | mcg/day |
| | | Zinc | 8 | mg/day |
| | ↑ | Ashwagandha | 1000 | mg/day |
| | | Rhodiola rosea | 600 | mg/day |
| Deoxycorticosterone | ↓ | | | |
| | ↑ | Potassium | 2,600 | mg/day |
| | | Calcium | 1,000 | mg/day |
| | | Magnesium | 310 | mg/day |
| Corticosterone | ↓ | | | |
| | ↑ | Ashwagandha | 1000 | mg/day |
| Melatonin | ↓ | Melatonin | 10 | mg/day |
| | | Huang-qin | 3 | g/day |
| | | St. John's-wort | 900 | mg/day |
| | ↑ | | | |
| 8-OHdG | ↓ | | | |
| | ↑ | | | |
| E3/(E1 + E2) Ratio | ↓ | | | |
| | ↑ | | | |
| 2-OH (E1 + E2)/16a-OH | ↓ | Iodine | 150 | mcg/day |
| E1 | | | | |
| | ↑ | | | |
| 2-MeO E1/2-OH E1 | ↓ | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| 4-MeO E1/4-OH E1 | ↓ | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| 4-MeO E2/4-OH E2 | ↓ | Vitamin B6 | 1.3 | mg/day |
| | | Vitamin B12 | 2.4 | mcg/day |
| | | Folate | 400 | mcg/day |
| | | Trimethylglycine (betaine) | 500 | mg/day |
| | ↑ | | | |
| T/Epi-T | ↓ | | | |
| | ↑ | | | |
| b-Pregnanediol/E2 | ↓ | Calcium D-glucurate | 500 | mg/day |
| | ↑ | | | |
| Cortisol/cortisone | ↓ | | | |
| | ↑ | | | |
| Metabolized Cortisol | ↓ | Iodine | 150 | mcg/day |
| (THF + THE) | | | | |
| | | Selenium | 55 | mcg/day |
| | | Zinc | 8 | mg/day |
| | ↑ | Ashwagandha | 1000 | mg/day |
| | | Rhodiola rosea | 600 | mg/day |
| Pg/E2 | ↓ | Calcium D-glucurate | 500 | mg/day |

↑

TABLE 17
MetabolicPro test

| | | | Dosage | | | |
|---|---|---|---|---|---|---|
| Category | Marker | Supplement | Male | | Female | |
| Serum markers | | | | | | |
| Lipid metabolism | Vaccenic acid | Conjugated | 3 | g/day | 3 | g/day |
| | | Linoleic Acid | | | | |
| | | DHA | 1 | g/day | 1 | g/day |
| | | EPA | 1 | g/day | 1 | g/day |
| | Adiponectin | DHA | 1 | g/day | 1 | g/day |
| | | EPA | 1 | g/day | 1 | g/day |
| | | Vitamin D | 600 | IU/day | 600 | IU/day |
| | | Curcumin | 1.5 | g/day | 1.5 | g/day |
| | | Berberine Extract | 400 | mg/day | 400 | mg/day |
| | | Resveratrol | 75 | mg/day | 75 | mg/day |
| | Acylhexosylceramide | Vitamin B3 | 16 | mg/day | 14 | mg/day |
| | (AHC) | | | | | |
| | | Vitamin B6 | 1.3 | mg/day | 1.3 | mg/day |
| | | Vitamin E | 22 | IU/day | 22 | IU/day |
| | Diacylglycerol (DAG) | Vitamin B3 | 16 | mg/day | 14 | mg/day |
| | | DHA | 1 | g/day | 1 | g/day |
| | | EPA | 1 | g/day | 1 | g/day |
| | Dimethyl- | Choline | 550 | mg/day | 425 | mg/day |
| | phosphatidylethanolamine | | | | | |
| | (DMPE) | | | | | |
| | PAI-1 | DHA | 1 | g/day | 1 | g/day |
| | | EPA | 1 | g/day | 1 | g/day |
| | | Vitamin D | 600 | IU/day | 600 | IU/day |
| Carbohydrate | Adiponectin | DHA | 1 | g/day | 1 | g/day |
| metabolism | | | | | | |
| | | EPA | 1 | g/day | 1 | g/day |
| | | Vitamin D | 600 | IU/day | 600 | IU/day |
| | | Curcumin | 1.5 | g/day | 1.5 | g/day |
| | | Berberine Extract | 400 | mg/day | 400 | mg/day |
| | | Resveratrol | 75 | mg/day | 75 | mg/day |
| | GIP | Vitamin D | 600 | IU/day | 600 | IU/day |
| | | Vitamin B3 | 16 | mg/day | 14 | mg/day |
| | | Vitamin B6 | 1.3 | mg/day | 1.3 | mg/day |
| | | Vitamin C | 90 | mg/day | 75 | mg/day |
| | Leptin | DHA | 1 | g/day | 1 | g/day |
| | | EPA | 1 | g/day | 1 | g/day |
| | | Vitamin D | 600 | IU/day | 600 | IU/day |
| Cellular | NAD | Vitamin B3 | 16 | mg/day | 14 | mg/day |
| metabolism | | | | | | |
| | | Tryptophan | 5 | mg/kg/day | 5 | mg/kg/day |
| | | Nicotinamide | 100 | mg/day | 100 | mg/day |
| | | riboside | | | | |
| | | Resveratrol | 75 | mg/day | 75 | mg/day |
| | | Quercetin | 250 | mg/day | 250 | mg/day |
| Urine markers | | | | | | |
| Lipid metabolism | Adipate | Vitamin B2 | 1.3 | mg/day | 1.1 | mg/day |
| | | L-carnitine | 300 | mg/kg/day | 300 | mg/kg/day |
| | Suberate | Vitamin B2 | 1.3 | mg/day | 1.1 | mg/day |
| | | L-carnitine | 300 | mg/kg/day | 300 | mg/kg/day |
| | Ethyl malonate | Vitamin B2 | 1.3 | mg/day | 1.1 | mg/day |
| | | Vitamin B1 | 1.2 | mg/day | 1.1 | mg/day |
| | | Vitamin B12 | 2.4 | mcg/day | 2.4 | mcg/day |
| | | CoQ10 | 100 | mg/day | 100 | mg/day |
| | | L-carnitine | 300 | mg/kg/day | 300 | mg/kg/day |
| Carbohydrate | Pyruvate | Vitamin B1 | 1.2 | mg/day | 1.1 | mg/day |
| metabolism | | | | | | |
| | | L-carnitine | 300 | mg/kg/day | 300 | mg/kg/day |
| | | CoQ10 | 100 | mg/day | 100 | mg/day |
| | | Alpha lipoic acid | 50 | mg/day | 50 | mg/day |
| | L-lactate | Vitamin B1 | 1.2 | mg/day | 1.1 | mg/day |
| | | Vitamin B2 | 1.3 | mg/day | 1.1 | mg/day |
| | β-hydroxybutyrate | Chromium | 35 | mcg/day | 25 | mcg/day |

TABLE 18
NutriLite
Serum Analytes

Folate (L-5-methyltetrahydrofolate)
Magnesium, Mg 24
Iron, Fe 56
Vitamin A (Retinol)
Vitamin D3 (Cholecalciferol)
Vitamin E
Choline
Free Carnitine
Vitamin B1 (Thiamine diphosphate)
Vitamin B2 (Riboflavin 5-Phosphate)
Vitamin B5 (Pantothenic acid)
Vitamin B6, Pyridoxal 5-Phosphate
Vitamin C (L-Ascorbic acid)
Copper, Cu 63
Zinc, Zn 67
Selenium, Se 76
Chromium, Cr 53
Manganese, Mn 55

TABLE 19
NutriPro Test

| | | Analysis | | | |
|---|---|---|---|---|---|
| | Analyte | Serum | RBC | WBC | Genetics |
| Vitamins | Vitamin A (Retinol) | Yes | | Yes | rs12934922, |
| and | | | | | rs1667255, |
| minerals | | | | | rs6564851, |
| | | | | | rs7501331, |
| | | | | | rs11645428 |
| | Vitamin A (beta-carotene) | Yes | | | rs11645428 |
| | Vitamin B1 (Thiamine diphosphate) | Yes | Yes | Yes | rs17514104 |
| | Vitamin B2 (Riboflavin 5-Phosphate) | Yes | Yes | Yes | rs1799983 |
| | Vitamin B3 (Nicotinic acid) | Yes | Yes | Yes | |
| | Vitamin B5 (Pantothenic acid) | Yes | | Yes | |
| | Vitamin B6, Pyridoxal 5-Phosphate | Yes | Yes | Yes | |
| | Vitamin B7 (Biotin) | Yes | | | rs13078881 |
| | Vitamin B12 (Cyanocobalamin) | Yes | Yes | Yes | rs492602, |
| | | | | | rs526934, |
| | | | | | rs602662 |
| | Vitamin C (L-Ascorbic acid) | Yes | Yes | Yes | rs33972313, |
| | | | | | rs4257763, |
| | | | | | rs6139591, |
| | | | | | rs6596473 |
| | Vitamin D, 25-OH | Yes | | | rs10741657, |
| | | | | | rs12785878, |
| | | | | | rs2282679, |
| | | | | | rs10766197 |
| | Vitamin D3 (Cholecalciferol) | Yes | | Yes | rs10877012 |
| | Vitamin D, 1-25 dihydroxy | Yes | | | rs4588 |
| | Vitamin E (alpha-tocopherol) | Yes | Yes | Yes | rs12272004 |
| | Vitamin K1 (Phylloquinone) | Yes | | Yes | rs2108622 |
| | Vitamin K2 (Menaquinone-MK-7) | Yes | | Yes | |
| | Folate (L-5-methyltetrahydrofolate) | Yes | Yes | | rs1801131, |
| | | | | | rs1801133 |
| | Coenzyme Q10 (Ubiquinone + Ubiquinol), Total | Yes | Yes | Yes | rs775607037, |
| | | | | | rs786204770 |
| | Selenium, Se 76 | Yes | Yes | Yes | rs1050450, |
| | | | | | rs3877899 |
| | Sodium | Yes | Yes | | rs2304478, |
| | | | | | rs7204044 |
| | Potassium | Yes | Yes | | rs889299 |
| | Calcium, Ca 44 | Yes | Yes | Yes | rs4516035 |
| | Zinc, Zn 67 | Yes | Yes | Yes | rs11126936 |
| | Manganese, Mn 55 | Yes | Yes | Yes | rs13107325 |
| | Iron, Fe 56 | Yes | Yes | | rs1799945, |
| | | | | | rs1800562, |
| | | | | | rs3811647, |
| | | | | | rs4820268, |
| | | | | | rs855791 |
| | Magnesium, Mg 24 | Yes | Yes | | rs4680 |
| | Copper, Cu 63 | Yes | Yes | Yes | rs76151636 |
| | Chromium, Cr 53 | Yes | Yes | | |
| | Myo-Inositol | Yes | | | |
| | Iodine | Yes | Yes | | rs225014 |
| | Molybdenum | Yes | Yes | | rs594445 |
| | Phosphorus | Yes | Yes | | rs4074995 |
| | Tetrahydrobiopterin | Yes | Yes | | rs5030853, |
| | | | | | rs8007267 |
| | Fluoride | Yes | | | rs4284505 |
| | Copper/Zinc | Yes | | | |
| Amino | Glutathione Oxidized | Yes | Yes | Yes | rs121909307, |
| Acids | | | | | rs1695 |
| | MMA (Methymalonic acid) | Yes | | | rs291466, |
| | | | | | rs121918252 |
| | Choline | Yes | | Yes | rs3733890, |
| | | | | | rs7946 |
| | L-Cysteine | Yes | | Yes | |
| | L-Asparagine | Yes | | Yes | |
| | L-Glutamine | Yes | | Yes | |
| | L-Serine | Yes | | Yes | |
| | L-Arginine | Yes | | | |
| | L-Citrulline | Yes | | | |
| | L-Isoleucine | Yes | | | |
| | L-Valine | Yes | | | |
| | L-Leucine | Yes | | | |
| | Free Carnitine | Yes | | Yes | |
| | Phenylalanine | | | | rs5030853 |
| Fatty | DHA (Docosahexaenoic acid) | Yes | Yes | | |
| Acids | EPA (Eicosapentaenoic acid) | Yes | Yes | | |
| | DPA (Docosapentaenoic acid) | Yes | Yes | | |
| | AA (Arachidonic acid) | Yes | Yes | | |
| | LA (Linoleic acid) | Yes | Yes | | rs174547 |
| | Omega-3 Total | | Yes | | |
| | Omega-6 Total | | Yes | | |
| | Omega-3 Index | | Yes | | |
| | AA/EPA | | Yes | | |

TABLE 20
Polymer/Solubilizing/Gelling/Emulsifier
Agents
Cellulose
Algin
Xanthan Gum
PEG Macademia Glycerides
Polyacrylate Crosspolymer 6
Ammonium Acryloyldimethyltaurate/VP
Copolymer
Caprylyl Glycol
Lecithin
Carbomer
Hydroxyethyl Acrylate/Sodium
Acryloyldimethyl Taurate Copolymer
Squalane
Humectant and Emolient
Glycerin
Sorbitol 70
Sodium Hylauronate
Preservative
Phenoxyethanol
Ethylhexylglycerin
Chelating agent
Sodium Phytate
Disodium EDTA
Penetration enhancers
Butylene Glycol
Pentylene Glycol
Sodium Lauryl sulfate
Dimethylisosorbide
Parfum
May be added

TABLE 21-1
Anti-Aging and Wrinkle Reduction

| Peptide Name | Sequence | Function/Benefit |
|---|---|---|
| Palmitoyl Pentapeptide-3 (Matrixyl) | Pal-KTTKS | Stimulates collagen synthesis, reduces fine lines. |
| Acetyl Dipeptide-1 Cetyl Ester (Idealift) | Ac-Tyr-Arg-O-hexadecyl ester | Firming, improves skin structure. |
| Acetyl Hexapeptide-8 (Argireline) | Ac-Glu-Glu-Met- Gln-Arg-Arg-NH2 | Reduces wrinkle depth by inhibiting muscle |
| | | contractions (“Botox-like”). |
| Acetyl Hexapeptide-30 (Inyline ®-Peptide) | Val-Gly-Val-Ala-Pro-Gly | Modulates botox like action |
| Acetyl Hexapeptide-51 Amide (Juveleven) | | Improves skin texture, reduces wrinkles. |
| Argirelox (Pentapeptide 18 + Argireline) | Tyr-D-Ala-Gly-Phe-Leu | Smooths wrinkles by relaxing facial muscles. |
| SNAP 8-Acetyl Octapeptide-3 | Ac-Glu-Glu-Met-Gln-Arg-Arg-Ala-Asp-NH2 | Muscle-relaxing peptide that mimics the effect |
| | | of Botulinum toxin (Botox) |
| Tripeptide 5 | Pal-KVK | Stimulate collagen production |
| GHK Cu | GHK-Cu | |
| reproage-Disodium Phosphate, Acetyl | | Argireline ™ NP and Reproage ™ have the same |
| Hexapeptide-8, Sodium Phosphate, | | INCI name, but they are two different molecules |
| Caprylyl Glycol | | with different sequences and completely |
| | | different mechanisms of action. This means, if |
| | | you choose, you can combine Argireline ™ NP |
| | | or SNAP 8 and Reproage ™ in the same |
| | | formulation. |
| Synake | beta-Ala-Pro-Dab-NHBzl | Syn-ake is inspired by WAGLERIN-1 |
| | | PEPTIDE. This is H G G K P D L R P C H C P P |
| | | Y H P R P K P I R O H |
| Palmitoyl Pentapeptide 38 | Pal-Lys-Met(O2)-Lys-OH | Matrikine mimic compound |

TABLE 21-2
Skin Firming and Elasticity

| Peptide Name | Sequence | Function/Benefit |
|---|---|---|
| Palmitoyl Hexapeptide-12 (Biopeptide EL) | Pal-VGVAPG | Skin firming and elasticity |
| Palmitoyl Oligopeptide (Bio-bustyl) | Pal-GHK | Improves skin elasticity and firmness. |
| Myristoyl Pentapeptide-8 (SymPeptide 225) | Ac-Lys-Gly-His-Lys-NH2 | Improves skin elasticity and firmness. |
| Acetyl Heptapeptide 4 (Fensebiome) | Ac-Glu-Glu-Met-Gln-Arg-Arg-Ala | Promotes skin elasticity and firmness. |
| Triptide-10 Citrulline (Decorinyl) | Lys-alpha-Asp-Ile-Citrulline | Targets skin hydration and structure. |
| Hexapeptide-10 (Serilesine) | Ser-Asp-Gly-Pro-Arg-Pro | Promotes collagen production, |
| | | enhances skin elasticity. |
| Acetyl Tetrapeptide-11 (Syniorage) | Ac-Pro-Pro-Tyr-Leu | Boosts skin elasticity and resilience. |
| Acetyl Hexapeptide-38 (Adifyline) | Ac-Ser-Val-Val-Val-Arg-Thr-NH2 | Volumizes and plumps skin. |
| Hexapeptide-11 (Peptide Vinci 2) | Phe-Val-Ala-Pro-Phe-Pro | Skin firming and elasticity |
| Acetyl Dipeptide-13 DIPHENYLGLYCINE | Ac-Arg-Trp-Phg-Phg-OH | Help sagging and elasticity |
| (Relistase) | | |
| Palmitoyl Dipeptide-5 Diaminobutyloyl | Pal-Lys-Val-Dab-OH | Skin firming and elasticity |
| Hydroxythreonine (and) Palmitoyl Dipeptide-6 | | |
| Diaminohydroxybutyrate (Syntacks) | | |
| Acetyl Tetrapeptide-2 (Uplevity) | Ac-Lys-Asp-Val-Tyr | Firms skin, enhances lifting effect. |

TABLE 21-3
Hydration and Skin Barrier Repair

| Peptide Name | Sequence | Function/Benefit |
|---|---|---|
| Decapeptide-4 | | Moisturizer |
| Acetyl Hexapeptide-37 (Diffuporine) | Ac-L-Ser-L-Pro-L-Ala-Gly-Gly-L-Pro-NH 2 | Improves skin permeability, |
| | | enhances hydration. |
| Oligopeptide-24 (EGF-like peptide) | Cys-Met-Tyr-Ile-Glu-Gly-Gly-Gly-Gly-Arg- | Mimics EGF, promotes skin |
| | Gly-Asp-Gly | renewal and repair. |
| FSS Ceramide 3 | | Moisturizer |
| SODIUM pca | | Humectant |
| APT-GPF | Ahnfeltiopsis Concinna Extract | Moisturizer |

TABLE 21-4
Anti-inflammatory and Soothing

| Peptide Name | Sequence | Function/Benefit |
|---|---|---|
| Acetyl Hexapeptide-49 (Delisens) | Ac-Phe-Phe-Trp-Phe-His-Val-OH | Calms and soothes sensitive skin. |
| Palmitoyl tetrapeptide-7 | Pal-Gly-Gln-Pro-Arg-OH | Anti-inflammatory |
| Acetyl Tetrapeptide-15 (Skinasensyl) | Tyr-Pro-Phe-Phe | Enhances skin barrier |
| | | and reduces irritation. |
| Polyquaternium-51 | | |
| Allantoin | 5-ureidohydantoin or glyoxyldiureide | |
| Hesperdine Methyl Chalcone | | Targets cytokines and anti-inflammatory |
| Acetyl Tetrapeptide-40 | Acetyl-Ala-Thr-Asn-Thr | Reduces skin redness and strengthens |
| (TELANGYN) | | blood vessels. |
| Acetyl Dipeptide-3 Aminohexanoate | Arg-Ala.Acetate | Body firming, skin smoothing. |
| (Bodyfensine) | | |
| Oligopeptide-10 | Phe-Ala-Lys-Ala-Leu-Lys-Ala-Leu- | Skin healing, acne reduction, |
| | Leu-Lys-Ala-Leu-Lys-Ala-Leu | antimicrobial properties. |
| Acetyl Tetrapeptide-2 (Syn-UP) | Ac-Lys-Asp-Val-Tyr | Redness |

TABLE 21-5
Brightening and Pigmentation

| Peptide Name | Sequence | Function/Benefit |
|---|---|---|
| Hexapeptide-2 | His-Trp-Ala-Trp-Phe-Lys | Brightens skin tone, reduces hyperpigmentation. |
| Nonapeptide-1 | H-Met-Pro-D-Phe-Arg-D-Trp-Phe-Lys-Pro-Val- | Reduces melanin production, brightens skin |
| (Melanostatine) | NH2 | tone. |
| Decapeptide 12 | Tyr-Arg-Ser-Arg-Lys-Tyr-Ser-Ser-Trp-Tyr | address skin tone unevenness, pigmentation |
| | | issues |
| Oligopeptide-34 | Val-Gly-Val-Ala-Pro-Gly | Brightens skin tone |
| Oligopeptide-51 | H-Tyr-Arg-Ser-Arg-Lys-Tyr-Ser-Ser-Trp-Tyr-OH | Brightens skin tone |

TABLE 21-6
Lymphatic drainage

| Peptide Name | Sequence | Function/Benefit |
|---|---|---|
| Eyeseryl-Acetyl Tetrapeptide-5 | Ac-β-{Ala}-His-Ser-His-OH | |
| Caffeine | | Anti-oxidant, Lymphatic drainage |

TABLE 21-7
UV/Oxidative Damage/DNA/Cell damage

| Peptide Name | Sequence | Function/Benefit |
|---|---|---|
| DAP Tripeptide 33 | {Dap}-Ala-Pro-His | |
| Pepha tight | | |
| Ferulic acid | | |
| Acetyl Tetrapeptide-22 | Ac-His-Leu-Leu-Arg | Protects skin from environmental stress and |
| (Thermostressine) | | damage. |
| Acetyl Heptapeptide-1 | Ac-Asp-Glu-Glu-Thr-Gly-Glu-Phe | Induce protection of Cell protecting enzymes |
| Tripeptide-9 Citrulline (dGlyage) | | Prevents glycation |

TABLE 21-8
Pre and Probiotics

| | |
|---|---|
| Pepha active | Probiotic |
| Beta Glucan | Prebiotic |
| Bacillus Extract | Probiotic |
| Sea kelp extract | Prebiotic |
| Alpaflor Sebum | Prebiotic |

TABLE 21-9
Vitamins

| | |
|---|---|
| D Panthenol | Vitamin B5 |
| Magnesium Ascorbyl Phosphate | Vitamin C |
| Tocopherols | Vitamin E |
| Niacinamide | Vitamin B3 |

TABLE 22-1
Anti-Aging and Wrinkle Reduction

| | | | | Average | |
|---|---|---|---|---|---|
| | | Methylation | Phenotype | (Methylation, | |
| | Genotype | Age | Age | Phenotype age) + | |
| Peptide Name | At least 1 genetic issue | 51-60 | 41-50 | Genotype | Scaled |
| Palmitoyl Pentapeptide-3 (Matrixyl) | 0.50% | 2% | 2% | 2.50% | 1.9% |
| Acetyl Dipeptide-1 Cetyl Ester (Idealift) | 1% | 3% | 2% | 3.50% | 2.6% |
| Acetyl Hexapeptide-8 (Argireline) | | | | | |
| Acetyl Hexapeptide-30 (Inyline ®-Peptide) | 1% | 3% | 2% | 3.50% | 2.6% |
| Acetyl Hexapeptide-51 Amide (Juveleven) | | 1% | 1% | 1.00% | 0.8% |
| Argirelox (Pentapeptide 18 + Argireline) | 1% | 7% | 5% | 7.00% | 5.3% |
| SNAP 8-Acetyl Octapeptide-3 | 0.50% | 3% | 2% | 3.00% | 2.3% |
| Tripeptide 5 | | 1% | 1% | 1.00% | 0.8% |
| GHK Cu | 0.50% | 0.50% | 0.50% | 1.00% | 0.8% |
| reproage-Disodium Phosphate, Acetyl | | 2% | 1% | 1.50% | 1.1% |
| Hexapeptide-8, Sodium Phosphate, | | | | | |
| Capryly lGlycol | | | | | |
| Synake | | 4% | 2% | 3.00% | 2.3% |
| Palmitoyl Pentapeptide 38 | 1% | 2% | 1% | 2.00% | 1.5% |

TABLE 22-2
Skin Firming and Elasticity

| | | | | Average | |
|---|---|---|---|---|---|
| | | | | (Methylation, | |
| | | | | Phenotype age) + | |
| | | Methylation | Phenotype | Genotype | |
| | Genotype | Age | Age | Average | |
| | At least 1 | 41-50 | 41-50 | (Methylation, | |
| | genetic issue | Methylation | Phenotype | Phenotype age) + | Scaled |
| Peptide Name | Genotype | Age | Age | Genotype | Scaled |
| Palmitoyl Hexapeptide-12 (Biopeptide EL) | | 3% | 3% | 3.00% | 2.3% |
| Palmitoyl Oligopeptide (Bio-bustyl) | | | | | |
| Improves skin elasticity and firmness. | | | | | |
| Palmitoyl Oligopeptide (Biopeptide CL) | | | | | |
| Acetyl Heptapeptide 4 (Fensebiome) | 0.25% | 1.00% | 1% | 1.25% | 0.9% |
| Triptide-10 Citrulline (Decorinyl) | 1.00% | 4% | 4% | 5.00% | 3.8% |
| Hexapeptide-10 (Serilesine) | 1% | 2% | | 3.00% | 2.3% |
| Acetyl Tetrapeptide-11 (Syniorage) | 1% | | | | |
| Acetyl Hexapeptide-38 (Adifyline) | | 1% | 1% | 1.00% | 0.8% |
| Hexapeptide-11 (Peptide Vinci 2) | | | | | |
| Acetyl Dipeptide-13 DIPHENYLGLYCINE | | | | | |
| (Relistase) | | | | | |
| Palmitoyl Dipeptide-5 Diaminobutyloyl | 0.50% | 0.50% | 0.50% | 1.00% | 0.8% |
| Hydroxythreonine (and) Palmitoyl | | | | | |
| Dipeptide-6 Diaminohydroxybutyrate | | | | | |
| (Syntacks) | | | | | |
| Acetyl Tetrapeptide-2 (Uplevity) | | 2% | 2% | 2.00% | 1.5% |

TABLE 22-3
Hydration and Skin Barrier Repair

| | | Methylation | Phenotype | Average (Methylation, | |
|---|---|---|---|---|---|
| Hydration and Skin | Genotype | Age | Age | Phenotype age) + | |
| Barrier Repair | At least 1 genetic issue | 31-40 | 51-60 | Genotype | Scaled |
| Peptide Name | | | | | |
| Decapeptide-4 | | 1% | | 1.00% | 0.8% |
| Acetyl Hexapeptide-37 (Diffuporine) | 0.50% | 0.50% | | 1.00% | 0.8% |
| Oligopeptide-24 (EGF-like peptide) | | | | | |
| FSS Ceramide 3 | | 1% | 1% | 1.00% | 0.8% |
| SODIUM pca | | | | | |
| APT-GPF | 0.50% | 1% | 1% | 1.50% | 1.1% |

TABLE 22-4
Anti-inflammatory and Soothing

| | Genotype | Methylation | Phenotype | Average(Methylation, | |
|---|---|---|---|---|---|
| | At least 1 | Age | Age | Phenotype age) + | |
| | genetic issue | 51-60 | 41-50 | Genotype | Scaled |
| Peptide Name | | | | | |
| Acetyl Hexapeptide-49 (Delisens) | 1% | 2% | 1% | 2.50% | 1.9% |
| Palmitoyl tetrapeptide-7 | 1% | 1% | 1% | 2.00% | 1.5% |
| Acetyl Tetrapeptide-15 (Skinasensyl) | 0.50% | | | | |
| Polyquaternium-51 | | 0.50% | | 0.50% | 0.4% |
| Allantoin | 0.10% | 0.10% | 0.10% | 0.20% | 0.2% |
| Hesperdine Methyl Chalcone | 0.30% | 0.40% | | 0.70% | 0.5% |
| Acetyl Tetrapeptide-40 (TELANGYN) | | | | | |
| Acetyl Dipeptide-3 Aminohexanoate | | | | | |
| (Bodyfensine) | | | | | |
| Oligopeptide-10 | | | | | |
| Acetyl Tetrapeptide-2 (Syn-UP) | 0.50% | 1% | | 1.50% | 1.1% |

TABLE 22-5
Brightening and Pigmentation

| | Genotype | Methylation | Phenotype | Average(Methylation, | |
|---|---|---|---|---|---|
| | At least 1 | Age | Age | Phenotype age) + | |
| | genetic issue | 41-50 | 51-60 | Genotype | Scaled |
| Peptide Name | | | | | |
| Hexapeptide-2 | | | | | |
| Nonapeptide-1 (Melanostatine) | 3% | 3% | 4% | 6.50% | 4.9% |
| Decapeptide 12 | 1.00% | 2% | 3% | 3.50% | 2.6% |
| Oligopeptide-34 | | | | | |
| Oligopeptide-51 | | | | | |

TABLE 22-6
Lymphatic drainage

| | Genotype | Methylation | Phenotype | Average(Methylation, | |
|---|---|---|---|---|---|
| | No Genetic | Age | Age | Phenotype age) + | |
| | issue | 31-40 | 31-40 | Genotype | Scaled |
| Peptide Name | | | | | |
| Eyesery1-Acetyl Tetrapeptide-5 | | | | | |
| Caffeine | | 0.20% | 0.20% | 0.20% | 0.2% |

TABLE 22-7
UV/Oxidative Damage/DNA/Cell damage

| | At least 1 genetic issue | 51-60 | 61-70 | | |
|---|---|---|---|---|---|
| Peptide Name | | | | | |
| DAP Tripeptide 33 | 0.50% | 2% | 3% | 3.00% | 2.3% |
| Pepha tight | 1% | 0.50% | 0.50% | 1.00% | 0.8% |
| Ferulic acid | 0.50% | | | | |
| Acetyl Tetrapeptide-22 (Thermostressine) | | | | | |
| Acetyl Heptapeptide-1 | | | | | |
| Tripeptide-9 Citrulline (dGlyage) | | | | | |

TABLE 22-8
Prebiotic and Probiotic

| | | | |
|---|---|---|---|
| Pepha active | 1% | 1.00% | 0.8% |
| Beta Glucan | 1% | 1.00% | 0.8% |
| Bacillus Extract | 1% | 1.00% | 0.8% |
| Sea kelp extract | 2% | 2.00% | 1.5% |
| Alpaflor Sebum | | | |

TABLE 22-9
Vitamins

| | | | | |
|---|---|---|---|---|
| D Panthenol | | 3% | 3.00% | 2.3% |
| Magnesium Ascorbyl Phosphate | 0.20% | 1% | 1.20% | 0.9% |
| Tocopherols | | 0.10% | 0.10% | 0.1% |
| Niacinamide | | 0.10% | 0.10% | 0.1% |

1. A method for determining a customized skin regimen for a subject, the method comprising:
for one or more skin activity categories, each skin activity category comprising a plurality of elements being considered for inclusion in the customized skin regimen:
obtaining or having obtained one or more of:
genetic statuses of one or more genomic locations related to the skin activity category from one or more samples obtained from the subject;
methylation statuses of one or more genes involved in biological processes of the skin activity category; and
an image captured of the subject's skin;
determining a methylation score, a phenotype score, and a genetic score for the skin activity category for the subject using the one or more of genetic statuses, methylation statuses, and the image;
for each of one or more of the methylation score, phenotype score, and genetic score, determining a fixed value for each of the plurality of elements of the skin activity category using the corresponding score; and
for each element of the plurality of elements, combining fixed values across the one or more of the methylation score, phenotype score, and genetic score to determine a percentage of the element to be included in the customized skin regimen.

2. The method of claim 1, wherein the one or more skin activity categories comprise an anti-aging and wrinkle reduction category, a lymphatic drainage category, a skin firming and elasticity category, a brightening and pigmentation category, a UV/oxidative damage/DNA/cell damage category, an anti-inflammatory and soothing category, and a hydration and skin barrier repair category.

3. The method of claim 1 or 2, wherein determining the methylation score for the skin activity category for the subject comprises combining methylation levels of one or more genes in the skin activity category.

4. The method of claim 3, wherein the skin activity category is the anti-aging and wrinkle reduction category, and wherein the one or more genes include one or more of CDKN2A, COL1A1, MMP1, and SIRT.

5. The method of claim 3 or 4, wherein the skin activity category is the lymphatic drainage category and wherein the one or more genes include one or more of NFAT5, VEGFC, and PROX1.

6. The method of any of claims 3-5, wherein the skin activity category is the skin firming and elasticity category and wherein the one or more genes include one or more of ELN, KLF4, and WRN.

7. The method of any of claims 3-6, wherein the skin activity category is the brightening and pigmentation category and wherein the one or more genes include one or more of IGF2 and TYR.

8. The method of any of claims 3-7, wherein the skin activity category is the UV/oxidative damage/DNA/cell damage category and wherein the one or more genes include one or more of TP53, BCL2, and SYN1.

9. The method of any of claims 3-8, wherein the skin activity category is the anti-inflammatory and soothing category and wherein the one or more genes include one or more of TNF-a, CSCL8, and IL-6.

10. The method of any of claims 3-9, wherein the skin activity category is the hydration and skin barrier repair category and wherein the one or more genes include one or more of FILAGGRIN (FLG), TGM1, CSCL8, CLDN1, and AQP3.

11. The method of any of claims 3-10, wherein combining methylation levels of one or more genes in the skin activity category is determined according to a formula: methylation age in each category=Σ(methylation level of gene×weight).

12. The method of claim 11, wherein each weight for the one or more genes in the skin category is between 0.1 and 0.3.

13. The method of any of claims 1-12, wherein determining the phenotype score for the skin activity category comprises determining one or more values by analyzing the image of the subject.

14. The method of claim 13, wherein determining one or more values comprises manually analyzing the image for characteristics relevant to the skin activity category.

15. The method of claim 13, wherein determining one or more values comprises applying an automated process to analyze the image for characteristics relevant to the skin activity category.

16. The method of any one of claims 13-15, wherein determining the phenotype score further comprises determining a second value based on answers included on a skin questionnaire including a set of questions related to the skin activity category.

17. The method of claim 16, wherein determining the phenotype score for the skin activity category comprises combining the one or more values determined by analyzing the image and the second value determined based on answers included on the skin questionnaire.

18. The method of claim 17, wherein determining the phenotype score further comprises mapping the combined one or more values and the second value to the phenotype score.

19. The method of any one of claims 1-18, wherein determining the genetic score comprises determining a quantity of genetic issues in the subject.

20. The method of claim 19, wherein the quantity of genetic issues comprises one or more genetic statuses indicative of a mutation.

21. The method of any one of claims 1-20, wherein determining a fixed value for each of the plurality of elements comprises accessing a look up table.

22. The method of any one of claims 1-21, wherein at least two of the plurality of elements have different determined fixed values.

23. The method of any one of claims 1-22, wherein combining fixed values across the one or more of the methylation score, phenotype score, and genetic score comprises determining an average of fixed values of the methylation score and the phenotype score.

24. The method of claim 23, wherein combining fixed values across the one or more of the methylation score, phenotype score, and genetic score further comprises summating the determined average of fixed values of the methylation score and the phenotype score and a fixed value of the genetic score.

25. The method of claim 24, wherein combining fixed values across the one or more of the methylation score, phenotype score, and genetic score further comprises scaling the summated value to determine the percentage of the element to be included in the customized skin regimen.

26. The method of any one of claims 1-25, wherein the plurality of elements for each skin activity category comprises one or more of peptides.

27. The method of claim 26, wherein the plurality of elements for the anti-aging and wrinkle reduction category comprise one or more of palmitoyl pentapeptide-3 (Matrixyl), acetyl dipeptide-1 cetyl ester (Idealift), acetyl hexapeptide-8 (Argireline), acetyl hexapeptide-30 (Inyline®-Peptide), acetyl hexapeptide-51 amide (Juveleven), Argirelox (Pentapeptide 18+Argireline), SNAP 8-acetyl octapeptide-3, Tripeptide 5, GHK Cu, Reproage™, Synake, or palmitoyl pentapeptide 38.

28. The method of claim 26 or 27, wherein the plurality of elements for the lymphatic drainage category comprise eyeseryl-acetyl tetrapeptide-5 or caffeine.

29. The method of any one of claims 26-28, wherein the plurality of elements for the skin firming and elasticity category comprise one or more of palmitoyl hexapeptide-12 (Biopeptide EL), palmitoyl oligopeptide (Bio-bustyl), myristoyl pentapeptide-8 (SymPeptide 225), palmitoyl oligopeptide (Biopeptide CL), acetyl heptapeptide 4 (Fensebiome), triptide-10 citrulline (Decorinyl), hexapeptide-10 (Serilesine), acetyl tetrapeptide-11 (Syniorage), acetyl hexapeptide-38 (Adifyline), hexapeptide-11 (Peptide Vinci 2), acetyl dipeptide-13 diphenylglycine (Relistase), palmitoyl dipeptide-5 diaminobutyloyl hydroxythreonine (and) palmitoyl dipeptide-6 diaminohydroxybutyrate (Syntacks), or acetyl tetrapeptide-2 (Uplevity).

30. The method of any one of claims 26-29, wherein the plurality of elements for the hydration and skin barrier repair category comprise one or more of Decapeptide-4, acetyl hexapeptide-37 (Diffuporine), Oligopeptide-24 (EGF-like peptide), FSS Ceramide 3, sodium pca, or APT-GPF.

31. The method of any one of claims 26-30, wherein the plurality of elements for the anti-inflammatory and soothing category comprise one or more of acetyl hexapeptide-49 (Delisens), palmitoyl tetrapeptide-7, acetyl tetrapeptide-15 (Skinasensyl), polyquaternium-51, allantoin, hesperdine methyl chalcone, acetyl tetrapeptide-40 (TELANGYN), acetyl dipeptide-3 aminohexanoate (Bodyfensine), Oligopeptide-10, or Acetyl Tetrapeptide-2 (Syn-UP).

32. The method of any one of claims 26-31, wherein the plurality of elements for the brightening and pigmentation category comprise one or more of Hexapeptide-2, Nonapeptide-1 (Melanostatine), Decapeptide 12, Oligopeptide-34, or Oligopeptide-51.

33. The method of any one of claims 26-32, wherein the plurality of elements for the UV/Oxidative Damage/DNA/Cell damage category comprise one or more of DAP tripeptide 33, pepha tight, ferulic acid, acetyl tetrapeptide-22 (Thermostressine), acetyl heptapeptide-1, or tripeptide-9 citrulline (dGlyage).

34. The method of any one of claims 1-33, wherein the plurality of elements further comprises one or more prebiotics or probiotics.

35. The method of claim 34, wherein the one or more prebiotics or probiotics comprise one or more of Pepha active, beta glucan, Bacillus extract, sea kelp extract, or alpaflor sebum.

36. The method of any one of claims 1-35, wherein the plurality of elements further comprises one or more vitamins.

37. The method of claim 36, wherein the one or more vitamins comprises one or more of D-Panthenol, magnesium ascorbyl phosphate, tocopherols, or niacinamide.

38. The method of any one of claims 1-37, wherein the plurality of elements further comprises one or more base elements.

39. The method of any one of claims 1-38, further comprising:
i. obtaining or having obtained levels of a plurality of skin biomarkers from one or more samples obtained from the subject;
ii. obtaining or having genetic statuses of one or more genomic locations of a plurality of genes from the one or more samples obtained from the subject; and
iii. determining the customized skin regimen for the subject using both the levels of the plurality of skin biomarkers and the genetic statuses of one or more genomic locations of a plurality of genes.

40. The method of claim 39, wherein the skin biomarkers comprise one or more collagens and elastins obtained from serum of the subject.

41. The method of claim 40, wherein the collagen biomarkers comprise one or more of amino-terminal propeptide of Collagen 1 (PINP), carboxy-terminal propeptide of type 1 collagen (PICP), crosslinked telopeptide of type 1 collagen (ICTP), amino-terminal propeptide of collagen 3 (PIIINP), matrix metalloproteinases-1 (MMP-1), tumor necrosis factor-alpha (TNF-α), insulin-like growth factor-1 (IGF-1), or basic fibroblast growth factor (FGF-2).

42. The method of claim 40 or 41, wherein the elastin biomarkers comprise one or more of matrix metalloproteinases-12 (MMP-12) or elastin-derived peptides (EDPs).

43. The method of any one of claims 40-42, wherein the serum is collected using one or more of ethylenediaminetetraacetic acid (EDTA), serum separator tube (SST), dried blood spot, or ficoll tube.

44. The method of any one of claims 40-43, wherein the levels of the plurality of skin biomarkers are obtained by using one or more of microarray or mass spectrometry.

45. The method of claim 44, wherein the skin biomarkers comprise one or more lipids obtained from sebum of the subject.

46. The method of claim 45, wherein the sebum is obtained using an absorbent medium or material.

47. The method of claim 45 or 46, wherein the one or more lipids comprise one or more of glycerol lipids, free fatty acids, or cholesterol.

48. The method of any one of claim 1-47, wherein the skin biomarkers from the one or more samples obtained from the subject comprise skin biomarkers from urine of the subject.

49. The method of claim 48, wherein the skin biomarkers from urine of the subject comprise one or more UV damage metabolites and oxidative stress loads from the urine of the subject.

50. The method of any one of claims 39-49, wherein the skin biomarkers comprise one or more advanced glycation end products from urine or blood of the subject.

51. The method of claim 50, wherein the advanced glycation end products comprise one or more of carboxymethyl lysine (CEL), carboxymethyl lysine (CML), glucosepane, or pentosidine.

52. The method of any one of claims 39-51, wherein the skin biomarkers comprise one or more microorganisms from skin niches of the subject.

53. The method of claim 52, wherein the microorganisms comprise one or more of Propionibacterium acnes, Propionibacterium avidum, or Propionibacterium granulosum, optionally from forehead of the subject.

54. The method of claim 52, wherein the microorganisms comprise Propionibacterium acnes, optionally from cheek of the subject.

55. The method of claim 52, wherein the microorganisms comprise one or more of Staphylococcus epidermidis, Corynebacterium spp, or Propionibacterium acnes, optionally from nasolabial fold of the subject.

56. The method of claim 52, wherein the microorganisms comprise Propionibacterium acnes, optionally from chin of the subject.

57. The method of claim 52, wherein the microorganisms comprise Firmicutes, optionally from face of the subject.

58. The method of claim 52, wherein the microorganisms comprise one or more of Corynebacterium kroppenstedtiin or Staphylococcus epidermidis, optionally from cheek of the subject.

59. The method of claim 52, wherein the microorganisms comprise Corynebacterium kroppenstedtiin, optionally from nasolabial fold of the subject.

60. The method of claim 52, wherein the microorganisms comprise Staphylococcus epidermidis, optionally from eyelid of the subject.

61. The method of claim 52, wherein the microorganisms comprise Staphylococcus epidermidis, optionally from cheek of the subject.

62. The method of claim 52, wherein the microorganisms comprise Corynebacterium species, optionally from chin of the subject.

63. The method of claim 52, wherein the microorganisms comprise Malassezia spp, optionally from cheek of the subject.

64. The method of claim 52, wherein the microorganisms comprise one or more Eikenella corrodens, Cutibacterium, Micrococcus luteus, or Kocuria, optionally from cheek of the subject.

65. The method of claim 52, wherein the microorganisms comprise Acinetobacter, optionally from cheek of the subject.

66. The method of claim 52, wherein the microorganisms comprise Corynebacterium, optionally from cheek of the subject.

67. The method of claim 52, wherein the microorganisms comprise one or more of Corynebacterium amycolatum, Proteobacteria, Firmicutes, Bacteroidetes, or Propionibacterium, optionally from forehead of the subject.

68. The method of claim 52, wherein the microorganisms comprise Propionibacterium, optionally from cheek of the subject.

69. The method of claim 52, wherein the microorganisms comprise one or more of Corynebacterium, Lactobacillus, or Cutibacterium, optionally from face of the subject.

70. The method of any one of claims 39-69, wherein the skin biomarkers comprise one or more hormones from urine or blood of the subject.

71. The method of claim 70, wherein, when the subject is a male, the hormones comprise one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG).

72. The method of claim 70, wherein, when the subject is a male, obtaining the levels of a plurality of skin biomarkers further comprises determining one or more combinations of levels of the one or more hormones.

73. The method of claim 72, wherein determining one or more combinations of levels of the one or more hormones comprises determining one or more of Estriol (E3)/(E1+E2), 2-OH (E1+E2)/16a-OH E1, 2-MeO E1/2-OH E1, 4-MeO E1/4-OH E1, 4-MeO E2/4-OH E2, testosterone (T)/Epi-T, b-pregnanediol/E2, cortisol/cortisone, or metabolized cortisol (THF+THE), or Prostaglandin (Pg)/E2.

74. The method of claim 70, wherein, when the subject is a female, the hormones comprise one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5a-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG).

75. The method of claim 70, wherein, when the subject is a female, obtaining the levels of a plurality of skin biomarkers further comprises determining one or more combinations of levels of the one or more hormones.

76. The method of claim 75, wherein determining one or more combinations of levels of the one or more hormones comprises determining one or more of E3/(E1+E2), 2-OH (E1+E2)/16a-OH E1, 2-MeO E1/2-OH E1, 4-MeO E1/4-OH E1, 4-MeO E2/4-OH E2, T/Epi-T, b-pregnanediol/E2, cortisol/cortisone, or metabolized cortisol (THF+THE), or Prostaglandin (Pg)/E2.

77. The method of any one of claims 39-76, wherein the skin biomarkers comprise one or more metabolism markers from serum or urine of the subject.

78. The method of claim 77, wherein the metabolism markers comprise one or more serum metabolism biomarkers.

79. The method of claim 78, wherein the serum metabolism biomarkers comprise one or more of lipid metabolism biomarkers, carbohydrate metabolism biomarkers, or cellular biomarkers.

80. The method of claim 79, wherein the lipid metabolism biomarkers comprise one or more of vaccenic acid, adiponectin, acylhexosylceramide (AHC), diacylglycerol (DAG), dimethyl-phosphatidylethanolamine (DMPE), or plasminogen activator inhibitor-1 (PAI-1).

81. The method of claim 79 or 80, wherein the carbohydrate metabolism biomarkers comprise one or more of adiponectin, gastric-inhibitor-peptide (GIP), or leptin.

82. The method of any one of claims 79-81, wherein the cellular biomarkers comprise nicotinamide adenine dinucleotide (NAD).

83. The method of claim 77, wherein the metabolism markers comprise one or more urine metabolism biomarkers.

84. The method of claim 83, wherein the urine metabolism biomarkers comprise one or more of lipid metabolism biomarkers or carbohydrate metabolism biomarkers.

85. The method of claim 84, wherein the lipid metabolism biomarkers comprise one or more of adipate, suberate, or ethyl malonate.

86. The method of claim 83 or 84, wherein the carbohydrate metabolism biomarkers comprise one or more of pyruvate, L-lactate, or β-hydroxybutyrate.

87. The method of any one of claims 39-86, wherein the skin biomarkers comprise one or more nutrients from the subject.

88. The method of claim 87, wherein the nutrients comprise one or more vitamins or minerals.

89. The method of claim 88, wherein the vitamins or minerals comprise one or more of Folate (L-5-methyltetrahydrofolate), magnesium, iron, vitamin A (retinol), vitamin D3 (cholecalciferol), vitamin E, choline, free Carnitine, vitamin B1 (thiamine diphosphate), vitamin B2 (riboflavin 5-Phosphate), vitamin B5 (pantothenic acid), vitamin B6 (pyridoxal 5-Phosphate), vitamin C (L-ascorbic acid), copper, zinc, selenium, chromium, or manganese.

90. The method of claim 87, wherein the nutrients comprise one or more vitamins and minerals, amino acids, or fatty acids.

91. The method of claim 90, wherein the vitamins and minerals comprise vitamin A (retinol), vitamin A (beta-carotene), vitamin B1 (thiamine diphosphate), vitamin B2 (riboflavin 5-phosphate), vitamin B3 (nicotinic acid), vitamin B5 (pantothenic acid), vitamin B6, pyridoxal 5-phosphate, vitamin B7 (biotin), vitamin B12 (cyanocobalamin), vitamin C (L-ascorbic acid), vitamin D (25-OH), vitamin D3 (cholecalciferol), vitamin D (1-25 dihydroxy), vitamin E (alpha-tocopherol), vitamin K1 (phylloquinone), vitamin K2 (menaquinone-MK-7), folate (L-5-methyltetrahydrofolate), coenzyme Q10 (ubiquinone+ubiquinol), selenium, sodium, potassium, calcium, zinc, manganese, iron, magnesium, copper, chromium, myoinositol, iodine, molybdenum, phosphorus, tetrahydrobiopterin, or fluoride, copper/zinc.

92. The method of claim 91, further comprising determining one or more combinations of levels of one or more of the vitamins and minerals.

93. The method of claim 92, wherein determining one or more combinations of levels of one or more of the vitamins and minerals further comprises obtaining a ratio of copper/zinc.

94. The method of claim 90, wherein the amino acids comprise one or more of glutathione (oxidized), methylmalonic acid (MMA), choline, L-cysteine, L-asparagine, L-glutamine, L-serine, L-arginine, L-citrulline, L-isoleucine, L-valine, L-leucine, free carnitine, or phenylalanine.

95. The method of claim 90, wherein the fatty acids comprise one or more of docosahexaenoic acid (DHA), Eicosapentaenoic acid (EPA), docosapentaenoic acid (DPA), arachidonic acid (AA), or linoleic acid (LA).

96. The method of claim 95, wherein obtaining the levels of a plurality of skin biomarkers further comprises determining one or more combinations of levels of one or more of the fatty acids.

97. The method of claim 96, wherein determining one or more combinations of levels of one or more of the fatty acids further comprises obtaining omega-3 total, omega-6 total, omega-3 index, or a ratio of AA/EPA.

98. The method of any one of claims 39-97, wherein the skin biomarkers comprise one or more commensal microorganisms from the subject.

99. The method of claim 98, wherein the commensal microorganisms comprise one or more of commensal bacteria, archaea, fungi, or viruses.

100. The method of claim 98 or 99, wherein the skin biomarkers further comprise gut metabolites from the subject.

101. The method of any one of claims 39-100, further comprising:
obtaining or having obtained one or more demographics of the subject; and
determining the customized skin regimen for the subject based on the demographics of the subject.

102. The method of claim 90, wherein the demographics comprise one or more of an age or gender of the subject.

103. The method of any one of claims 39-102, further comprising:
obtaining or having obtained an image of a face from the subject;
grading severities of one or more phenotypic parameters for the face of the subject based on the image; and
determining the customized skin regimen for the subject based on the severity of the one or more phenotypic parameters.

104. The method of claim 103, wherein the phenotypic parameters comprise one or more of wrinkles, periorbital-around eyes (puffy eyes), nasolabial folds (laugh lines), worry lines, uneven skin tone/pigmentation, or age spots (liver spots).

105. The method of any one of claims 39-104, further comprising:
obtaining or having obtained one or more responses to a skin questionnaire from the subject; and
determining the customized skin regimen for the subject based on the one or more responses from the subject.

106. The method of claim 105, wherein the skin questionnaire comprises one or more questions related to skin goals, type of skin, sun exposure, or skin proneness to external factors.

107. A method for providing a customized skin regimen to a subject, the method comprising:
administering the customized skin regimen to the subject, wherein the customized skin regimen is determined according to a method of any one of claims 1-106.

108. A customized skin regimen, wherein the customized skin regimen is determined according to a method of any one of claims 1-106.

109. The customized skin regimen of claim 108, wherein:
i. a skin biomarker is the Amino terminal propeptide of Collagen 1 (PINP), and the composition comprises one or more of vitamin C, peptides, retinoids, copper peptides, copper, collagen or collagen peptides, and/or omega-3 fatty acids;
ii. a skin biomarker is the Carboxy terminal propeptide of type 1 collagen (PICP) and the composition comprises one or more of hyaluronic acids, retinoids, vitamin C, niacinamide, collagen or collagen peptides, zinc, and/or omega-3 fatty acids;
iii. a skin biomarker is the Crosslinked telopeptide of type 1 collagen (ICTP), and the composition comprises one or more of retinoids, vitamin C, peptides, proline, glycine, vitamin E, vitamin C, and/or copper;
iv. a serological skin biomarker is the Amino terminal propeptide of Collagen 3 (PIIINP), and the composition comprises one or more of alpha hydroxy acids, retinoids, vitamin C, peptides, glycine, proline, and/or omega-3 fatty acids;
v. a skin biomarker is the Matrix metalloproteinase-1 (MMP-1) and composition comprises one or more of ginseng, green tea extract, retinoids, vitamin C, peptides, collagen or collagen peptides, vitamin E, vitamin C, and/or omega-3 fatty acids;
vi. a skin biomarker is Insulin like growth factor-1 (IGF-1) and the composition comprises one or more of capsaicin, vitamin C, hyaluronic acid, zinc, vitamin C, probiotics, collagen or collagen peptides, and/or omega-3 fatty acids;
vii. a skin biomarker is Basic fibroblast growth factor (FGF-2) and the composition comprises one or more of ginseng, vitamin C, hyaluronic acid, collagen or collagen peptides, resveratrol, glycine, and/or proline;
viii. a skin biomarker is Matrix Metalloproteinase-12 (MMP-12) and the composition comprises one or more of vitamin C, green tea extract, silicon, ginseng, and/or vitamin E;
ix. a skin biomarker is Elastin derived peptides (EDPs), and the composition comprises one or more of niacinamide, hyaluronic acid, silicon, vitamin A, and/or aloe vera.

110. The customized skin regimen of claim 108 or 109, wherein the peptide is one or more of Hexapeptide-11, GHK-Cu, Palmitoyl Pentapeptide-4 (Pal-KTTKS), Palmitoyl Tetrapeptide-7 (Pal-GQPR), Dipeptide-2, Palmitoyl Pentapeptide-3, decapeptide-12, Dipeptide Diaminobutyroyl Benzylamide Diacetate, Palmitoyl Dipeptide-5 Diaminobutyroyl Hydroxythreonine, Palmitoyl Dipeptide-5 Diaminohydroxybutyrate, Tetradecyl Aminobutyroylvalylaminobutyric Urea Trifluoroacetate, Acetyl Tetrapeptide-5, Heptapeptide-4, Acetyl Hexapeptide-51 Amide, Acetyl Hexapeptide-30, Acetyl Hexapeptide-8 (Argireline), acetyl hexapeptide-3 (Argireline), Acetylarginyltryptophyl Diphenylglycine, Acetyl Hexapeptide-37, Hexapeptide-10, Acetyl Octapeptide-3, Tetrapeptide-1, Tetrapeptide-2, Acetyl Tetrapeptide-2, Acetyl Hexapeptide-38, Acetyl Dipeptide-3 Aminohexanoate, Tripeptide-10 Citrulline (Decorinyl), Acetyl Hexapeptide-49, Pentapeptide 18, Tripeptide 29, Nonapeptide-1, Tripeptide 5, Palmitoyl Tripeptide-5, Diaminopropionoyl (DAP) Tripeptide-33, beta glucan, and/or bacillus extract.

111. The customized skin regimen of any one of claims 108-110, wherein the retinoid is one or more of retinol, retinal, tretinoin (retinoic acid), isotretinoin, alitretinoin, adapalene, bexarotene, tazarotene, and/or Trifarotene.

112. The customized skin regimen of any one of claims 108-111, wherein the omega-3 fatty acid is one or more of Alpha-linolenic acid (ALA), Eicosapentaenoic acid (EPA), and/or Docosahexaenoic acid (DHA).

113. The customized skin regimen of any one of claims 108-112, wherein the Alpha hydroxy acid is one or more of glycolic acid, lactic acid, mandelic acid, and/or citric acid.

114. The customized skin regimen of any one of claims 108-113, wherein:
x. a polymorphism is a SNP associated with melanin synthesis and the composition comprises one or more of Hydroquinone, Retinoids, Azelaic acid, Kojic acid, Cysteamine, Lignin peroxidase, Niacinamide, Ellagic acid, L-Cystine, L-Glutathione, Polypodium leucotomos, Melatonin, Arbutin, and/or Vitamin C;
xi. a polymorphism is a SNP associated with Moisturization and the composition comprises one or more of Vitamin E, Coenzyme Q10, Vitamin F, Kumazasa extract, Magnesium ascorbyl phosphate, Hyaluronic acid, Magnesium oil, collagen, ceramides, hyaluronan, procyanidin, probiotics, Galactooligosaccharides, Alpha-linolenic acid, and/or Gamma-linolenic acid;
xii. a polymorphism is a SNP associated with Skin Inflammation and the composition comprises one or more of Coenzyme Q10, Methylsulfonylmethane, Retinoid, Vitamin E, Vitamin C, Curcumin, Green tea or green tea extract, and/or Omega 3 fatty acids;
xiii. a polymorphism is a SNP associated with Melanocyte damage and the composition comprises one or more of Coenzyme Q10, Methylsulfonylmethane, Retinoid, Vitamin E, Vitamin D, Zinc, Vitamin C, Curcumin, Green tea or green tea extract, and/or Omega 3 fatty acids;
xiv. a polymorphism is a SNP associated with Collagen degradation and the composition comprises one or more of Retinoids, Vitamin C, Vitamin E, Tretinoin, Collagen peptides, Red ginseng, Aloe vera extract, Hyaluronic acid, Ginkgo biloba, and/or Soy;
xv. a polymorphism is a SNP associated with Elastin synthesis and the composition comprises one or more of Retinoid, Vitamin C, Collagen or collagen peptides, Hyaluronic acid, Dexpanthenol, Vitamin E, Vitamin A, and/or Zinc;
xvi. a polymorphism is a SNP associated with Antioxidants and the composition comprises one or more of Vitamin C, Coenzyme Q10, Polyphenols, Vitamin E, Curcumin, Vitamin C, Vitamin E, Beta carotene, Flavanols, zinc, Lutein, Lycopene, Silymarin, and/or Green tea or green tea extract;
xvii. a polymorphism is a SNP associated with Vitamin A deficiency and the composition comprises one or more of Retinol, Vitamin E, Resveratrol, Baicalin, B-vitamins, Vitamin C, Epigallocatechin gallate, Collagen, Nicotinamide riboside, Nicotinamide mononucleotide, Coenzyme Q10, and/or Curcumin;
xviii. a polymorphism is a SNP associated with Vitamin B6 deficiency and the composition comprises one or more of B-vitamins, Vitamin B6, Coenzyme Q10, Methylsulfonylmethane; Retinoids, and Vitamin E, Curcumin, Green tea or green tea extract, Omega 3 fatty acids, and/or Vitamin C;
xix. a polymorphism is a SNP associated with Vitamin B12 deficiency and the composition comprises one or more of Retinol, Vitamin E, Resveratrol, Baicalin, B-vitamins, Vitamin B12, Vitamin C, Curcumin, Epigallocatechin gallate, Collagen, Nicotinamide riboside, Nicotinamide mononucleotide, and/or Coenzyme Q10;
xx. a polymorphism is a SNP associated with Vitamin C deficiency and the composition comprises one or more of Vitamin C, Coenzyme Q10, Polyphenols, Vitamin E, Curcumin, Beta carotene, Flavanols, zinc, Lutein, Lycopene, Silymarin, and/or Green tea or green tea extract;
xxi. a polymorphism is a SNP associated with Vitamin D deficiency and the composition comprises one or more of Vitamin D, Retinol, Vitamin E, Resveratrol, Baicalin, B-vitamins, Vitamin C, Curcumin, Epigallocatechin gallate, Collagen, Nicotinamide riboside, Nicotinamide mononucleotide, and/or Coenzyme Q10;
xxii. a polymorphism is a SNP associated with Folate deficiency and the composition comprises one or more of Folate, Retinol, Vitamin E, Resveratrol, Baicalin, B-vitamins, Vitamin C, Curcumin, Epigallocatechin gallate, Collagen, Nicotinamide riboside, Nicotinamide mononucleotide, and Coenzyme Q10; and/or
xxiii. a polymorphism is a SNP associated with Omega fatty acid deficiency and the composition comprises one or more of DHA (Docosahexaenoic acid), EPA, Coenzyme AL, Methylsulfonylmethane, Retinoid, Curcumin, Green tea or green tea extract and/or Vitamin E.

115. The customized skin regimen of any one of claims 108-114, wherein a phenotypic parameter is wrinkles, Periorbital-around eyes (puffy eyes), Nasolabial folds (Laugh lines), worry lines, Uneven Skin tone/Pigmentation, and/or Age spots (Liver spots) and the composition comprises one or more of Tretinoin, Zeaxanthin, Retinol, Retinoids, Vitamin C, Hyaluronic acid, Caffeine, Phytonadione, Vitamin E, Astaxanthin, Hyaluronic acid, Peptides, Niacinamide, Azelaic acid, Soybean extract, Kojic acid, Aloesin, and/or Turmeric.

116. The customized skin regimen of claim 105, further comprising one or more of Zeaxanthin, Collagen, Vitamin C, Resveratrol, Vitamin E, Vitamin C, Hyaluronic acid, Glutathione, Niacinamide, and/or Coenzyme Q10.

117. The customized skin regimen of any one of claims 108-116, wherein a skin questionnaire question is:
xxiv. UV and the composition comprises one or more of Zinc oxide, titanium oxide, Polypodium leucotomos, Vitamin E, Vitamin C, and/or Niacinamide;
xxv. Anti-wrinkling and the composition comprises one or more of Tretinoin, Zeaxanthin, Retinol, Vitamin C, Hyaluronic acid, Collagen, Resveratrol, and/or Vitamin E;
xxvi. Anti-pigmentation and the composition comprises one or more of Niacinamide, Azelaic acid, Vitamin C, Soybean extract, Retinoids, Kojic acid, Vitamin E, Aloesin, Turmeric, and/or Glutathione
xxvii. Moisturization and the composition comprises one or more of Hyaluronic acid, Glycerin, Shea butter, Jojoba oil, Hyaluronan, Ceramides, and Collagen;
xxviii. Radiant complexion and the composition comprises one or more of Vitamin C and/or Niacinamide,
xxix. Dry skin and the composition comprises Glycerin;
xxx. Combinational skin and the composition comprises Hyaluronic acid;
xxxi. Acne-prone skin and the composition comprises Benzoyl peroxide;
xxxii. Sun exposure and the composition comprises Zinc oxide and/or Titanium dioxide;
xxxiii. Sensitive skin—stinging or burning and the composition comprises Niacinamide
xxxiv. Sun and Humidity and the composition comprises one or more of Vitamin E, Zinc oxide, Astaxanthin, and/or Omega-3 fatty acids
xxxv. Wind and cold and the composition comprises one or more of Petroleum jelly, Omega-3 fatty acids, and/or Probiotics.

118. The customized skin regimen of any one of claims 108-113, wherein a hormone is:
xxxvi. one or more of estrone (E1), estradiol (E2), dehydroepiandrosterone (DHEA), testosterone, epi-testosterone (Epi-T), androstenedione (AD), androsterone, etiocholanolone, 5-alpha-dihydrotestosterone (5α-DHT), dehydroepiandrosterone sulfate (DHEA-S), progesterone (P), cortisol, cortisone, b-tetrahydrocortisol (b-THF), a-Tetrahydrocortisol (a-THF), b-Tetrahydrocortisone (b-THE), deoxycorticosterone, corticosterone, melatonin, or 8-hydroxy-2′-deoxyguanosine (8-OHdG); or
xxxvii. one or more of estrone (E1), estradiol (E2), Estriol (E3), total estrogen, 2-OH estradiol, 2-OH estrone, 4-OH estradiol, 4-OH estrone, dehydroepiandrosterone (DHEA), androstenedione, androsterone, etiocholanolone, 5-alpha-dihydrotestosterone (5α-DHT), 5α,3α-Androstanediol, 5β-Androstanediol, dehydroepiandrosterone sulfate (DHEA-S), progesterone, allopregnanolone, allopregnanediol, 3α-dihydroprogesterone, 20α-dihydroprogesterone, b-pregnanediol, a-pregnanediol, cortisol, cortisone, b-tetrahydrocortisol (b-THF), a-tetrahydrocortisol (a-THF), deoxycorticosterone, corticosterone, melatonin, or 8-hydroxy-2′-deoxyguanosine (8-OHdG); and
xxxviii. the composition comprises one or more of Calcium D-glucarate, Green Tea, Maca DIM (3,3′-diindolylmethane), Black Cohosh, Ginger, Cranberry, Milk Thistle, Hops, Red clover, Damiana, Chaste Tree Berry Extract, Vitamin B2, Fenugreek seed, Ashwagandha, Vitamin D3, Zinc, Tribulus, Shatavari, Zinc, Shatavari, DHA, EPA, pine nuts, Licorice Root Extract, Oregano, Curcumin, Sodium, Rhodiola rosea, Vitamin B6, Vitamin B12, Magnesium, Folate, Iodine, Selenium, Potassium, Magnesium, Melatonin, Huang-qin, St. John's-wort, SAMe (S-adenosyl methionine), and/or Trimethylglycine (betaine).

119. The customized skin regimen of any one of claims 108-118, wherein a metabolic biomarker is one or more of Vaccenic acid, Adiponectin, Acylhexosylceramide (AHC), Dimethyl-phosphatidylethanolamine (DMPE), PAI-1, GIP, Leptin, NAD, Adipate, Suberate, Ethyl malonate, Pyruvate, L-lactate, and/or β-hydroxybutyrate and the supplemental or topical is one or more of Conjugated Linoleic Acid, DHA, EPA, Vitamin D, Curcumin, Berberine Extract, Resveratrol, Vitamin B3, Vitamin B6, Vitamin E, Choline, Vitamin C, Tryptophan, Nicotinamide riboside, Resveratrol, Quercetin, Vitamin B2, L-carnitine, Vitamin B1, Vitamin B12, CoQ10 (Coenzyme Q10), Alpha lipoic acid, and/or Chromium.

120. The customized skin regimen of any one of claims 108-119, further comprising an excipient or carrier.

121. The customized skin regimen of claim 120, wherein the excipient or carrier is one or more of water, Caprylyl Glycol, butylene glycol, ultra low molecular weight (ulmw) hyaluronic acid, middle molecular weight (MMW) hyaluronic acid, and/or dimethyl isosorbide

122. A method for determining a customized skin regimen for a subject, the method comprising:
obtaining or having obtained levels of a plurality of skin biomarkers from one or more samples obtained from the subject;
obtaining or having genetic statuses of one or more genomic locations of a plurality of genes from the one or more samples obtained from the subject;
obtaining or having obtained a methylation score, phenotype score and a questionnaire score for the subject; and
determining the customized skin regimen for the subject using both the levels of the plurality of skin biomarkers and the genetic statuses of one or more genomic locations of a plurality of genes or using the methylation score, the phenotype score and the questionnaire score for the subject.
